(19) United States
(12) Patent Application Publication
(10) Pub. No.: US 2024/0254192 A1
(43) Pub. Date: Aug. 1, 2024

(54) CHIMERIC POLYPEPTIDES AND METHODS OF USE

(71) Applicant: BOARD OF REGENTS, THE UNIVERSITY OF TEXAS SYSTEM, Austin, TX (US)

(72) Inventors: Sattva S. NEELAPU, Houston, TX (US); Jingwei LIU, Houston, TX (US); Sridevi PATCHVA, Houston, TX (US); Yongfu TANG, Houston, TX (US)

(73) Assignee: BOARD OF REGENTS, THE UNIVERSITY OF TEXAS SYSTEM, Austin, TX (US)

(21) Appl. No.: 18/560,746

(22) PCT Filed: May 13, 2022
(86) PCT No.: PCT/US2022/029232

§ 371 Date: Nov. 14, 2023

Related U.S. Application Data
(60) Provisional application No. 63/188,936, filed on May 14, 2021.
(60) Provisional application No. 63/274,765, filed on Nov. 2, 2021.

(51) Int. Cl. C07K 14/705; C12N 5/074; C12N 5/0783

(52) U.S. Cl. CPC C07K 14/70532; C07K 14/70517; C07K 14/70578; C12N 5/0636; C12N 5/0646; C12N 5/0696; C07K 2319/02; C07K 2319/03; C12N 2510/00

(57) ABSTRACT

Aspects of the present disclosure provide compositions and methods for detecting, isolating, depleting, and/or purifying cells, including polypeptides, including chimeric polypeptides, useful in such methods. Various chimeric polypeptides are disclosed, along with use of such polypeptides as selection markers, transduction markers, and/or safety switches. Also disclosed are cells, including therapeutic cells such as T cells, NK cells, NKT cells, and iPSCs, comprising polynucleotides encoding one or more chimeric polypeptides.

This application claims priority to U.S. Provisional Patent Application Ser. No. 63/188,936, filed May 14, 2021, and also to U.S. Provisional Patent Application Ser. No. 63/274,765, filed Nov. 2, 2021, both of which applications are incorporated by reference herein in their entirety.

### BACKGROUND

### I. Technical Field

Aspects of the disclosure relate to the field of molecular biology. In particular, embodiments of the invention relate to chimeric polypeptides, engineered cells, and methods of use thereof.

### II. Background

Therapeutic cells targeting tumor antigens, such as CAR-NK cells, CAR T cells, and TCR-transduced T cells, are promising approaches for the treatment of various malignancies. Despite the successes of these therapies to date, several limitations remain, including disease relapse, high manufacturing cost, and toxicities. There is a need for compositions and methods for targeting therapeutic cells, such as for isolation, identification, and purification during manufacturing as well as and for use in selectively targeting such cells for elimination in the event of adverse events such as cytokine release syndrome and graft-versus-host disease.

### SUMMARY

Disclosed herein, in some aspects, are methods and compositions useful in detecting, isolating, depleting, and/or purifying cells. Accordingly, disclosed in some embodiments are chimeric polypeptides comprising one or more extracellular regions (e.g., from BCMA, Trop2, CD30, EGFR, or Her2) and a transmembrane domain. In some embodiments, the chimeric polypeptides further comprise one or more additional regions, e.g., a signal peptide, a hinge region, or an intracellular region. Also disclosed are engineered cells expressing such polypeptides and methods for detecting, isolating, depleting, and/or purifying such cells.

Embodiments of the present disclosure include nucleic acids, polynucleotides, polypeptides, proteins, peptides, constructs, vectors, cells, therapeutic cells, immune cells, engineered cells, methods for generating engineered cells, methods for detecting engineered cells, methods for isolating engineered cells, methods for depleting engineered cells, and methods for purifying engineered cells. Nucleic acids of the disclosure may encode one or more polypeptides of the disclosure, including one or more chimeric polypeptides. In some embodiments, a nucleic acid molecule of the disclosure encodes a chimeric polypeptide. In some embodiments, a nucleic acid molecule of the disclosure encodes two or more chimeric polypeptides. A chimeric polypeptide of the disclosure can include at least 1, 2, 3, or more of the following regions or domains: a signal peptide, an extracellular domain, a hinge region, a transmembrane domain, and an intracellular region. An engineered cell of the disclosure can comprise 1, 2, 3, 4, or more polynucleotides and/or polypeptides of the disclosure. Methods of the present disclosure can include at least 1, 2, 3, 4, or more of the following steps: introducing a polynucleotide into a cell, introducing a vector into a cell, introducing a polypeptide into a cell, expressing a polypeptide in a cell, expanding a population of cells, contacting a cell with an antigen-binding protein, contacting a cell with an antibody drug conjugate, and detecting a cell with an imaging agent.

Disclosed herein, in some embodiments, is a chimeric polypeptide comprising (a) an extracellular domain from B-cell maturation antigen (BCMA); (b) a hinge region from programmed cell death 1 ligand 1 (PDL1); (c) a transmembrane domain; and (d) an intracellular region. In some embodiments, the extracellular domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO:19. In some embodiments, the extracellular domain comprises SEQ ID NO:19. In some embodiments, the extracellular domain consists of SEQ ID NO:19. In some embodiments, the hinge region comprises SEQ ID NO:23. In some embodiments, the hinge region consists of SEQ ID NO:23. In some embodiments, the transmembrane domain is an alpha or beta chain of the T cell receptor or a transmembrane domain from CD28, CD3e, CD45, CD4, CD5, CD8, CD9, CD16, CD22, CD33, CD37, CD64, CD80, CD86, CD123, CD134, CD137 or CD154. In some embodiments, the transmembrane domain is a transmembrane domain from CD8a. In some embodiments, the transmembrane domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO:26. In some embodiments, the transmembrane domain comprises SEQ ID NO:26. In some embodiments, the transmembrane domain consists of SEQ ID NO:26. In some embodiments, the transmembrane domain is a transmembrane domain from PDL1. In some embodiments, the transmembrane domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO:25. In some embodiments, the transmembrane domain comprises SEQ ID NO:25. In some embodiments, the transmembrane domain consists of SEQ ID NO:25. In some embodiments, the intracellular region comprises the sequence RLR (SEQ ID NO:29). In some embodiments, the intracellular region consists of the sequence RLR (SEQ ID NO:29). In some embodiments, the intracellular region comprises SEQ ID NO:31. In some embodiments, the intracellular region consists of SEQ ID NO:31. In some embodiments, the intracellular region comprises SEQ ID NO:32. In some embodiments, the intracellular region consists of SEQ ID NO:32. In some embodiments, the intracellular region comprises an amino acid sequence at least 95% identical to SEQ ID NO:40. In some embodiments, the intracellular region comprises SEQ ID NO:40. In some embodiments, the intracellular region consists of SEQ ID NO:40. In some embodiments, the intracellular region comprises at most 25, 24, 23, 22, 21, 20, 19, 18, 17, 16, 15, 14, 13, 12, 11, 10, 9, 8, 7, 6, 5, 4, or 3 amino acids. In some embodiments, the chimeric polypeptide is less than or equal to 100 amino acids in length. In some embodiments, wherein the chimeric polypeptide does not comprise a signaling domain. In some embodiments, the chimeric polypeptide does not comprise an intracellular region from BCMA.

In some embodiments, the chimeric polypeptide comprises an amino acid sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO:1. In some embodiments, the chimeric polypeptide comprises SEQ ID NO:1. In some embodiments, the chimeric polypeptide consists of SEQ ID NO:1. In some embodiments, the chimeric polypeptide comprises an amino acid sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO:38. In some embodiments, the chimeric polypeptide comprises SEQ ID NO:38. In some embodiments, the chimeric polypeptide consists of SEQ ID NO:38. Also disclosed is a nucleic acid molecule comprising a nucleotide sequence encoding the chimeric polypeptide. In some embodiments, the nucleic acid molecule comprises a nucleic acid sequence at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO:2. In some embodiments, the nucleic acid molecule comprises SEQ ID NO:2. In some embodiments, the nucleic acid molecule comprises a nucleic acid sequence at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO:39. In some embodiments, the nucleic acid molecule comprises SEQ ID NO:2. Also disclosed is a vector comprising the nucleic acid molecule.

Also disclosed herein, in some embodiments, is a chimeric polypeptide comprising (a) a tissue-type plasminogen activator (tPA) signal peptide; (b) an extracellular domain from BCMA; (c) a hinge region; (d) a transmembrane domain; and (e) an intracellular region. In some embodiments, the tPA signal peptide comprises a sequence at least 80%, 81%, 82%, 83%, 84%, 85%, 86%, 87%, 88%, 89%, 90%, 91%, 92%, 93%, 94%, 95%, 96%, 97%, 98%, or 99% sequence identical to SEQ ID NO:34. In some embodiments, the tPA signal peptide comprises SEQ ID NO:34. In some embodiments, the tPA signal peptide consists of SEQ ID NO:34. In some embodiments, the extracellular domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO:19. In some embodiments, the extracellular domain comprises SEQ ID NO:19. In some embodiments, the extracellular domain consists of SEQ ID NO:19. In some embodiments, the hinge region comprises a CD8a hinge, a PDL1 hinge, an IgG4 hinge, an IgG1 hinge, or a CD34 hinge. In some embodiments, the hinge region is a hinge region from PDL1. the hinge region comprises SEQ ID NO:23. In some embodiments, the hinge region consists of SEQ ID NO:23. In some embodiments, the hinge region is a hinge region from CD8a. In some embodiments, the hinge region comprises SEQ ID NO:24. In some embodiments, the hinge region consists of SEQ ID NO:24. In some embodiments, the transmembrane domain is an alpha or beta chain of the T cell receptor or a transmembrane domain from CD28, CD3e, CD45, CD4, CD5, CD8, CD9, CD16, CD22, CD33, CD37, CD64, CD80, CD86, CD123, CD134, CD137 or CD154. In some embodiments, the transmembrane domain is a transmembrane domain from CD8a. In some embodiments, the transmembrane domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO:26. In some embodiments, the transmembrane domain comprises SEQ ID NO:26. In some embodiments, the transmembrane domain consists of SEQ ID NO:26. In some embodiments, the intracellular region is a portion of an intracellular region from CD8a. In some embodiments, the intracellular region comprises at most 10, 9, 8, 7, or 6 amino acids. In some embodiments, the intracellular region comprises SEQ ID NO:30. In some embodiments, the intracellular region consists of SEQ ID NO:30. In some embodiments, the chimeric polypeptide is less than or equal to 150 amino acids in length. In some embodiments, the chimeric polypeptide does not comprise a signaling domain. In some embodiments, the chimeric polypeptide does not comprise an intracellular region from BCMA. In some embodiments, the chimeric polypeptide comprises an amino acid sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO:3. In some embodiments, the chimeric polypeptide comprises SEQ ID NO:3. In some embodiments, the chimeric polypeptide consists of SEQ ID NO:3. Also disclosed is a nucleic acid molecule comprising a nucleotide sequence encoding the chimeric polypeptide. In some embodiments, the nucleic acid molecule comprises a nucleic acid sequence at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO:4. In some embodiments, the nucleic acid molecule comprises SEQ ID NO:4. Also disclosed is a vector comprising the nucleic acid molecule.

Further disclosed herein, in some embodiments, is a chimeric polypeptide comprising (a) an extracellular domain from CD30; (b) a transmembrane domain from CD30; and (c) an intracellular region from BCMA. In some embodiments, the extracellular domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO:20. In some embodiments, the extracellular domain comprises SEQ ID NO:20. In some embodiments, the extracellular domain consists of SEQ ID NO:20. In some embodiments, the transmembrane domain comprises an amino acid sequence at least 95% identical to SEQ ID NO:27. In some embodiments, the transmembrane domain comprises SEQ ID NO:27. In some embodiments, the transmembrane domain consists of SEQ ID NO:27. In some embodiments, the intracellular region comprises an amino acid sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO:33. In some embodiments, the intracellular region comprises SEQ ID NO:33. In some embodiments, the intracellular region consists of SEQ ID NO:33. In some embodiments, the chimeric polypeptide does not comprise a signaling domain. In some embodiments, the chimeric polypeptide does not comprise an intracellular region from CD30. In some embodiments, the chimeric polypeptide comprises an amino acid sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO:9. In some embodiments, the chimeric polypeptide comprises SEQ ID NO:9. Also disclosed is a nucleic acid molecule comprising a nucleotide sequence encoding the chimeric polypeptide. In some embodiments, the nucleic acid molecule comprises a nucleic acid sequence at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO:10. In some embodiments, the nucleic acid molecule comprises SEQ ID NO:10. Also disclosed is a vector comprising the nucleic acid molecule.

Also disclosed herein, in some embodiments, is a chimeric polypeptide comprising (a) a signal peptide that is not a Her2 signal peptide; (b) an extracellular domain from Her2; and (c) a transmembrane domain from Her2. In some embodiments, the chimeric polypeptide does not comprise an intracellular region. In some embodiments, the signal peptide is a signal peptide from CD8a. In some embodiments, the signal peptide comprises an amino acid sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO:35. In some embodiments, the signal peptide comprises SEQ ID NO:35. In some embodiments, the signal peptide consists of SEQ ID NO:35. In some embodiments, the extracellular domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO:21. In some embodiments, the extracellular domain comprises SEQ ID NO:21. In some embodiments, the extracellular domain consists of SEQ ID NO:21. In some embodiments, the transmembrane domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO:28. In some embodiments, the transmembrane domain comprises SEQ ID NO:28. In some embodiments, the transmembrane domain consists of SEQ ID NO:28. In some embodiments, the chimeric polypeptide comprises an amino acid sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO:11. In some embodiments, the chimeric polypeptide comprises SEQ ID NO:11. In some embodiments, the chimeric polypeptide consists of SEQ ID NO:11. Also disclosed is a nucleic acid molecule comprising a nucleotide sequence encoding the chimeric polypeptide. In some embodiments, the nucleic acid molecule comprises a nucleic acid sequence at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO:12. In some embodiments, the nucleic acid molecule comprises SEQ ID NO:12. Also disclosed is a vector comprising the nucleic acid molecule.

Further disclosed herein, in some embodiments, is an engineered immune cell comprising a nucleic acid encoding a Trop2 polypeptide. In some embodiments, the Trop2 polypeptide comprises SEQ ID NO:15. In some embodiments, the nucleic acid comprises a nucleic acid sequence at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO:16. In some embodiments, the nucleic acid comprises SEQ ID NO:16.

Also disclosed herein, in some embodiments, is a chimeric polypeptide comprising: (a) a signal peptide; (b) an extracellular region comprising (i) EGFR domain III and (ii) a portion of EGFR domain IV having a length of less than 100 amino acids; (c) a hinge region; and (d) a transmembrane domain. In some embodiments, the portion of EGFR domain IV has a length of less than 75, 70, 65, 60, 55, 50, 45, 40, or 35 amino acids. In some embodiments, the portion of EGFR domain IV has a length of 33 amino acids. In some embodiments, the signal peptide is a signal peptide from GM-CSFRα. In some embodiments, the signal peptide comprises an amino acid sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO:36. In some embodiments, the signal peptide comprises SEQ ID NO:36. In some embodiments, the signal peptide consists of SEQ ID NO:36. In some embodiments, the extracellular region comprises an amino acid sequence at least 95% identical to SEQ ID NO:22. In some embodiments, the extracellular region comprises SEQ ID NO:22. In some embodiments, the extracellular region consists of SEQ ID NO:22. In some embodiments, the hinge region is a hinge region from CD8. In some embodiments, the hinge region comprises an amino acid sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO:37. In some embodiments, the hinge region comprises SEQ ID NO:37. In some embodiments, the hinge region consists of SEQ ID NO:37. In some embodiments, the transmembrane domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO:26. In some embodiments, the transmembrane domain comprises SEQ ID NO:26. In some embodiments, the transmembrane domain consists of SEQ ID NO:26. In some embodiments, the chimeric polypeptide does not comprise a signaling domain. In some embodiments, the chimeric polypeptide does not comprise an intracellular region from EGFR. In some embodiments, the chimeric polypeptide comprises an amino acid sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO:15. In some embodiments, the chimeric polypeptide comprises SEQ ID NO:15. In some embodiments, the chimeric polypeptide consists of SEQ ID NO:15. Also disclosed is a nucleic acid molecule comprising a nucleotide sequence encoding the chimeric polypeptide. In some embodiments, the nucleic acid molecule comprises a nucleic acid sequence at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO:16. In some embodiments, the nucleic acid molecule comprises SEQ ID NO:16. Also disclosed is a vector comprising the nucleic acid molecule.

In some embodiments, disclosed is a method of generating an engineered cell comprising introducing into a cell a chimeric polypeptide, a nucleic acid molecule, or a vector of the present disclosure.

In some embodiments, disclosed is an engineered cell comprising a nucleic acid molecule encoding for a chimeric polypeptide of the present disclosure. In some embodiments, the engineered cell is a T cell. In some embodiments, the T cell is a CD4⁺ T cell, CD8⁺ T cell, iNKT cell, NKT cell, γδ T cell, or regulatory T cell. In some embodiments, the engineered cell is a natural killer (NK) cell. In some embodiments, the engineered cell is an induced pluripotent stem cell (iPSC). In some embodiments, the engineered cell is an iPSC-derived cell. In some embodiments, the engineered cell further comprises a chimeric antigen receptor (CAR). In some embodiments, the chimeric polypeptide is operatively linked to the CAR. In some embodiments, the engineered cell further comprises a T cell receptor (TCR). In some embodiments, the chimeric polypeptide is operatively linked to the TCR. Also disclosed is a population of cells comprising an engineered cell of the disclosure.

Further disclosed is a method for detecting, isolating, depleting, or purifying an engineered cell of the disclosure, the method comprising contacting the engineered cell with an antigen-binding protein, where the antigen binding protein is capable of binding to a polypeptide of the engineered cell. In embodiments where the engineered cell expresses a chimeric polypeptide comprising an extracellular domain from BCMA, the antigen binding protein is a BCMA-binding protein. In embodiments where the engineered cell expresses a chimeric polypeptide comprising an extracellular domain from CD30, the antigen binding protein is a CD30-binding protein. In embodiments where the engineered cell expresses a chimeric polypeptide comprising an extracellular domain from Her2, the antigen binding protein is a Her2-binding protein. In embodiments where the engineered cell expresses a Trop2 polypeptide, the antigen binding protein is a Trop2-binding protein. In embodiments where the engineered cell expresses a chimeric polypeptide comprising an extracellular region comprising a portion of an extracellular domain from EGFR, the antigen binding protein is an EGFR-binding protein. In some embodiments, the antigen-binding protein is an antigen-specific antibody or antigen-binding fragment thereof. In some embodiments, the antigen-binding protein is linked to an imaging agent and the method further comprises detecting the cell with the imaging agent. In some embodiments, the antigen-binding protein is linked to a cytotoxic agent (e.g., is an antibody-drug conjugate). In some embodiments, contacting the engineered cell with the antigen-binding protein is performed in vitro. In some embodiments, contacting the engineered cell with the antigen-binding protein is performed ex vivo. In some embodiments, contacting the engineered cell with the antigen-binding protein is performed in vivo.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

The term “engineered” as used herein refers to an entity that is generated by the hand of man, including a cell, nucleic acid, polypeptide, vector, and so forth. In at least some cases, an engineered entity is synthetic and comprises elements that are not naturally present or configured in the manner in which it is utilized in the disclosure. In specific embodiments, a vector is engineered through recombinant nucleic acid technologies, and a cell is engineered through transfection or transduction of an engineered vector.

As used herein, “prevent,” and similar words such as “prevented,” “preventing” etc., indicate an approach for preventing, inhibiting, or reducing the likelihood of the occurrence or recurrence of, a disease or condition, e.g., cancer. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition.

The term “subject,” as used herein, generally refers to an individual having a biological sample that is undergoing processing or analysis and, in specific cases, has or is suspected of having cancer. The subject can be any organism or animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals. The subject can be a patient, e.g., have or be suspected of having a disease (that may be referred to as a medical condition), such as benign or malignant neoplasias, or cancer. The subject may being undergoing or having undergone treatment. The subject may be asymptomatic. The subject may be healthy individuals but that are desirous of prevention of cancer. The term “individual” may be used interchangeably, in at least some cases. The “subject” or “individual”, as used herein, may or may not be housed in a medical facility and may be treated as an outpatient of a medical facility. The individual may be receiving one or more medical compositions via the internet. An individual may comprise any age of a human or non-human animal and therefore includes both adult and juveniles (i.e., children) and infants and includes in utero individuals. It is not intended that the term connote a need for medical treatment, therefore, an individual may voluntarily or involuntarily be part of experimentation whether clinical or in support of basic science studies.

As used herein “treatment” or “treating,” includes any beneficial or desirable effect on the symptoms or pathology of a disease or pathological condition, and may include even minimal reductions in one or more measurable markers of the disease or condition being treated, e.g., cancer. Treatment can involve optionally either the reduction or amelioration of symptoms of the disease or condition, or the delaying of the progression of the disease or condition. “Treatment” does not necessarily indicate complete eradication or cure of the disease or condition, or associated symptoms thereof.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Any embodiment discussed with respect to one aspect of the disclosure applies to other aspects of the disclosure as well and vice versa. For example, any step in a method described herein can apply to any other method. Moreover, any method described herein may have an exclusion of any step or combination of steps. Aspects of an embodiment set forth in the Examples are also embodiments that may be implemented in the context of embodiments discussed elsewhere in a different Example or elsewhere in the application, such as in the Summary, Detailed Description, Claims, and Brief Description of the Drawings.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The embodiments of the disclosure may be better understood by reference to one or more of these drawings in combination with the detailed description of specific embodiments presented herein.

FIG. 1 shows GFP expression and BCMA staining of 293T cells transfected with plasmids containing one of three different membrane bound receptors: BCMA extracellular domain fused to PD-L1 hinge and transmembrane domain, BCMA extracellular domain fused to CD8a hinge and transmembrane domain, or full-length wild type BCMA.

FIG. 2 shows CD19 CAR and BCMA staining of primary T cells (transduced with BCL6 and BCL2L1) transduced with a lentiviral vector coexpressing a CD19 CAR and one of two different BCMA fusion constructs: BCMA extracellular domain with PD-L1 hinge and transmembrane domain but no functional intracellular domain (left panel) or BCMA extracellular domain fused with PD-L1 hinge and transmembrane domain, and cytoplasmic domain from Trop-2 (tBCMA; middle panel). Unstained control is shown in the right panel.

FIG. 3 shows CD19 CAR and BCMA staining of cells enriched using anti-APC magnetic beads.

FIG. 4 shows percent change in live cells for primary T cells (transduced with BCL6 and BCL2L1) transduced with BCMA fusion proteins, treated with the indicated concentrations of belantamab mafodotin.

FIG. 5 shows BCMA staining (horizontal axes) of Jurkat cells transduced with a lentiviral vector expressing either: BCMA extracellular domain alone, BCMA extracellular domain fused with cytoplasmic domain from CD317, or BCMA extracellular domain fused with cytoplasmic domain from CD3γ, each treated with the shown concentrations of belantamab mafodotin (0, 12.5, or 25 μg).

FIG. 6 shows CD30 and CD69 staining for primary T cells transfected with BCL6 and BCL2L1.

FIG. 7 shows percent change in live cells for primary T cells transfected with BCL6 and BCL2L1 treated with the shown concentrations of brentuximab vedotin for the indicated time.

FIG. 8 shows a schematic of a construct encoding a CD19 CAR and both 1) Her2 domain 4 and 2) CD30 extracellular domain fused with BCMA cytoplasmic tail.

FIG. 9 shows CD30 staining (left panel) and Her2 staining (middle panel) of 293T cells transduced with the construct shown in FIG. 8. The right panel shows live cell number change on day 4 after treatment with the indicated concentrations of brentuximab.

FIG. 10 shows a schematic of a construct encoding a CD19 CAR and both 1) Her2 domain 4 and 2) truncated EGFR.

FIG. 11A shows Her2 and CD19 CAR staining of Jurkat T cells transduced with the construct shown in FIG. 10. FIG. 11B shows percent live cell change following treatment of the cells with the indicated concentrations of trastuzamab (Herceptin®).

FIG. 12 shows a schematic of a construct encoding a CD19 CAR and both 1) Her2 domain 4 and 2) Trop2.

FIG. 13 shows CD19 CAR and Trop2 staining of 293T cells transfected with the construct shown in FIG. 12.

FIG. 14 shows a schematic of a construct encoding a CD19 CAR and both 1) Her2 domain 4 and 2) truncated EGFR.

FIG. 15 shows CD19 CAR and EGFR staining (Cetuximab) of 293T cells transfected with the construct shown in FIG. 14.

FIG. 16A shows tBCMA Safety Switch having in vitro efficacy. T cells transfected with the tBCMA safety switch, BCL6, and BCL2L1 are the cluster of bar graphs on the left, T cells transfected with the tBCMA safety switch, a CD19 CAR, BCL6, and BCL2L1 are the cluster of bar graphs in the middle, and control non-transfected Raji cells are the cluster of bar graphs on the right.

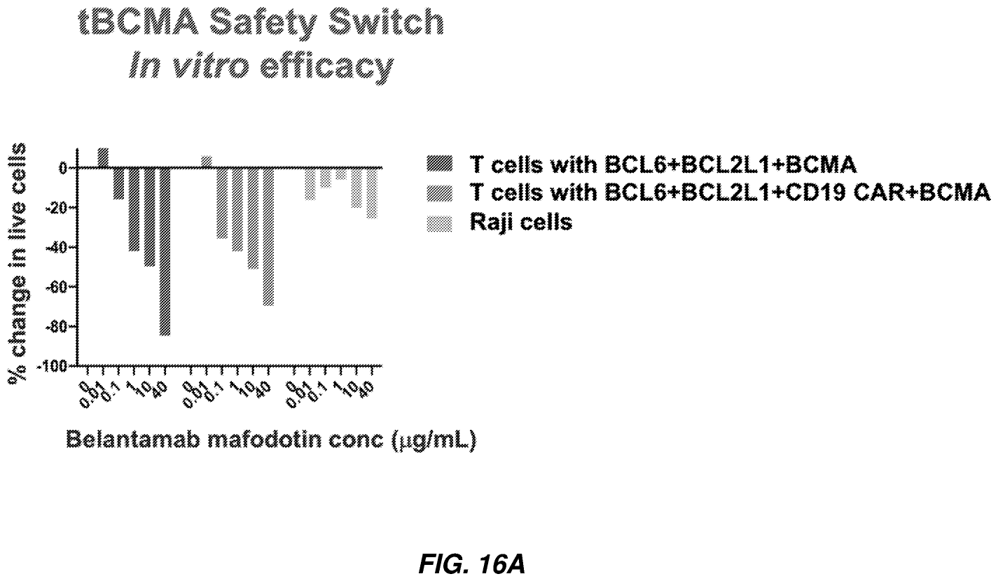

FIG. 16B shows tBCMA Safety Switch having in vivo efficacy.

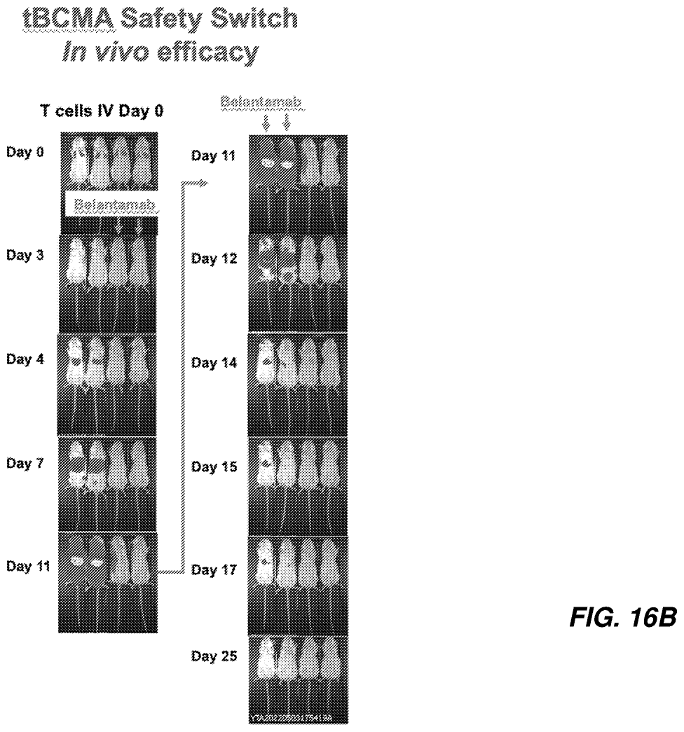

### DETAILED DESCRIPTION

Aspects of the present disclosure are directed to polypeptides useful in detecting, isolating, depleting, and/or purifying cells. Accordingly, certain aspects of the disclosure are directed to chimeric polypeptides comprising, for example, an extracellular domain from BCMA, an extracellular domain from Trop-2, an extracellular domain from CD30, an extracellular domain from EGFR (e.g., domain III and a portion of domain IV from EGFR), and/or an extracellular domain from Her2 (e.g., domain IV from HER2). Chimeric polypeptides of the disclosure may also comprise one or more additional domains or regions, such as a signal peptide, a hinge, a transmembrane region, and/or one or more intracellular regions. Also disclosed are cells (e.g., therapeutic cells) comprising one or more polypeptides (e.g., chimeric polypeptides) of the present disclosure, as well as methods for detecting, isolating, depleting, and/or purifying such cells.

In particular embodiments, the chimeric polypeptides are utilized for one or more specific purposes associated with cell therapies, for example. In specific embodiments, the chimeric polypeptides are utilized for direct or indirect control of the use of particular therapeutic cells and allow for monitoring of a cell therapy, detection of cells in a cell therapy, isolation of cells for a cell therapy, and/or termination of a cell therapy at a desired event and/or time. In particular embodiments, the chimeric polypeptides are utilized as a transduction marker, as a safety switch, or both. In some cases, the chimeric polypeptides encompassed herein are used as a transduction marker or selection marker in the cells, but another safety switch controls inhibition of the cells. In other cases, the chimeric polypeptide is used as a safety switch in the cells optionally in addition to another safety switch in the same cell.

The safety switch chimeric polypeptide may be employed in transduced/transfected cells for the purpose of eliciting death for the cells when needed. The cells that utilize the chimeric polypeptides may comprise one or more different chimeric polypeptides that are safety switches. In some embodiments, the safety switch chimeric polypeptide is utilized as a “suicide gene” that upon administration of a prodrug or other agent, effects transition of a gene product to a compound that kills its host cell. In other embodiments, the safety switch chimeric polypeptide is utilized as a suicide gene that encodes a gene product that is, when desired, targeted by an agent (such as an antibody) that targets the suicide gene product.

In some cases, an individual receives cell therapy wherein the cells express the chimeric polypeptide. The individual may be subject to utilization of one or more agents that bind the extracellular domain of the chimeric polypeptide when the individual receiving the cell therapy and/or having received the cell therapy shows one or more symptoms of one or more adverse events, such as cytokine release syndrome, neurotoxicity, anaphylaxis/allergy, and/or on-target/off tumor toxicities (as examples) or is considered at risk for having the one or more symptoms, including imminently. The use of the agent that binds the chimeric polypeptide may be part of a planned protocol for a therapy or may be used only upon a recognized need for its use. In some cases, the cell therapy is terminated by use of agent(s) that targets the extracellular domain of the chimeric polypeptide because the therapy is no longer required.

Utilization of the chimeric polypeptide as a safety switch may be instigated upon onset of at least one adverse event for the individual, and that adverse event may be recognized by any means, including upon routine monitoring that may or may not be continuous from the beginning of the cell therapy. The adverse event(s) may be detected upon examination and/or testing. In cases wherein the individual has cytokine release syndrome (which may also be referred to as cytokine storm), the individual may have elevated inflammatory cytokine(s) (merely as examples: interferon-gamma, granulocyte macrophage colony-stimulating factor, IL-10, IL-6 and TNF-alpha); fever; fatigue; hypotension; hypoxia, tachycardia; nausea; capillary leak; cardiac/renal/hepatic dysfunction; or a combination thereof, for example. In cases wherein the individual has neurotoxicity, the individual may have confusion, delirium, aphasia, and/or seizures. In some cases, the individual is tested for a marker associated with onset and/or severity of cytokine release syndrome, such as C-reactive protein, IL-6, TNF-alpha, and/or ferritin.

In particular embodiments, the cell therapy may encompass one or more vectors that encode one or more heterologous proteins, and such heterologous protein(s) may be a composition that renders the cells therapeutic. In some embodiments, the vector that encodes the one or more heterologous proteins encodes one or more safety switches. The safety switch may or may not be on the same vector as a CAR, for example. In cases wherein the safety switch is present on the same vector as a CAR, the safety switch and the CAR may be separated by an IRES or 2A element, for example.

### I. Polypeptides

Aspects of the present disclosure relate to polypeptides, including chimeric polypeptides, and methods of use thereof. As used herein, a “protein” or “polypeptide” refers to a molecule comprising at least five amino acid residues. As used herein, the term “wild-type” refers to the endogenous version of a molecule that occurs naturally in an organism. In some embodiments, wild-type versions of a protein or polypeptide are employed, however, in many embodiments of the disclosure, a modified protein or polypeptide is employed. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. In some embodiments, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects.

Where a protein is specifically mentioned herein, it is in general a reference to a native (wild-type) or recombinant (modified) protein or, optionally, a protein in which any signal sequence has been removed. The protein may be isolated directly from the organism of which it is native, produced by recombinant DNA/exogenous expression methods, or produced by solid-phase peptide synthesis (SPPS) or other in vitro methods. In particular embodiments, there are isolated nucleic acid segments and recombinant vectors incorporating nucleic acid sequences that encode a polypeptide (e.g., an antibody or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

In certain embodiments the size of a protein or polypeptide (wild-type or modified) may comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 amino acid residues or greater, and any range derivable therein, or derivative of a corresponding amino sequence described or referenced herein. It is contemplated that polypeptides may be mutated by truncation, rendering them shorter than their corresponding wild-type form, also, they might be altered by fusing or conjugating a heterologous protein or polypeptide sequence with a particular function (e.g., for targeting or localization, for enhanced immunogenicity, for purification purposes, etc.). As used herein, the term “domain” refers to any distinct functional or structural unit of a protein or polypeptide, and generally refers to a sequence of amino acids with a structure or function recognizable by one skilled in the art.

The polypeptides, proteins, or polynucleotides encoding such polypeptides or proteins of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107,108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids or nucleotides, or any range derivable therein, of SEQ ID NOs:1-48.

In some embodiments, the protein, polypeptide, or nucleic acid may comprise amino acids or nucleotides, respectively, 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000, (or any derivable range therein) of SEQ ID NOs:1-48.

In some embodiments, the protein, polypeptide, or nucleic acid may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104,105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000, (or any derivable range therein) contiguous amino acids or nucleotides, respectively, of SEQ ID NOs:1-48.

In some embodiments, the polypeptide, protein, or nucleic acid may comprise at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein) contiguous amino acids or nucleotides, respectively, of SEQ ID NOs:1-48 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with one of SEQ ID NOS:1-48, respectively.

In some aspects there is a nucleic acid molecule or polypeptide starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110,111, 112, 113, 114, 115, 116, 117, 118, 119, 120,121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 of any of SEQ ID NOS:1-48 and comprising at least, at most, or exactly 2,3,4,5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein) contiguous amino acids or nucleotides of any of SEQ ID NOS:1-48.

The polypeptides and peptides of the disclosure may include 1, 2, 3, 4, 5, 6, 7, 8, 9, or 10 (or any derivable range therein) or more variant amino acids substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with at least, or at most 3, 4, 5, 6, 7, 8, 9, 10, 11, 12, 13, 14, 15, 16, or 17 contiguous amino acids or nucleotides, or any range derivable therein, of any of SEQ ID NOs:1-48.

In some embodiments, the peptide, polypeptide, or nucleic acid may comprise amino acids or nucleotides 1 to 2, 3, 4, 5, 6, 7, 8, 9, 10, 11, 12, 13, 14, 15, 16, 17, 18, 19, 20, 21, 22, 23, 24, or 25 (or any derivable range therein) of any of SEQ ID NOs:1-48.

In some embodiments, the peptide, polypeptide, or nucleic acid may comprise 1, 2, 3, 4, 5, 6, 7, 8, 9, 10, 11, 12, 13, 14, 15, 16, 17, 18, 19, 20, 21, 22, 23, 24, or 25 (or any derivable range therein) contiguous amino acids of SEQ ID NOs:1-48.

In some embodiments, the peptide, polypeptide, or nucleic acid may comprise, may comprise at least, or may comprise at most 1, 2, 3, 4, 5, 6, 7, 8, 9, 10, 11, 12, 13, 14, 15, 16, 17, 18, 19, 20, 21, 22, 23, 24, or 25 (or any derivable range therein) contiguous amino acids or nucleotides of any of SEQ ID NOs:1-48 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with one of SEQ ID NOs:1-48.

In some aspects there is a peptide, polypeptide, or nucleic acid starting at position 1, 2, 3, 4, 5, 6, 7, 8, 9, or 10 of any of SEQ ID NOs:1-48 and comprising, comprising at least, or comprising at most 2, 3, 4, 5, 6, 7, 8, 9, 10, 11, 12, 13, 14, 15, 16, 17, 18, 19, 20, 21, 22, 23, 24, or 25 (or any derivable range therein) contiguous amino acids or nucleotides of any of SEQ ID NOs:1-48.

The peptide, polypeptide, or nucleic acid may comprise, may comprise at least, or may comprise at most 1, 2, 3, 4, 5, 6, 7, 8, 9, or 10 (or any derivable range therein) amino acid or nucleotide substitutions relative to SEQ ID NOs:1-48. The peptide, polypeptide, or nucleic acid may comprise, may comprise at least, or may comprise at most 1, 2, 3, 4, 5, 6, 7, 8, 9, or 10 (or any derivable range therein) amino acid or nucleotide substitutions, and the substitution(s) may be at position 1, 2, 3, 4, 5, 6, 7, 8, 9, 10, 11, 12, 13, 14, 15, 16, 17, 18, 19, 20, 21, 22, 23, 24, and/or 25 relative to SEQ ID NOs:1-48. For amino acid substitutions, the substitution (e.g., at position(s) 1, 2, 3, 4, 5, 6, 7, 8, 9, 10, 11, 12, 13, 14, 15, 16, 17, 18, 19, 20, 21, 22, 23, 24, and/or 25) may be with an alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine. For nucleic acid substitutions, the substitution (e.g., at position(s) 1, 2, 3, 4, 5, 6, 7, 8, 9, 10, 11, 12, 13, 14, 15, 16, 17, 18, 19, 20, 21, 22, 23, 24, and/or 25) may be with a guanine, cytosine, adenine, thymine, uracil, or other nucleotide.

The nucleotide as well as the protein, polypeptide, and peptide sequences for various genes have been previously disclosed, and may be found in the recognized computerized databases. Two commonly used databases are the National Center for Biotechnology Information's GENBANK® and GENPEPT® databases (on the World Wide Web at ncbi.nlm.nih.gov/) and The Universal Protein Resource (UniProt; on the World Wide Web at uniprot.org). The coding regions for these genes may be amplified and/or expressed using the techniques disclosed herein or as would be known to those of ordinary skill in the art.

It is contemplated that in compositions of the disclosure, in some embodiments, there is between about 0.001 mg and about 10 mg of total polypeptide, peptide, and/or protein per ml. The concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein).

### A. Chimeric Polypeptides

As disclosed herein, a “chimeric polypeptide” describes any polypeptide having regions, domains, or other portions that are derived from two or more different polypeptides. For example, one example of a chimeric polypeptide is a polypeptide having a first domain derived from a first protein and a second domain derived from a second protein. A region or domain is described as “from” or “derived from” a protein or polypeptide when the region or domain has the same sequence as at least a portion of the polypeptide. Thus, for example, a hinge region from PDL1 (also “derived from” PDL1) describes a hinge region that has the same sequence as at least a portion of a hinge region of the PDL1 protein. A region or domain “derived from” a protein or polypeptide may also be described as a protein or polypeptide region or domain; for example a transmembrane domain derived from CD30 may also be described as a “CD30 transmembrane domain.” A chimeric polypeptide may comprise regions from at least 2, 3, 4, 5, 6, or more different polypeptides. Example chimeric polypeptides include chimeric antigen receptors (CARs) and other chimeric cell surface polypeptides. In some embodiments, a chimeric polypeptide of the disclosure is a polypeptide comprising an extracellular domain from BCMA and one or more additional regions or domains derived from a protein that is not BCMA. In some embodiments, a chimeric polypeptide of the disclosure is a polypeptide comprising an extracellular domain from CD30 and one or more additional regions or domains derived from a protein that is not CD30. In some embodiments, a chimeric polypeptide of the disclosure is a polypeptide comprising an extracellular domain from Her2 and one or more additional regions or domains derived from a protein that is not Her2. In some embodiments, a chimeric polypeptide of the disclosure is a polypeptide comprising an extracellular domain from EGFR and one or more additional regions or domains derived from a protein that is not EGFR. Chimeric polypeptides of the disclosure may comprise, for example, a signal peptide (e.g., a tissue-type plasminogen activator (tPA) signal peptide, a CD8a signal peptide, or a GM-CSFRα signal peptide), an extracellular domain (e.g., an extracellular domain from BCMA, an extracellular domain from CD30, an extracellular domain from Her2, or an extracellular domain from EGFR), a hinge domain (e.g., a hinge domain from PDL1 or a hinge domain from CD8), a transmembrane domain (e.g., a transmembrane domain from PDL1, a transmembrane domain from CD8, a transmembrane domain from CD30, or a transmembrane domain from Her2), an intracellular region (e.g., an intracellular region from CD8, an intracellular region from CD317, an intracellular region from CD3γ, or an intracellular region from BCMA), or any combination thereof. Any one or more of these regions or domains may be excluded from certain embodiments of the disclosure.

In some embodiments, disclosed herein is a chimeric polypeptide comprising an extracellular domain from BCMA, a hinge region, a transmembrane domain, and an intracellular region. The chimeric polypeptide may comprise an extracellular domain having an amino acid sequence that is, is at least, or is at most 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) identical to an extracellular domain from the BCMA protein. In some embodiments, the extracellular domain from BCMA is, is at least, or is at most 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) identical to SEQ ID NO:19.

A chimeric polypeptide of the disclosure may comprise a hinge region from, for example, CD8a, PDL1, IgG4, IgG1, or CD34. In some embodiments, the hinge region has an amino acid sequence that is, is at least, or is at most 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) identical to a CD8a hinge, a PDL1 hinge, an IgG4 hinge, an IgG1 hinge, or a CD34 hinge. In some embodiments, the hinge region is, is at least, or is at most 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) identical to a PDL1 hinge. In some embodiments, the hinge region is, is at least, or is at most 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) identical to SEQ ID NO:23. In some embodiments, the hinge region comprises SEQ ID NO:23. In some embodiments, the hinge region is, is at least, or is at most 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) identical to a CD8a hinge. In some embodiments, the hinge region is a hinge region from CD8a. In some embodiments, the hinge region is, is at least, or is at most 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) identical to SEQ ID NO:24. In some embodiments, the hinge region comprises SEQ ID NO:24. In some embodiments, the hinge region is, is at least, or is at most 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) identical to SEQ ID NO:37. In some embodiments, the hinge region comprises SEQ ID NO:37.

A chimeric polypeptide of the disclosure may comprise a transmembrane region from one or more transmembrane proteins. A chimeric polypeptide of the disclosure may comprise a transmembrane region from, for example, 4-1BB/CD137, an alpha chain of a T cell receptor, a beta chain of a T cell receptor, CD2, CD3 delta, CD3 epsilon, CD3 gamma, CD4, CD7, CD8a, CD80, CD9, CD11a (ITGAL), CD11b (ITGAM), CD11c (ITGAX), CD11d (ITGAD), CD16, CD18 (ITGB2), CD19 (B4), CD22, CD27 (TNFRSF7), CD28, CD29 (ITGB1), CD30 (TNFRSF8), CD33, CD37, CD40 (TNFRSF5), CD48 (SLAMF2), CD49a (ITGA1), CD49d (ITGA4), CD49f (ITGA6), CD64, CD66a (CEACAM1), CD66b (CEACAM8), CD66c (CEACAM6), CD66d (CEACAM3), CD66e (CEACAM5), CD69 (CLEC2), CD79A (B-cell antigen receptor complex-associated alpha chain), CD79B (B-cell antigen receptor complex-associated beta chain), CD80, CD84 (SLAMF5), CD86, CD96 (Tactile), CD100 (SEMA4D), CD103 (ITGAE), CD123, CD134 (OX40), CD137 (4-1BB), CD150 (SLAMF1), CD154, CD158A (KIR2DL1), CD158B1 (KIR2DL2), CD158B2 (KIR2DL3), CD158C (KIR3DP1), CD158D (KIRDL4), CD158F1 (KIR2DL5A), CD158F2 (KIR2DL5B), CD158K (KIR3DL2), CD160 (BY55), CD162 (SELPLG), CD226 (DNAM1), CD229 (SLAMF3), CD244 (SLAMF4), CD247 (CD3-zeta), CD258 (LIGHT), CD268 (BAFFR), CD270 (TNFSF14), CD272 (BTLA), CD276 (B7-H3), CD279 (PD-1), CD314 (NKG2D), CD319 (SLAMF7), CD335 (NK-p46), CD336 (NK-p44), CD337 (NK-p30), CD352 (SLAMF6), CD353 (SLAMF8), CD355 (CRTAM), CD357 (TNFRSF18), inducible T cell co-stimulator (ICOS), LFA-1 (CD11a/CD18), NKG2C, DAP-10, ICAM-1, NKp80 (KLRF1), IL-2R beta, IL-2R gamma, IL-7R alpha, LFA-1, SLAMF9, LAT, GADS (GrpL), SLP-76 (LCP2), PAG1/CBP, a CD83 ligand, Fc gamma receptor, an MHC class 1 molecule, an MHC class 2 molecule, or a TNF receptor protein. In some embodiments, the transmembrane domain is an alpha or beta chain of the T cell receptor or a transmembrane domain from CD28, CD3e, CD45, CD4, CD5, CD8, CD9, CD16, CD22, CD33, CD37, CD64, CD80, CD86, CD123, CD134, CD137 or CD154. In some embodiments, the transmembrane domain is, is at least, or is at most 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) identical to a CD8a transmembrane domain. In some embodiments, the transmembrane domain is a transmembrane domain from CD8a. In some embodiments, the transmembrane domain is, is at least, or is at most 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) identical to SEQ ID NO:26. In some embodiments, the transmembrane domain comprises SEQ ID NO:26. In some embodiments, the transmembrane domain is, is at least, or is at most 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) identical to a CD30 transmembrane domain. In some embodiments, the transmembrane domain is a transmembrane domain from CD30. In some embodiments, the transmembrane domain is, is at least, or is at most 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) identical to SEQ ID NO:27. In some embodiments, the transmembrane domain comprises SEQ ID NO:27. In some embodiments, the transmembrane domain is, is at least, or is at most 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) identical to a Her2 transmembrane domain. In some embodiments, the transmembrane domain is a transmembrane domain from Her2. In some embodiments, the transmembrane domain is, is at least, or is at most 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) identical to SEQ ID NO:28. In some embodiments, the transmembrane domain comprises SEQ ID NO:28.

A chimeric polypeptide of the disclosure may comprise an intracellular region (also “cytoplasmic region”) from or more transmembrane proteins. As used herein, an “intracellular region,” describes a region of a polypeptide that, when the polypeptide is expressed on the surface of a cell, is present on the intracellular (or “cytoplasmic”) side of the cell surface. In some embodiments, the intracellular region does not comprise a signaling domain (i.e., is not capable of transmitting or transducing any intracellular signal). In some embodiments, the intracellular region is not from BCMA. In some embodiments, the intracellular region is from BCMA. In some embodiments, the intracellular region is from CD317. In some embodiments, the intracellular region is from CD3γ. In some embodiments, the intracellular region comprises or comprises at most 20, 19, 18, 17, 16, 15, 14, 13, 12, 11, 10, 9, 8, 7, 6, 5, 4, 3, or 2 amino acid residues, or any range derivable therein. In some embodiments, the intracellular region comprises the sequence RLR (SEQ ID NO:29). In some embodiments, the intracellular region consists of the sequence RLR (SEQ ID NO:29). In some embodiments, the intracellular region comprises the sequence LYCWVR (SEQ ID NO:30). In some embodiments, the intracellular region consists of the sequence LYCWVR (SEQ ID NO:30). In some embodiments, is, is at least, or is at most 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) identical to SEQ ID NO:31. In some embodiments, the intracellular region comprises SEQ ID NO:31. In some embodiments, is, is at least, or is at most 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) identical to SEQ ID NO:32. In some embodiments, the intracellular region comprises SEQ ID NO:32. In some embodiments, is, is at least, or is at most 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) identical to SEQ ID NO:33. In some embodiments, the intracellular region comprises SEQ ID NO:33.

In some embodiments, a chimeric polypeptide of the disclosure comprises a signal peptide. In some embodiments, the signal peptide is a tissue-type plasminogen activator (tPA) signal peptide. In some embodiments, the signal peptide is, is at most, or is at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) identical to SEQ ID NO:34. In some embodiments, the signal peptide comprises SEQ ID NO:34. In some embodiments, the signal peptide is a CD8a signal peptide. In some embodiments, the signal peptide is, is at most, or is at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) identical to SEQ ID NO:35. In some embodiments, the signal peptide comprises SEQ ID NO:35. In some embodiments, the signal peptide is a GM-CSFRα signal peptide. In some embodiments, the signal peptide is, is at most, or is at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) identical to SEQ ID NO:36. In some embodiments, the signal peptide comprises SEQ ID NO:36.

### B. Sequences

The amino acid sequence of certain polypeptides, including chimeric polypeptides and portions, regions, and domains thereof, are provided in Table 1.

TABLE 1

| | SEQ ID | |
|---|---|---|
| Name | NO: | Sequence |
| BCMA extracellular | 1 | MLQMAGQCSQNEYFDSLLHACIPCQ |
| domain-PDL1 hinge & | | LRCSSNTPPLTCQRYCNASVTNSVK |
| TM (polypeptide) | | GTNAHPPNERTHLVILGAILLCLGV |
| | | ALTFIFRLR |
| BCMA extracellular | 2 | ATGCTCCAGATGGCTGGCCAGTGTT |
| domain-PDL1 hinge & | | CGCAGAACGAGTACTTCGACAGTCT |
| TM (DNA) | | TCTACACGCCTGTATTCCTTGTCAG |
| | | CTGCGTTGCTCTAGCAACACGCCCC |
| | | CACTGACCTGCCAGCGTTACTGCAA |
| | | TGCLTCCGTCACCAACTCCGTGAAG |
| | | GGCACCAACGCGCATCCGCCCAACG |
| | | AGCGCACACACCTGGTGATCTTGGG |
| | | TGCCATCCTGCTGTGCCTGGGGGTG |
| | | GCACTCACTTTTATCTTCCGCCTGC |
| | | GGTAA |
| BCMA extracellular | 3 | MDAMKRGLCCVLLLCGAVFVSPSQE |
| domain-CD8 hinge & TM | | IHARFRRGARMLQMAGQCSQNEYFD |
| (polypeptide) | | SLLHACIPCQLRCSSNTPPLTCQRY |
| | | CNASVTNSVKGTNAPTIASQPLSLR |
| | | PEACRPAAGGAVHTRGLDFACDIYI |
| | | WAPLAGTCGVLLLSLVITLYCWVR |
| BCMA extracellular | 4 | ATGGATGCCATGAAGCGCGGTCTGT |
| domain-CD8 hinge & TM | | GCTGCGTGTTGCTGCTGTGCGGGGC |
| (DNA) | | CGTGTTCGTCTCACCCTCCCAGGAG |
| | | ATCCACGCCCGCTTCAGACGTGGTG |
| | | CCCGTATGCTGCAGATGGCTGGCCA |
| | | GTGCTCTCAGAACGAGTACTTCGAC |
| | | AGCCTTTTGCATGCTTGTATTCCGT |
| | | GCCAGCTGAGGTGTTCGAGCAACAC |
| | | GCCCCCACTCACCTGTCAGCGTTAC |
| | | TGCAATGCGTCCGTAACCAACTCCG |
| | | TGAAGGGCACTAACGCGCCTACCAT |
| | | CGCGAGTCAACCGCTGTCTCTGCGG |
| | | CCCGAGGCCTGCCGACCCGCGGCCG |
| | | GCGGCGCTGTTCACACTCGCGGCCT |
| | | GGACTTTGCATGCGACATCTATATT |
| | | TGGGCACCTCTTGCTGGAACATGCG |
| | | GGGTGCTGCTCCTCTCCCTAGTGAT |
| | | CACCCTGTACTGTTGGGTCCGCTAA |
| BCMA extracellular | 5 | MLQMAGQCSQNEYFDSLLHACIPCQ |
| domain-PDL1 hinge & | | LRCSSNTPPLTCQRYCNASVTNSVK |
| TM-CD317 cytoplasmic | | GTNAHPPNERTHLVILGAILLCLGV |
| (polypeptide) | | ALTFIFRASTSYDYCRVPG |
| BCMA extracellular | 6 | ATGCTCCAGATGGCTGGCCAGTGTT |
| domain-PDL1 hinge & | | CGCAGAACGAGTACTTCGACAGTCT |
| TM-CD317 cytoplasmic | | TCTACACGCCTGTATTCCTTGTCAG |
| (DNA) | | CTGCGTTGCTCTAGCAACACGCCCC |
| | | CACTGACCTGCCAGCGTTACTGCAA |
| | | TGCTTCCGTCACCAACTCCGTGAAG |
| | | GGCACCAACGCGCATCCGCCCAACG |
| | | AGCGCACACACCTGGTGATCTTGGG |
| | | TGCCATCCTGCTGTGCCTGGGGGTG |
| | | GCACTCACTTTTATCTTCCGCGCAT |
| | | CCACGAGTTACGATTACTGTCGAGT |
| | | TCCAGGG |
| BCMA extracellular | 7 | MLQMAGQCSQNEYFDSLLHACIPCQ |
| domain-PDL1 hinge & | | LRCSSNTPPLTCQRYCNASVTNSVK |
| TM-CD3γ cytoplasmic | | GTNAHPPNERTHLVILGAILLCLGV |
| (polypeptide) | | ALTFIFRASDKQTLLPG |
| BCMA extracellular | 8 | ATGCTCCAGATGGCTGGCCAGTGTT |
| domain-PDL1 hinge & | | CGCAGAACGAGTACTTCGACAGTCT |
| TM-CD3γ cytoplasmic | | TCTACACGCCTGTATTCCTTGTCAG |
| (DNA) | | CTGCGTTGCTCTAGCAACACGCCCC |
| | | CACTGACCTGCCAGCGTTACTGCAA |
| | | TGCTTCCGTCACCAACTCCGTGAAG |
| | | GGCACCAACGCGCATCCGCCCAACG |
| | | AGCGCACACACCTGGTGATCTTGGG |
| | | TGCCATCCTGCTGTGCCTGGGGGTG |
| | | GCACTCACTTTTATCTTCCGCGCAT |
| | | CCGACAAGCAGACCCTTCTCCCTGG |
| | | GTGA |
| CD30 extracellular domain | 9 | MRVLLAALGLLFLGALRAFPQDRPF |
| and TM domain fused with | | EDTCHGNPSHYYDKAVRRCCYRCPM |
| BCMA cytoplasmic tail | | GLFPTQQPQRPTDCRKQCEPDYYLD |
| (polypeptide) | | EADRCTACVTCSRDDLVEKTPCAWN |
| | | SSRVCECRPGMFCSTSAVNSCARCF |
| | | FHSVCPAGMIVKFPGTAQKNTVCEP |
| | | ASPGVSPACASPENCKEPSSGTIPQ |
| | | AKPTPVSPATSSASTMPVRGGTRLA |
| | | QEAASKLTRAPDSPSSVGRPSSDPG |
| | | LSPTQPCPEGSGDCRKQCEPDYYLD |
| | | EAGRCTACVSCSRDDLVEKTPCAWN |
| | | SSRTCECRPGMICATSATNSCARCV |
| | | PYPICAAETVTKPQDMAEKDTTFEA |
| | | PPLGTQPDCNPTPENGEAPASTSPT |
| | | QSLLVDSQASKTLPIPTSAPVALSS |
| | | TGKPVLDAGPVLFWVILVLVVVVGS |
| | | SAFLLCHRKINSEPLKDEFKNTGSG |
| | | LLGMANIDLEKSRTGDEIILPRGLE |
| | | YTVEECTCEDCIKSKPKVDSDHCFP |
| | | LPAMEEGATILVTTKTNDYCKSLPA |
| | | ALSATEIEKSISAR |
| CD30 extracellular domain | 10 | ATGCGAGTCCTCCTGGCCGCGTTGG |
| and TM domain fused with | | GGCTCTTGTTCCTTGGGGCACTTCG |
| BCMA cytoplasmic tail | | AGCCTTTCCACAGGATCGACCTTTT |
| (DNA) | | GAAGATACTTGTCACGGAAACCCTT |
| | | CTCACTACTACGATAAAGCGGTCCG |
| | | ACGATGCTGCTACCGATGCCCTATG |
| | | GGACTTTTCCCGACGCAGCAATGCC |
| | | CACAGCGGCCTACGGACTGTAGAAA |
| | | GCAATGCGAGCCGGACTACTATCTG |
| | | GACGAAGCAGATCGGTGTACTGCCT |
| | | GCGTAACATGCTCCCGGGATGACCT |
| | | GGTGGAAAAGACCCCCTGCGCTTGG |
| | | AACTCCAGTAGAGTATGCGAATGCC |
| | | GACCAGGGATGTTCTGCAGCACGAG |
| | | CGCAGTTAACTCATGTGCCAGGTGT |
| | | TTTTTCCATTCTGTATGTCCTGCCG |
| | | GGATGATTGTTAAGTTCCCAGGTAC |
| | | AGCTCAAAAGAACACGGTATGCGAA |
| | | CCCGCTAGTCCGGGAGTTTCCCCGG |
| | | CCTGCGCCAGCCCAGAGAATTGCAA |
| | | GGAGCCGTCTAGCGGTACAATCCCA |
| | | CAAGCTAAGCCGACGCCGGTCAGCC |
| | | CGGCGACTTCATCCGCCTCAACAAT |
| | | GCCCGTCCGGGGTGGGACAAGACTC |
| | | GCGCAGGAAGCGGCTAGCAAGTTGA |
| | | CACGAGCCCCCGATTCCCCTTCAAG |
| | | TGTAGGCAGACCTAGTTCCGACCCT |
| | | GGCCTTAGCCCAACGCAACCCTGCC |
| | | CAGAGGGATCCGGAGACTGTCGGAA |
| | | ACAATGCGAGCCCGACTATTACTTG |
| | | GATGAAGCCGGTCGCTGTACCGCAT |
| | | GTGTGTCCTGCAGCCGCGATGACCT |
| | | GGTCGAGAAAACACCATGTGCATGG |
| | | AATAGCAGTCGCACCTGCGAGTGCC |
| | | GACCGGGAATGATCTGTGCCACCTC |
| | | AGCAACCAACTCTTGTGCCAGGTGC |
| | | GTACCATATCCCATCTGCGCGGCTG |
| | | AGACCGTAACAAAACCTCAAGACAT |
| | | GGCCGAAAAAGACACCACTTTCGAA |
| | | GCGCCGCCTCTGGGCACTCAACCAG |
| | | ACTGCAATCCTACGCCAGAAAACGG |
| | | GGAAGCACCCGCGTCAACCTCCCCC |
| | | ACACAATCTTTGCTCGTAGACTCTC |
| | | AGGCTTCCAAAACACTGCCAATACC |
| | | AACTTCCGCTCCGGTGGCTCTGAGC |
| | | TCTACCGGCAAACCCGTGCTTGACG |
| | | CCGGGCCAGTCCTGTTCTGGGTTAT |
| | | ATTGGTGCTCGTAGTCGTAGTAGGC |
| | | TCCAGCGCCTTTCTGCTCTGTCACC |
| | | GGAAGATCAATTCCGAACCTTTGAA |
| | | AGACGAGTTTAAGAACACCGGGAGT |
| | | GGCCTCCTCGGAATGGCTAATATCG |
| | | ACTTGGAGAAGAGCCGCACTGGGGA |
| | | CGAAATCATTTTGCCTCGCGGGCTT |
| | | GAATACACAGTCGAAGAGTGCACGT |
| | | GTGAAGACTGCATTAAATCAAAACC |
| | | GAAGGTGGACAGCGATCATTGTTTC |
| | | CCCTTGCCCGCTATGGAAGAAGGTG |
| | | CAACTATCCTCGTAACAACCAAAAC |
| | | TAACGATTATTGTAAAAGCCTCCCG |
| | | GCGGCTCTCTCTGCGACGGAAATAG |
| | | AAAAATCAATCTCTGCAAGG |
| Domain IV and | 11 | MALPVTALLLPLALLLHAARPLACH |
| Transmembrane domain of | | QLCARGHCWGPGPTQCVNCSQFLRG |
| HER2-with CD8α signal | | QECVEECRVLQGLPREYVNARHCLP |
| peptide (polypeptide) | | CHPECQPQNGSVTCFGPEADQCVAC |
| | | AHYKDPPFCVARCPSGVKPDLSYMP |
| | | IWKFPDEEGACQPCPINCTHSCVDL |
| | | DDKGCPAEQRASPLTSIISAVVGIL |
| | | LVVVLGVVFGILIKR |
| Domain IV and | 12 | ATGGCCCTGCCAGTGACCGCCCTGC |
| Transmembrane domain of | | TGCTGCCACTGGCACTGCTGCTGCA |
| HER2-with CD8α signal | | CGCAGCAAGGCCACTCGCTTGTCAC |
| peptide (DNA) | | CAGCTGTGCGCTCGCGGCCACTGTT |
| | | GGGGCCCCGGTCCTACCCAGTGCGT |
| | | CAACTGTTCGCAGTTCCTAAGAGGG |
| | | CAGGAGTGCGTGGAGGAATGCCGTG |
| | | TGCTGCAGGGTCTGCCACGCGAGTA |
| | | CGTGAACGCCCGTCACTGTCTGCCA |
| | | TGTCACCCCGAGTGCCAGCCCCAGA |
| | | ACGGGAGCGTGACCTGCTTTGGACC |
| | | GGAGGCGGACCAGTGCGTGGCTTGT |
| | | GCCCATTACAAGGATCCACCGTTCT |
| | | GCGTGGCGAGGTGTCCCTCTGGCGT |
| | | GAAGCCCGATCTGAGCTACATGCCT |
| | | ATATGGAAATTTCCGGACGAGGAAG |
| | | GGGCTTGTCAGCCTTGCCCGATCAA |
| | | CTGTACTCATTCCTGCGTGGACTTG |
| | | GATGACAAGGGATGCCCGGCGGAGC |
| | | AGCGCGCGTCCCCGTTGACCAGCAT |
| | | TATCTCCGCAGTGGTGGGCATCCTG |
| | | TTGGTCGTCGTTCTGGGCGTCGTGT |
| | | TCGGTATTCTCATCAAGCGG |
| Full length WT Trop2 | 13 | MARGPGLAPPPLRLPLLLLVLAAVT |
| (polypeptide) | | GHTAAQDNCTCPTNKMTVCSPDGPG |
| | | GRCQCRALGSGMAVDCSTLTSKCLL |
| | | LKARMSAPKNARTLVRPSEHALVDN |
| | | DGLYDPDCDPEGRFKARQCNQTSVC |
| | | WCVNSVGVRRTDKGDLSLRCDELVR |
| | | THHILIDLRHRPTAGAFNHSDLDAE |
| | | LRRLFRERYRLHPKFVAAVHYEQPT |
| | | IQIELRQNTSQKAAGDVDIGDAAYY |
| | | FERDIKGESLFQGRGGLDLRVRGEP |
| | | LQVERTLIYYLDEIPPKFSMKRLTA |
| | | GLIAVIVVVVVALVAGMAVLVITNR |
| | | RKSGKYKKVEIKELGELRKEPSL |
| Full length WT Trop2 | 14 | ATGGCGAGAGGTCCCGGGTTGGCAC |
| (DNA) | | CCCCACCGTTGCGGCTTCCTCTTTT |
| | | GCTTCTCGTGCTTGCCGCCGTCACT |
| | | GGGCACACGGCTGCTCAAGATAACT |
| | | GCACTTGCCCCACTAACAAAATGAC |
| | | AGTTTGCAGCCCCGATGGACCTGGT |
| | | GGTCGCTGTCAGTGTAGAGCGCTTG |
| | | GATCTGGAATGGCTGTAGATTGCTC |
| | | CACCCTCACGAGTAAATGTTTGCTG |
| | | TTGAAAGCAAGGATGTCTGCACCTA |
| | | AAAACGCCCGCACACTTGTTCGCCC |
| | | CTCTGAACACGCTCTTGTTGATAAT |
| | | GATGGTCTGTATGATCCCGATTGCG |
| | | ATCCCGAGGGCCGCTTTAAAGCAAG |
| | | GCAATGCAATCAAACGTCCGTTTGC |
| | | TGGTGCGTCAACTCAGTTGGTGTAC |
| | | GGCGCACTGATAAAGGGGACCTGTC |
| | | CCTCCGCTGCGATGAATTGGTTCGC |
| | | ACCCACCACATCCTCATAGATCTCA |
| | | GGCACCGACCAACAGCTGGAGCATT |
| | | CAACCACTCCGACCTCGACGCTGAG |
| | | CTGCGGCGGTTGTTTAGGGAACGGT |
| | | ACCGGCTCCATCCGAAGTTTGTGGC |
| | | CGCAGTTCACTATGAGCAGCCCACT |
| | | ATCCAGATTGAGTTGCGACAGAACA |
| | | CGAGCCAGAAGGCGGCTGGTGACGT |
| | | GGATATAGGCGATGCTGCCTACTAC |
| | | TTCGAACGGGATATAAAGGGAGAGA |
| | | GTCTCTTTCAGGGCCGAGGGGGCCT |
| | | CGATCTGAGGGTAAGAGGCGAACCT |
| | | CTCCAGGTCGAGCGCACGCTGATCT |
| | | ACTACCTGGATGAGATCCCGCCCAA |
| | | ATTTAGCATGAAACGACTTACAGCC |
| | | GGACTCATAGCGGTCATAGTTGTTG |
| | | TTGTCGTGGCCCTGGTCGCAGGTAT |
| | | GGCGGTCCTCGTCATCACGAACAGG |
| | | AGAAAGAGCGGTAAATATAAGAAAG |
| | | TGGAGATTAAGGAGCTTGGGGAACT |
| | | GCGCAAAGAACCCTCATTGTAA |
| Truncated EGFR (tEGFR) | 15 | MLLLVTSLLLCELPHPAFLRKVCNG |
| (polypeptide) | | IGIGEFKDSLSINATNIKHFKNCTS |
| | | ISGDLHILPVAFRGDSFTHTPPLDP |
| | | QELDILKTVKEITGFLLIQAWPENR |
| | | TDLHAFENLEIIRGRTKQHGQFSLA |
| | | VVSLNITSLGLRSLKEISDGDVIIS |
| | | GNKNLCYANTINWKKLFGTSGQKTK |
| | | IISNRGENSCKATGQVCHALCSPEG |
| | | CWGPEPRDCVSCRNVSRGRECVDKS |
| | | QPLSLRPEACRPAAGGAVHTRGLDF |
| | | ACDIYIWAPLAGTCGVLLLSLVITL |
| | | YCWVRGDRYQAYR |
| Truncated EGFR (tEGFR) | 16 | ATGCTGCTGCTGGTGACCAGCCTGC |
| (DNA) | | TGCTGTGCGAGCTGCCACACCCTGC |
| | | CTTCCTGAGGAAAGTGTGTAATGGC |
| | | ATCGGCATCGGCGAGTTTAAGGACA |
| | | GCCTGTCCATCAACGCCACAAATAT |
| | | CAAGCACTTCAAGAACTGTACCTCT |
| | | ATCAGCGGCGACCTGCACATCCTGC |
| | | CAGTGGCCTTCAGAGGCGATTCCTT |
| | | TACACACACCCCACCACTGGACCCA |
| | | CAGGAGCTGGATATCCTGAAGACAG |
| | | TGAAGGAGATCACCGGCTTCCTGCT |
| | | GATCCAGGCATGGCCAGAGAACAGG |
| | | ACAGATCTGCACGCCTTTGAGAATC |
| | | TGGAGATCATCAGAGGCAGGACCAA |
| | | GCAGCACGGCCAGTTCTCTCTGGCC |
| | | GTGGTGAGCCTGAACATCACATCCC |
| | | TGGGCCTGCGCTCTCTGAAGGAGAT |
| | | CAGCGACGGCGATGTGATCATCTCC |
| | | GGCAACAAGAATCTGTGCTATGCCA |
| | | ACACCATCAATTGGAAGAAGCTGTT |
| | | TGGCACATCTGGCCAGAAGACCAAG |
| | | ATCATCAGCAACCGCGGCGAGAATT |
| | | CCTGCAAGGCAACCGGACAGGTGTG |
| | | CCACGCACTGTGTAGCCCTGAGGGA |
| | | TGTTGGGGACCAGAGCCACGCGACT |
| | | GCGTGTCCTGTAGGAACGTGTCTAG |
| | | GGGAAGGGAGTGCGTGGATAAGTCA |
| | | CAGCCTTTGTCACTGCGACCGGAAG |
| | | CATGCAGGCCAGCGGCGGGTGGGGC |
| | | AGTACATACGCGAGGTCTGGACTTT |
| | | GCGTGTGATATTTATATATGGGCAC |
| | | CACTCGCCGGTACGTGCGGGGTTCT |
| | | CTTGCTCTCACTTGTGATAACGCTT |
| | | TATTGTTGGGTACGATAAGGTGACC |
| | | GATATCAAGCTTATCGA |
| Full length WT BCMA | 17 | MLQMAGQCSQNEYFDSLLHACIPCQ |
| (polypeptide) | | LRCSSNTPPLTCQRYCNASVTNSVK |
| | | GTNAILWTCLGLSLLLSLAVFVLMF |
| | | LLRKINSEPLKDEFKNTGSGLLGMA |
| | | NIDLEKSRTGDELLLPRGLEYTVEE |
| | | CTCEDCIKSKPKVDSDHCFPLPAME |
| | | EGATILVTTKTNDYCKSLPAALSAT |
| | | EIEKSISAR |
| Full length WT BCMA | 18 | ATGCTGCAGATGGCTGGCCAGTGCT |
| (DNA) | | CTCAGAACGAGTATTTCGACAGTCT |
| | | ATTGCATGCCTGCATCCCGTGTCAG |
| | | CTGAGGTGTTCTAGCAACACGCCTC |
| | | CCCTGACCTGCCAGCGTTACTGTAA |
| | | TGCTTCCGTCACCAACTCCGTGAAG |
| | | GGCACCAACGCGATCCTTTGGACCT |
| | | GCCTGGGCCTCAGCCTCATCATCTC |
| | | TCTGGCCGTGTTCGTCCTGATGTTT |
| | | TTACTCCGCAAGATTAATTCCGAGC |
| | | CACTGAAGGACGAGTTCAAAAACAC |
| | | TGGCTCCGGTCTGTTGGGAATGGCC |
| | | AACATTGACCTGGAGAAGTCGCGCA |
| | | CCGGTGATGAAATCATCCTGCCGCG |
| | | TGGGTTGGAGTACACCGTGGAGGAG |
| | | TGCACTTGCGAGGACTGTATCAAGT |
| | | CCAAGCCCAAGGTTGATTCAGACCA |
| | | CTGTTTCCCCCTGCCCGCCATGGAA |
| | | GAGGGCGCAACCATCCTGGTGACAA |
| | | CCAAGACTAACGACTACTGTAAAAG |
| | | CCTTCCTGCGGCGCTGAGCGCCACG |
| | | GAGATTGAGAAATCCATCTCGGCTC |
| | | GGTAA |
| BCMA extracellular | 19 | MLQMAGQCSQNEYFDSLLHACIPCQ |
| domain | | LRCSSNTPPLTCQRYCNASVTNSVK |
| | | GTNA |
| CD30 extracellular | 20 | MRVLLAALGLLFLGALRAFPQDRPF |
| domain | | EDTCHGNPSHYYDKAVRRCCYRCPM |
| | | GLFPTQQPQRPTDCRKQCEPDYYLD |
| | | EADRCTACVTCSRDDLVEKTPCAWN |
| | | SSRVCECRPGMFCSTSAVNSCARCF |
| | | FHSVCPAGMIVKFPGTAQKNTVCEP |
| | | ASPGVSPACASPENCKEPSSGTIPQ |
| | | AKPTPVSPATSSASTMPVRGGTRLA |
| | | QEAASKLTRAPDSPSSVGRPSSDPG |
| | | LSPTQPCPEGSGDCRKQCEPDYYLD |
| | | EAGRCTACVSCSRDDLVEKTPCAWN |
| | | SSRTCECRPGMICATSATNSCARCV |
| | | PYPICAAETVTKPQDMAEKDTTFEA |
| | | PPLGTQPDCNPTPENGEAPASTSPT |
| | | QSLLVDSQASKTLPIPTSAPVALSS |
| | | TGKPVLDAG |
| Her2 Domain 4 | 21 | LACHQLCARGHCWGPGPTQCVNCSQ |
| (extracellular) | | FLRGQECVEECRVLQGLPREYVNAR |
| | | HCLPCHPECQPQNGSVTCFGPEADQ |
| | | CVACAHYKDPPFCVARCPSGVKPDL |
| | | SYMPIWKFPDEEGACQPCPINCTHS |
| | | CVDLDDKGCPAEQRASPLT |
| Domain 3 and partial | 22 | RKVCNGIGIGEFKDSLSINATNIKH |
| domain 4 from EGFR | | FKNCTSISGDLHILPVAFRGDSFTH |
| (extracellular) | | TPPLDPQELDILKTVKEITGFLLIQ |
| | | AWPENRTDLHAFENLEIIRGRTKQH |
| | | GQFSLAVVSLNITSLGLRSLKEISD |
| | | GDVIISGNKNLCYANTINWKKLFGT |
| | | SGQKTKIISNRGENSCKATGQVCHA |
| | | LCSPEGCWGPEPRDCVSCRNVSRGR |
| | | ECVDK |
| PDL1 hinge/spacer | 23 | HPPNER |
| CD8 hinge/spacer | 24 | PTIASQPLSLRPEACRPAAGGAVHT |
| | | RGLDFACD |
| PDL1 transmembrane | 25 | THLVILGAILLCLGVALTFIF |
| domain | | |
| CD8 transmembrane | 26 | THLVILGAILLCLGVALTFIF |
| domain | | |
| CD30 transmembrane | 27 | PVLFWVILVLVVVVGSSAFLLCHR |
| domain | | |
| Her2 transmembrane | 28 | SIISAVVGILLVVVLGVVFGILIKR |
| domain | | |
| CD8 intracellular | 29 | RLR |
| residues | | |
| (short) | | |
| CD8 intracellular | 30 | LYCWVR |
| residues | | |
| (long) | | |
| CD317 cytoplasmic | 31 | TSYDYCRVPG |
| domain | | |
| CD3gamma cytoplasmic | 32 | SDKQTLLPG |
| domain | | |
| BCMA cytoplasmic tail | 33 | KINSEPLKDEFKNTGSGLLGMANID |
| | | LEKSRTGDEIILPRGLEYTVEECTC |
| | | EDCIKSKPKVDSDHCFPLPAMEEGA |
| | | TILVTTKTNDYCKSLPAALSATEIE |
| | | KSISAR |
| Tissue-type plasminogen | 34 | MDAMKRGLCCVLLLCGAVFVSPSQE |
| activator signal peptide | | IHARFRRG |
| | | AR |
| CD8α signal peptide | 35 | MALPVTALLLPLALLLHAARP |
| GM-CSFRα signal peptide | 36 | MLLLVTSLLLCELPHPAFL |
| CD8 hinge/spacer (short) | 37 | SQPLSLRPEACRPAAGGAVHTRGLD |
| | | FACD |
| tBCMA [extracellular | 38 | MLQMAGQCSQNEYFDSLLHACIPCQ |
| domain-PDL1 hinge & | | LRCSSNTPPLTCQRYCNASVTNSVK |
| TM plus Trop2 | | GTNAHPPNERTHLVILGAILLCLGV |
| cytoplasmic domain | | ALTFIFRASTNRRKSGKYKKVEIKE |
| (polypeptide)] | | LGELRKEPSL |
| tBCMA [extracellular | 39 | ATGTTGCAAATGGCTGGTCAATGTT |
| domain-PDL1 hinge & | | CACAGAATGAATACTTCGATAGTCT |
| TM plus Trop2 | | GTTGCACGCTTGCATTCCATGTCAA |
| cytoplasmic domain | | CTGAGGTGCTCTAGCAATACACCTC |
| (DNA)] | | CACTCACTTGCCAGAGATACTGCAA |
| | | CGCGTCCGTAACAAACTCAGTCAAG |
| | | GGCACGAATGCGCACCCCCCCAATG |
| | | AAAGAACTCACCTGGTCATTCTCGG |
| | | GGCTATCCTCTTGTGTCTCGGAGTA |
| | | GCGTTGACCTTTATCTTCCGCGCGT |
| | | CCACGAATCGGAGGAAGAGCGGAAA |
| | | GTACAAGAAGGTGGAGATTAAAGAA |
| | | TTGGGGGAACTGCGCAAGGAGCCAA |
| | | GTCTGTAA |
| Trop2 intracellular | 40 | ASTNRRKSGKYKKVEIKELGELRKE |
| region | | PSL |
| BCMA extracellular | 41 | MLQMAGQCSQNEYFDSLLHACIPCQ |
| domain (polypeptide) | | LRCSSNTPPLTCQRYCNASVTNSVK |
| | | GTNA |
| BCMA extracellular | 42 | ATGCTCCAGATGGCTGGCCAGTGTT |
| domain (DNA) | | CGCAGAACGAGTACTTCGACAGTCT |
| | | TCTACACGCCTGTATTCCTTGTCAG |
| | | CTGCGTTGCTCTAGCAACACGCCCC |
| | | CACTGACCTGCCAGCGTTACTGCAA |
| | | TGCTTCCGTCACCAACTCCGTGAAG |
| | | GGCACCAACGCG |

### C. Variant Polypeptides

The following is a discussion of changing the amino acid subunits of a protein to create an equivalent, or even improved, second-generation variant polypeptide or peptide. For example, certain amino acids may be substituted for other amino acids in a protein or polypeptide sequence with or without appreciable loss of interactive binding capacity with structures such as, for example, antigen-binding regions of antibodies or binding sites on substrate molecules. Since it is the interactive capacity and nature of a protein that defines that protein's functional activity, certain amino acid substitutions can be made in a protein sequence and in its corresponding DNA coding sequence, and nevertheless produce a protein with similar or desirable properties. It is thus contemplated by the inventors that various changes may be made in the DNA sequences of genes which encode proteins without appreciable loss of their biological utility or activity.

The term “functionally equivalent codon” is used herein to refer to codons that encode the same amino acid, such as the six different codons for arginine. Also considered are “neutral substitutions” or “neutral mutations” which refers to a change in the codon or codons that encode biologically equivalent amino acids.

Amino acid sequence variants of the disclosure can be substitutional, insertional, or deletion variants. A variation in a polypeptide of the disclosure may affect 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more non-contiguous or contiguous amino acids of the protein or polypeptide, as compared to wild-type. A variant can comprise an amino acid sequence that is at least 50%, 60%, 70%, 80%, or 90%, including all values and ranges there between, identical to any sequence provided or referenced herein. A variant can include 2, 3, 4, 5, 6, 7, 8, 9, 10, 11, 12, 13, 14, 15, 16, 17, 18, 19, 20, or more substitute amino acids.

It also will be understood that amino acid and nucleic acid sequences may include additional residues, such as additional N- or C-terminal amino acids, or 5′ or 3′ sequences, respectively, and yet still be essentially identical as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

Deletion variants typically lack one or more residues of the native or wild type protein. Individual residues can be deleted or a number of contiguous amino acids can be deleted. A stop codon may be introduced (by substitution or insertion) into an encoding nucleic acid sequence to generate a truncated protein.

Insertional mutants typically involve the addition of amino acid residues at a non-terminal point in the polypeptide. This may include the insertion of one or more amino acid residues. Terminal additions may also be generated and can include fusion proteins which are multimers or concatemers of one or more peptides or polypeptides described or referenced herein.

Substitutional variants typically contain the exchange of one amino acid for another at one or more sites within the protein or polypeptide, and may be designed to modulate one or more properties of the polypeptide, with or without the loss of other functions or properties. Substitutions may be conservative, that is, one amino acid is replaced with one of similar chemical properties. “Conservative amino acid substitutions” may involve exchange of a member of one amino acid class with another member of the same class. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Conservative amino acid substitutions may encompass non-naturally occurring amino acid residues, which are typically incorporated by chemical peptide synthesis rather than by synthesis in biological systems. These include peptidomimetics or other reversed or inverted forms of amino acid moieties.

Alternatively, substitutions may be “non-conservative”, such that a function or activity of the polypeptide is affected. Non-conservative changes typically involve substituting an amino acid residue with one that is chemically dissimilar, such as a polar or charged amino acid for a nonpolar or uncharged amino acid, and vice versa. Non-conservative substitutions may involve the exchange of a member of one of the amino acid classes for a member from another class.

### D. Considerations for Substitutions

One skilled in the art can determine suitable variants of polypeptides as set forth herein using well-known techniques. One skilled in the art may identify suitable areas of the molecule that may be changed without destroying activity by targeting regions not believed to be important for activity. The skilled artisan will also be able to identify amino acid residues and portions of the molecules that are conserved among similar proteins or polypeptides. In further embodiments, areas that may be important for biological activity or for structure may be subject to conservative amino acid substitutions without significantly altering the biological activity or without adversely affecting the protein or polypeptide structure.

In making such changes, the hydropathy index of amino acids may be considered. The hydropathy profile of a protein is calculated by assigning each amino acid a numerical value (“hydropathy index”) and then repetitively averaging these values along the peptide chain. Each amino acid has been assigned a value based on its hydrophobicity and charge characteristics. They are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (1.6); histidine (−3.2); glutamate (−3.5); glutamine (−3.5); aspartate (−3.5); asparagine (−3.5); lysine (−3.9); and arginine (−4.5). The importance of the hydropathy amino acid index in conferring interactive biologic function on a protein is generally understood in the art (Kyte et al., J. Mol. Biol. 157:105-131 (1982)). It is accepted that the relative hydropathic character of the amino acid contributes to the secondary structure of the resultant protein or polypeptide, which in turn defines the interaction of the protein or polypeptide with other molecules, for example, enzymes, substrates, receptors, DNA, antibodies, antigens, and others. It is also known that certain amino acids may be substituted for other amino acids having a similar hydropathy index or score, and still retain a similar biological activity. In making changes based upon the hydropathy index, in certain embodiments, the substitution of amino acids whose hydropathy indices are within ±2 is included. In some aspects of the present disclosure, those that are within ±1 are included, and in other aspects of the present disclosure, those within ±0.5 are included.

It also is understood in the art that the substitution of like amino acids can be effectively made based on hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. In certain embodiments, the greatest local average hydrophilicity of a protein, as governed by the hydrophilicity of its adjacent amino acids, correlates with its immunogenicity and antigen binding, that is, as a biological property of the protein. The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). In making changes based upon similar hydrophilicity values, in certain embodiments, the substitution of amino acids whose hydrophilicity values are within ±2 are included, in other embodiments, those which are within ±1 are included, and in still other embodiments, those within ±0.5 are included. In some instances, one may also identify epitopes from primary amino acid sequences based on hydrophilicity. These regions are also referred to as “epitopic core regions.” It is understood that an amino acid can be substituted for another having a similar hydrophilicity value and still produce a biologically equivalent and immunologically equivalent protein.

Additionally, one skilled in the art can review structure-function studies identifying residues in similar polypeptides or proteins that are important for activity or structure. In view of such a comparison, one can predict the importance of amino acid residues in a protein that correspond to amino acid residues important for activity or structure in similar proteins. One skilled in the art may opt for chemically similar amino acid substitutions for such predicted important amino acid residues.

One skilled in the art can also analyze the three-dimensional structure and amino acid sequence in relation to that structure in similar proteins or polypeptides. In view of such information, one skilled in the art may predict the alignment of amino acid residues of an antibody with respect to its three-dimensional structure. One skilled in the art may choose not to make changes to amino acid residues predicted to be on the surface of the protein, since such residues may be involved in important interactions with other molecules. Moreover, one skilled in the art may generate test variants containing a single amino acid substitution at each desired amino acid residue. These variants can then be screened using standard assays for binding and/or activity, thus yielding information gathered from such routine experiments, which may allow one skilled in the art to determine the amino acid positions where further substitutions should be avoided either alone or in combination with other mutations. Various tools available to determine secondary structure can be found on the world wide web, for example at expasy.org/proteomics/protein_structure.

In some embodiments of the disclosure, amino acid substitutions are made that: (1) reduce susceptibility to proteolysis, (2) reduce susceptibility to oxidation, (3) alter binding affinity for forming protein complexes, (4) alter ligand or antigen binding affinities, and/or (5) confer or modify other physicochemical or functional properties on such polypeptides. For example, single or multiple amino acid substitutions (in certain embodiments, conservative amino acid substitutions) may be made in the naturally occurring sequence. Substitutions can be made in that portion of the antibody that lies outside the domain(s) forming intermolecular contacts. In such embodiments, conservative amino acid substitutions can be used that do not substantially change the structural characteristics of the protein or polypeptide (e.g., one or more replacement amino acids that do not disrupt the secondary structure that characterizes the native antibody).

### E. B-Cell Maturation Antigen (BCMA)

B-cell Maturation Antigen (BCMA), also known as tumor necrosis factor receptor superfamily member 17 or TNFRSF17, is preferentially expressed on plasma cells and is highly expressed in a variety of hematologic malignancies including multiple myeloma. BCMA is encoded by the TNFRSF17 gene. An example mRNA is characterized by RefSeq accession number NM_001192. An example protein is characterized by RefSeq accession number NP_001183. The complete polypeptide sequence of human BCMA is provided as SEQ ID NO:17. The complete DNA sequence of the BCMA gene (TNFRSF17) is provided as SEQ ID NO:18.

### F. Programmed Cell Death 1 Ligand 1 (PDL1)

Programmed cell death 1 ligand 1 (PDL1), also known as CD274 or B7-H1, is an immune inhibitory receptor ligand expressed by hematopoietic cells, immune cells, and various types of tumor cells. An example mRNA is characterized by RefSeq accession numbers NM_001267706 and NM_014143. An example protein is characterized by RefSeq accession numbers NP_001254635 and NP_054862.

### G. CD30

CD30, also known as Tumor necrosis factor receptor superfamily member 8 or TNFRSF8, is encoded by the TNFRSF8 gene. An example mRNA is characterized by RefSeq accession number NM_001243. An example protein is characterized by RefSeq accession number NP_001234.

### H. Her2

Her2 (or “HER2”), also known as receptor tyrosine-protein kinase erbB-2 (erbB-2 or ErbB2), Neu, or Her2/neu, is a member of the epidermal growth factor (EGF) receptor family of receptor tyrosine kinases, and amplification and/or overexpression of Her2 has been reported in numerous cancers, including breast and ovarian tumors. The extracellular domain of HER2 comprises four domains, Domain I (amino acid residues from about 1-195), Domain II (amino acid residues from about 196-319), Domain III (amino acid residues from about 320-488), and Domain IV (amino acid residues from about 489-630) (residue numbering without signal peptide). See Garrett et al., Mol. Cell. 11: 495-505 (2003), Cho et al., Nature 421: 756-760 (2003), Franklin et al., Cancer Cell 5:317-328 (2004), Plowman et al., Proc. Natl. Acad. Sci. 90:1746-1750 (1993), and U.S. Pat. No. 8,652,474, each incorporated herein by reference. Her2 is encoded by the ERBB2 gene. An example mRNA is characterized by RefSeq accession number NM_001005862. An example protein is characterized by RefSeq accession number NP_001005862.

### I. Trop2

Trop2 (or “Trop-2”), also known as tumor-associated calcium signal transducer 2, is a carcinoma-associated antigen. Trop2 is encoded by the TACSTD2 gene. An example mRNA is characterized by RefSeq accession number NM_002353. An example protein is characterized by RefSeq accession number NP_002344. The complete polypeptide sequence of human Trop2 is provided as SEQ ID NO:13. The complete DNA sequence of the Trop2 gene (TACSTD2) is provided as SEQ ID NO:14.

### J. EGFR

Epidermal growth factor receptor (EGFR), also known as erbB-1, is preferentially expressed on plasma cells and is highly expressed in a variety of hematologic malignancies including multiple myeloma. The extracellular domain of EGFR is composed of 4 domains (from the N-terminus, referred to as domain I, domain II, domain III, and domain IV, or also referred to as L1, S1, L2, and S2 domains, respectively) (see Bajaj, M. et. al. Biochim. Biophys. Acta 916, 220-226 (1987) and U.S. Pat. No. 7,514,240, each incorporated herein by reference). EGFR is encoded by the EGFR gene. An example mRNA is characterized by RefSeq accession number NM_005228. An example protein is characterized by RefSeq accession number NP_005219.

### II. Nucleic Acids

In certain embodiments, nucleic acid sequences can exist in a variety of instances such as: isolated segments and recombinant vectors of incorporated sequences or recombinant polynucleotides encoding one or both chains of an antibody, or a fragment, derivative, mutein, or variant thereof, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, and complementary sequences of the foregoing described herein. Nucleic acids that encode the epitope to which certain of the antibodies provided herein are also provided. Nucleic acids encoding fusion proteins that include these peptides are also provided. The nucleic acids can be single-stranded or double-stranded and can comprise RNA and/or DNA nucleotides and artificial variants thereof (e.g., peptide nucleic acids).

The term “polynucleotide” refers to a nucleic acid molecule that either is recombinant or has been isolated from total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar protein.

In certain embodiments, there are polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence provided herein using the methods described herein (e.g., BLAST analysis using standard parameters). In certain aspects, the isolated polynucleotide will comprise a nucleotide sequence encoding a polypeptide that has at least 90%, preferably 95% and above, identity to an amino acid sequence described herein, over the entire length of the sequence; or a nucleotide sequence complementary to said isolated polynucleotide.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. The nucleic acids can be any length. They can be, for example, 5, 10, 15, 20, 25, 30, 35, 40, 45, 50, 75, 100, 125, 175, 200, 250, 300, 350, 400, 450, 500, 750, 1000, 1500, 3000, 5000 or more nucleotides in length, and/or can comprise one or more additional sequences, for example, regulatory sequences, and/or be a part of a larger nucleic acid, for example, a vector. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

### A. Hybridization

The nucleic acids that hybridize to other nucleic acids under particular hybridization conditions. Methods for hybridizing nucleic acids are well known in the art. See, e.g., Current Protocols in Molecular Biology, John Wiley and Sons, N.Y. (1989), 6.3.1-6.3.6. As defined herein, a moderately stringent hybridization condition uses a prewashing solution containing 5× sodium chloride/sodium citrate (SSC), 0.5% SDS, 1.0 mM EDTA (pH 8.0), hybridization buffer of about 50% formamide, 6×SSC, and a hybridization temperature of 55° C. (or other similar hybridization solutions, such as one containing about 50% formamide, with a hybridization temperature of 42° C.), and washing conditions of 60° C. in 0.5×SSC, 0.1% SDS. A stringent hybridization condition hybridizes in 6×SSC at 45° C., followed by one or more washes in 0.1×SSC, 0.2% SDS at 68° C. Furthermore, one of skill in the art can manipulate the hybridization and/or washing conditions to increase or decrease the stringency of hybridization such that nucleic acids comprising nucleotide sequence that are at least 65%, at least 70%, at least 75%, at least 80%, at least 85%, at least 90%, at least 95%, at least 96%, at least 97%, at least 98% or at least 99% identical to each other typically remain hybridized to each other.

The parameters affecting the choice of hybridization conditions and guidance for devising suitable conditions are set forth by, for example, Sambrook, Fritsch, and Maniatis (Molecular Cloning: A Laboratory Manual, Cold Spring Harbor Laboratory Press, Cold Spring Harbor, N.Y., chapters 9 and 11 (1989); Current Protocols in Molecular Biology, Ausubel et al., eds., John Wiley and Sons, Inc., sections 2.10 and 6.3-6.4 (1995), both of which are herein incorporated by reference in their entirety for all purposes) and can be readily determined by those having ordinary skill in the art based on, for example, the length and/or base composition of the DNA.

### B. Mutation

Changes can be introduced by mutation into a nucleic acid, thereby leading to changes in the amino acid sequence of a polypeptide (e.g., an antibody or antibody derivative) that it encodes. Mutations can be introduced using any technique known in the art. In one embodiment, one or more particular amino acid residues are changed using, for example, a site-directed mutagenesis protocol. In another embodiment, one or more randomly selected residues are changed using, for example, a random mutagenesis protocol. However it is made, a mutant polypeptide can be expressed and screened for a desired property.

Mutations can be introduced into a nucleic acid without significantly altering the biological activity of a polypeptide that it encodes. For example, one can make nucleotide substitutions leading to amino acid substitutions at non-essential amino acid residues. Alternatively, one or more mutations can be introduced into a nucleic acid that selectively changes the biological activity of a polypeptide that it encodes. See, e.g., Romain Studer et al., Biochem. J. 449:581-594 (2013). For example, the mutation can quantitatively or qualitatively change the biological activity. Examples of quantitative changes include increasing, reducing or eliminating the activity. Examples of qualitative changes include altering the antigen specificity of an antibody.

### III. Infinite Immune Cells

Certain embodiments of the present disclosure concern immune cells that are engineered to express one or more genes. The expression of the one or more genes directly or indirectly results in the increased lifespan of the cells compared to cells that lack the expression of the one or more genes. In particular embodiments, the cells are manipulated to express the one or more genes, including one or more heterologous genes. In other cases, the cells are manipulated to have upregulation of expression of the one or more genes that are endogenous to the cells, such as through manipulation of one or more regulatory elements of the one or more endogenous genes to the cells.

In particular embodiments, immune cells are manipulated to express BCL6 and one or more pro-survival genes or anti-apoptotic genes or cell survival-promoting genes (and there may or may not be overlap in a gene that is classified as pro-survival or anti-apoptotic or cell survival-promoting). As used herein, the pro-survival gene refers to a nucleic acid polymer that can exert anti-apoptosis function or promote survival by any mechanism. The nucleic acid polymer that can exert anti-apoptosis function may be one or more of Bcl2 family genes such as BCL-xL, BCL-2, MCL-1, Bcl-w, Bfl-1, BCL-B, etc. The nucleic acid polymer that can exert anti-apoptosis function may be one or more of inhibitor of apoptosis (IAP) family genes, such as XIAP, c-IAP1, C-IAP2, NAIP, and Survivin, etc. The nucleic acid polymer that can exert anti-apoptosis function may be able to inhibit or knock out expression of one or more caspases that play a role in apoptosis, such as Caspase-1, Caspase-2, Caspase-3, Caspase-4, Caspase-5, Caspase-6, Caspase-7, Caspase-8, Caspase-9, Caspase-10, Caspase-11, Caspase-12, Caspase-13, Caspase-14. Nucleic acid polymers for knockdown or knock-out could be an shRNA expression cassette, or these caspase genes can also be knocked out by gene editing method (CRISPR, TALEN, Zinc finger method, etc.). The nucleic acid polymer that can exert anti-apoptosis function may be able to inhibit or knock out expression of one or more pro-apoptotic genes, such as BIM, Puma, Noxa, Bik, Bmf, Bad, Hrk, Bid, BAX, BAK, BOK, etc. The nucleic acid polymer that can exert anti-apoptosis function may have an anti-apoptotic effect, such as insulin-like growth factor (IGF-1), Hsp70, Hsp27, cFLIP, BNIP3, FADD, Akt, and NF-1B, Raf-1 and MEK1, p90Rsk, C-Jun, BNIP2, BAG1, HSPA9, HSP90B1,miRNA21, miR-106b-25, miR-206, miR-221/222, miR-17-92, miR-133, miR-143, miR-145, miR-155, miR-330, etc.

Infinite T cells may be generated with either wild type or mutant BCL6. The inventors determined that infinite T cells could be generated with either wildtype BCL6 or mutant BCL6 with a single particular nucleotide difference—the codon of the amino acid at position 395 in wild type BCL6 is CCT (encoding Proline/P) and the codon of the amino acid at position 395 in mutant BCL6 is CTT (encoding Leucine/L). The nucleotide and amino acid sequences for the two BCL6 genes are shown below (with the point of mutation in the wildtype sequence being underlined).

The aa sequence of wildtype BCL6:
(SEQ ID NO: 43)
MASPADSCIQFTRHASDVLLNLNRLRSRDILTDVVIVVSREQFRA
HKTVLMACSGLFYSIFTDQLKCNLSVINLDPEINPEGFCILLDFM
YTSRLNLREGNIMAVMATAMYLQMEHVVDTCRKFIKASEAEMVSA
IKPPREEFLNSRMLMPQDIMAYRGREVVENNLPLRSAPGCESRAF
APSLYSGLSTPPASYSMYSHLPVSSLLFSDEEFRDVRMPVANPFP
KERALPCDSARPVPGEYSRPTLEVSPNVCHSNIYSPKETIPEEAR
SDMHYSVAEGLKPAAPSARNAPYFPCDKASKEEERPSSEDEIALH
FEPPNAPLNRKGLVSPQSPQKSDCQPNSPTESCSSKNACILQASG
SPPAKSPTDPKACNWKKYKFIVLNSLNQNAKPEGPEQAELGRLSP
RAYTAPPACQPPMEPENLDLQSPTKLSASGEDSTIPQASRLNNIV
NRSMTGSPRSSSESHSPLYMHPPKCTSCGSQSPQHAEMCLHTAGP
TFPEEMGETQSEYSDSSCENGAFFCNECDCRFSEEASLKRHTLQT
HSDKPYKCDRCQASFRYKGNLASHKTVHTGEKPYRCNICGAQFNR
PANLKTHTRIHSGEKPYKCETCGARFVQVAHLRAHVLIHTGEKPY
PCEICGTRFRHLQTLKSHLRIHTGEKPYHCEKCNLHFRHKSQLRL
HLRQKHGAITNTKVQYRVSATDLPPELPKAC
The nucleotide sequence of wildtype BCL6
(with the codon for the point of mutation
in the wildtype sequence being underlined):
(SEQ ID NO: 44)
ATGgcctcgccggctgacagctgtatccagttcacccgccatgcc
agtgatgttcttctcaaccttaatcgtctccggagtcgagacatc
ttgactgatgttgtcattgttgtgagccgtgagcagtttagagcc
cataaaacggtcctcatggcctgcagtggcctgttctatagcatc
tttacagaccagttgaaatgcaaccttagtgtgatcaatctagat
cctgagatcaaccctgagggattctgcatcctcctggacttcatg
tacacatctcggctcaatttgcgggagggcaacatcatggctgtg
atggccacggctatgtacctgcagatggagcatgttgtggacact
tgccggaagtttattaaggccagtgaagcagagatggtttctgcc
atcaagcctcctcgtgaagagttcctcaacagccggatgctgatg
ccccaagacatcatggcctatcggggtcgtgaggtggtggagaac
aacctgccactgaggagcgcccctgggtgtgagagcagagccttt
gcccccagcctgtacagtggcctgtccacacegccagcctcttat
tccatgtacagccacctccctgtcagcagcctcctcttctccgat
gaggagtttcgggatgtccggatgcctgtggccaaccccttcccc
aaggagcgggcactcccatgtgatagtgccaggccagtccctggt
gagtacagccggccgactttggaggtgtcccccaatgtgtgccac
agcaatatctattcacccaaggaaacaatcccagaagaggcacga
agtgatatgcactacagtgtggctgagggcctcaaacctgctgcc
ccctcagcccgaaatgccccctacttcccttgtgacaaggccagc
aaagaagaagagagaccctcctcggaagatgagattgccctgcat
ttcgagccccccaatgcacccctgaaccggaagggtctggttagt
ccacagagcccccagaaatctgactgccagcccaactcgcccaca
gagtcctgcagcagtaagaatgcctgcatcctccaggcttctggc
tcccctccagccaagagccccactgaccccaaagcctgcaactgg
aagaaatacaagttcatcgtgctcaacagcctcaaccagaatgcc
aaaccagaggggcCtgagcaggctgagctgggccgcctttcccca
cgagcctacacggccccacctgcctgccagccacccatggagcct
gagaaccttgacctccagtccccaaccaagctgagtgccagcggg
gaggactccaccatcccacaagccagccggctcaataacatcgtt
aacaggtccatgacgggctctccccgcagcagcagegagagccac
tcaccactctacatgcaccccccgaagtgcacgtcctgeggctct
cagtccccacagcatgcagagatgtgcctccacaccgctggcccc
acgttccctgaggagatgggagagacccagtctgagtactcagat
tctagctgtgagaacggggccttcttctgcaatgagtgtgactgc
cgcttctctgaggaggcctcactcaagaggcacacgctgcagacc
cacagtgacaaaccctacaagtgtgaccgctgccaggcctccttc
cgctacaagggcaacctcgccagccacaagaccgtccataccggt
gagaaaccctatcgttgcaacatctgtggggcccagttcaaccgg
ccagccaacctgaaaacccacactegaattcactctggagagaag
ccctacaaatgcgaaacctgcggagccagatttgtacaggtggcc
cacctccgtgcccatgtgcttatccacactggtgagaagccctat
ccctgtgaaatctgtggcacccgtttccggcaccttcagactctg
aagagccacctgcgaatccacacaggagagaaaccttaccattgt
gagaagtgtaacctgcatttccgtcacaaaagccagctgcgactt
cacttgcgccagaagcatggcgccatcaccaacaccaaggtgcaa
taccgcgtgtcagccactgacctgcctccggagctccccaaagcc
tgc
The aa sequence of mutant BCL6 (the leucine
mutation is underlined):
(SEQ ID NO: 45)
MASPADSCIQFTRHASDVLLNLNRLRSRDILTDVVIVVSREQFRA
HKTVLMACSGLFYSIFTDQLKCNLSVINLDPEINPEGFCILLDFM
YTSRLNLREGNIMAVMATAMYLQMEHVVDTCRKFIKASEAEMVSA
IKPPREEFLNSRMLMPQDIMAYRGREVVENNLPLRSAPGCESRAF
APSLYSGLSTPPASYSMYSHLPVSSLLFSDEEFRDVRMPVANPFP
KERALPCDSARPVPGEYSRPTLEVSPNVCHSNIYSPKETIPEEAR
SDMHYSVAEGLKPAAPSARNAPYFPCDKASKEEERPSSEDEIALH
FEPPNAPLNRKGLVSPQSPQKSDCQPNSPTESCSSKNACILQASG
SPPAKSPTDPKACNWKKYKFIVLNSLNQNAKPEGLEQAELGRLSP
RAYTAPPACQPPMEPENLDLQSPTKLSASGEDSTIPQASRLNNIV
NRSMTGSPRSSSESHSPLYMHPPKCTSCGSQSPQHAEMCLHTAGP
TFPEEMGETQSEYSDSSCENGAFFCNECDCRFSEEASLKRHTLQT
HSDKPYKCDRCQASFRYKGNLASHKTVHTGEKPYRCNICGAQFNR
PANLKTHTRIHSGEKPYKCETCGARFVQVAHLRAHVLIHTGEKPY
PCEICGTRFRHLQTLKSHLRIHTGEKPYHCEKCNLHFRHKSQLRL
HLRQKHGAITNTKVQYRVSATDLPPELPKAC
The nucleotide sequence of mutant BCL6
(the codon for leucine is underlined):
(SEQ ID NO: 46)
ATGgcctcgccggctgacagctgtatccagttcacccgccatgcc
agtgatgttcttctcaaccttaatcgtctccggagtcgagacatc
ttgactgatgttgtcattgttgtgagccgtgagcagtttagagcc
cataaaacggtcctcatggcctgcagtggcctgttctatagcatc
tttacagaccagttgaaatgcaaccttagtgtgatcaatctagat
cctgagatcaaccctgagggattctgcatcctcctggacttcatg
tacacatctcggctcaatttgcgggagggcaacatcatggctgtg
atggccacggctatgtacctgcagatggagcatgttgtggacact
tgccggaagtttattaaggccagtgaagcagagatggtttctgcc
atcaagcctcctcgtgaagagttcctcaacagccggatgctgatg
ccccaagacatcatggcctatcggggtcgtgaggtggtggagaac
aacctgccactgaggagcgcccctgggtgtgagagcagagccttt
gcccccagcctgtacagtggcctgtccacacegccagcctcttat
tccatgtacagccacctccctgtcagcagcctcctcttctccgat
gaggagtttcgggatgtccggatgcctgtggccaaccccttcccc
aaggagcgggcactcccatgtgatagtgccaggccagtccctggt
gagtacagccggccgactttggaggtgtcccccaatgtgtgccac
agcaatatctattcacccaaggaaacaatcccagaagaggcacga
agtgatatgcactacagtgtggctgagggcctcaaacctgctgcc
ccctcagcccgaaatgccccctacttcccttgtgacaaggccagc
aaagaagaagagagaccctcctcggaagatgagattgccctgcat
ttcgagccccccaatgcacccctgaaccggaagggtctggttagt
ccacagagcccccagaaatctgactgccagcccaactcgcccaca
gagtcctgcagcagtaagaatgcctgcatcctccaggcttctggc
tcccctccagccaagagccccactgaccccaaagcctgcaactgg
aagaaatacaagttcatcgtgctcaacagcctcaaccagaatgcc
aaaccagaggggcTtgagcaggctgagctgggccgcctttcccca
cgagcctacacggccccacctgcctgccagccacccatggagcct
gagaaccttgacctccagtccccaaccaagctgagtgccagcggg
gaggactccaccatcccacaagccagccggctcaataacatcgtt
aacaggtccatgacgggctctccccgcagcagcagcgagagccac
tcaccactctacatgcaccccccgaagtgcacgtcctgcggctct
cagtccccacagcatgcagagatgtgcctccacacegctggcccc
acgttccctgaggagatgggagagacccagtctgagtactcagat
tctagctgtgagaacggggccttcttctgcaatgagtgtgactgc
cgcttctctgaggaggcctcactcaagaggcacacgctgcagacc
cacagtgacaaaccctacaagtgtgaccgctgccaggcctccttc
cgctacaagggcaacctcgccagccacaagaccgtccataccggt
gagaaaccctatcgttgcaacatctgtggggcccagttcaaccgg
ccagccaacctgaaaacccacactcgaattcactctggagagaag
ccctacaaatgcgaaacctgcggagccagatttgtacaggtggcc
cacctccgtgcccatgtgcttatccacactggtgagaagccctat
ccctgtgaaatctgtggcacccgtttccggcaccttcagactctg
aagagccacctgcgaatccacacaggagagaaaccttaccattgt
gagaagtgtaacctgcatttccgtcacaaaagccagctgcgactt
cacttgcgccagaagcatggcgccatcaccaacaccaaggtgcaa
taccgcgtgtcagccactgacctgcctccggagctccccaaagcc
tgc

The immune cells may be any kind of immune cells, including T cells (e.g., regulatory T cells, CD4⁺ T cells, CD8⁺ T cells, alpha beta T cells, gamma-delta T cells, or a mixture thereof), NK cells, invariant NKT cells, NKT cells, innate lymphoid cells, or a mixture thereof. The immune cells may be virus-specific, express a CAR, and/or express a TCR. In some embodiments, the cells are monocytes or granulocytes, e.g., myeloid cells, macrophages, neutrophils, dendritic cells (DCs), mast cells, eosinophils, and/or basophils. Also provided herein are methods of producing and engineering the immune cells as well as methods of using and administering the cells for adoptive cell therapy, in which case the cells may be autologous or allogeneic. Thus, the immune cells may be used as immunotherapy, such as to target cancer cells. These immune cells may be used for therapy as a single cell type or as a combination of multiple immune cell types. In specific embodiments, the immune cells are CD3+, CD4+, CD8+, CD16+, or a mixture thereof.

The immune cells may be isolated from subjects, particularly human subjects. The immune cells can be obtained from a subject of interest, such as a subject suspected of having a particular disease or condition, a subject suspected of having a predisposition to a particular disease or condition, or a subject who is undergoing therapy for a particular disease or condition. Immune cells can be collected from any location in which they reside in the subject including, but not limited to, blood, cord blood, spleen, thymus, lymph nodes, and bone marrow. The isolated immune cells may be used directly, or they can be stored for a period of time, such as by freezing.

The immune cells may be enriched/purified from any tissue where they reside including, but not limited to, blood (including blood collected by blood banks or cord blood banks), spleen, bone marrow, tissues removed and/or exposed during surgical procedures, and tissues obtained via biopsy procedures. Tissues/organs from which the immune cells are enriched, isolated, and/or purified may be isolated from both living and non-living subjects, wherein the non-living subjects are organ donors. In particular embodiments, the immune cells are isolated from blood, such as peripheral blood or cord blood. In some aspects, immune cells isolated from cord blood have enhanced immunomodulation capacity, such as measured by CD4- or CD8-positive T cell suppression. In specific aspects, the immune cells are isolated from pooled blood, particularly pooled cord blood, for enhanced immunomodulation capacity. The pooled blood may be from 2 or more sources, such as 3, 4, 5, 6, 7, 8, 9, 10 or more sources (e.g., donor subjects).

The population of immune cells can be obtained from a subject in need of therapy or suffering from a disease associated with reduced immune cell activity. Thus, the cells will be autologous to the subject in need of therapy. Alternatively, the population of immune cells can be obtained from a donor, such as a partially or fully histocompatibility matched donor or fully histocompatibility mismatched donor. The immune cell population can be harvested from the peripheral blood, cord blood, bone marrow, spleen, or any other organ/tissue in which immune cells reside in said subject or donor. The immune cells can be isolated from a pool of subjects and/or donors, such as from pooled cord blood.

When the population of immune cells is obtained from a donor distinct from the subject, the donor may be allogeneic, provided the cells obtained are subject-compatible in that they can be introduced into the subject. Allogeneic donor cells are may or may not be human-leukocyte-antigen (HLA)-compatible.

Additional methods and compositions related to infinite immune cells are described in PCT Patent Application Publication No. WO/2021/034982, incorporated herein by reference in its entirety.

### A. T Cells

In some embodiments, the immune cells are T cells. Several basic approaches for the derivation, activation and expansion of functional anti-tumor effector cells have been described in the last two decades. These include: autologous cells, such as tumor-infiltrating lymphocytes (TILs); T cells activated ex-vivo using autologous DCs or PBMCs, lymphocytes, artificial antigen-presenting cells (APCs) or beads coated with T cell ligands and activating antibodies, or cells isolated by virtue of capturing target cell membrane; allogeneic cells naturally expressing anti-host tumor T cell receptor (TCR); and non-tumor-specific autologous or allogeneic cells genetically reprogrammed or “redirected” to express tumor-reactive TCR or chimeric TCR molecules displaying antibody-like tumor recognition capacity known as “T-bodies”. These approaches have given rise to numerous protocols for T cell preparation and immunization which can be used in the methods described herein.

In some embodiments, the T cells are derived from the blood, bone marrow, lymph, umbilical cord, or lymphoid organs. In some aspects, the cells are human cells. The cells typically are primary cells, such as those isolated directly from a subject and/or isolated from a subject and frozen. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, as described herein, and re-introducing them into the same patient, before or after cryopreservation.

Among the sub-types and subpopulations of T cells (e.g., CD4⁺ and/or CD8⁺ T cells) are naive T (T_{N}) cells, effector T cells (T_{EFF}), memory T cells and sub-types thereof, such as stem cell memory T (TSC_{M}), central memory T (TC_{M}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and gamma/delta T cells.

In some embodiments, one or more of the T cell populations is enriched for or depleted of cells that are positive for a specific marker, such as surface markers, or that are negative for a specific marker. In some cases, such markers are those that are absent or expressed at relatively low levels on certain populations of T cells (e.g., non-memory cells) but are present or expressed at relatively higher levels on certain other populations of T cells (e.g., memory cells).

In some embodiments, T cells are separated from a PBMC sample by negative selection of markers expressed on non-T cells, such as B cells, monocytes, or other white blood cells, such as CD14. In some aspects, a CD4+ or CD8+ selection step is used to separate CD4+ helper and CD8+ cytotoxic T cells. Such CD4+ and CD8+ populations can be further sorted into sub-populations by positive or negative selection for markers expressed or expressed to a relatively higher degree on one or more naive, memory, and/or effector T cell subpopulations.

In some embodiments, CD8⁺ T cells are further enriched for or depleted of naive, central memory, effector memory, and/or central memory stem cells, such as by positive or negative selection based on surface antigens associated with the respective subpopulation. In some embodiments, enrichment for central memory T (TC_{M}) cells or stem cell memory cells is carried out to increase efficacy, such as to improve long-term survival, expansion, and/or engraftment following administration, which in some aspects is particularly robust in such sub-populations.

In some embodiments, the T cells are autologous T cells. In this method, tumor samples are obtained from patients and a single cell suspension is obtained. The single cell suspension can be obtained in any suitable manner, e.g., mechanically (disaggregating the tumor using, e.g., a GENTLEMACS™ Dissociator, Miltenyi Biotec, Auburn, Calif.) or enzymatically (e.g., collagenase or DNase). Single-cell suspensions of tumor enzymatic digests are cultured in interleukin-2 (IL-2) or other growth factors.

The cultured T cells can be pooled and rapidly expanded. Rapid expansion provides an increase in the number of antigen-specific T-cells of at least about 50-fold (e.g., 50-, 60-, 70-, 80-, 90-, or 100-fold, or greater) over a period of about 10 to about 14 days. More preferably, rapid expansion provides an increase of at least about 200-fold (e.g., 200-, 300-, 400-, 500-, 600-, 700-, 800-, 900-, or greater) over a period of about 10 to about 14 days.

Expansion can be accomplished by any of a number of methods as are known in the art. For example, T cells can be rapidly expanded using non-specific T-cell receptor stimulation in the presence of feeder lymphocytes and either interleukin-2 (IL-2) or interleukin-15 (IL-15), with IL-2 being preferred. The non-specific T-cell receptor stimulus can include around 30 ng/ml of OKT3, a mouse monoclonal anti-CD3 antibody (available from Ortho-McNeil®, Raritan, N.J.). Alternatively, T cells can be rapidly expanded by stimulation of peripheral blood mononuclear cells (PBMC) in vitro with one or more antigens (including antigenic portions thereof, such as epitope(s), or a cell) of the cancer, which can be optionally expressed from a vector, such as an human leukocyte antigen A2 (HLA-A2) binding peptide or peptides binding to other MHC class I or class II molecules, in the presence of a T-cell growth factor, such as 300 IU/ml IL-2 or IL-15, with IL-2 being preferred. The in vitro-induced T-cells are rapidly expanded by re-stimulation with the same antigen(s) of the cancer pulsed onto HLA-A2-expressing antigen-presenting cells or antigen-presenting cells expressing other HLA molecules. The in vitro-induced T-cells may also be expanded in the absence of antigen-presenting cells.

The autologous T cells can be modified to express a T cell growth or differentiation factor that promotes the growth, differentiation, and activation of the autologous T cells. Suitable T cell growth factors include, for example, interleukin (IL)-2, IL-7, IL-15, IL-18, IL-21, and IL-12. Suitable methods of modification are known in the art. See, for instance, Sambrook et al., Molecular Cloning: A Laboratory Manual, 3^{rd} ed., Cold Spring Harbor Press, Cold Spring Harbor, N.Y. 2001; and Ausubel et al., Current Protocols in Molecular Biology, Greene Publishing Associates and John Wiley & Sons, N Y, 1994. In particular aspects, modified autologous T cells express the T cell growth factor at high levels. T cell growth factor coding sequences, such as that of IL-12, are readily available in the art, as are promoters, the operable linkage of which to a T cell growth factor coding sequence promote high-level expression.

### B. NK Cells

In some embodiments, the immune cells are natural killer (NK) cells. NK cells are a subpopulation of lymphocytes that have spontaneous cytotoxicity against a variety of tumor cells, virus-infected cells, and some normal cells in the bone marrow and thymus. NK cells differentiate and mature in the bone marrow, lymph nodes, spleen, tonsils, and thymus. NK cells can be detected by specific surface markers, such as CD16, CD56, and/or CD8 in humans. NK cells do not express T cell antigen receptors, the pan T marker CD3, or surface immunoglobulin B cell receptors.

In certain embodiments, NK cells are derived from human peripheral blood mononuclear cells (PBMC), unstimulated leukapheresis products (PBSC), human embryonic stem cells (hESCs), induced pluripotent stem cells (iPSCs), bone marrow, tissues, or umbilical cord blood by methods well known in the art.

### C. NKT Cells

Natural killer T (NKT) cells are a heterogeneous group of T cells that share properties of both T cells and natural killer cells. Many of these cells recognize the non-polymorphic CD1d molecule, an antigen-presenting molecule that binds self and foreign lipids and glycolipids. They constitute only approximately 0.1% of all peripheral blood T cells. NKT cells are a subset of T cells that co-express an αβ T-cell receptor, but also express a variety of molecular markers that are typically associated with NK cells, such as NK1.1. Invariant natural killer T (iNKT) cells express high levels of and are dependent on the transcriptional regulator promyelocytic leukemia zinc finger for their development. Currently, there are five major distinct iNKT cell subsets. These subset cells produce a different set of cytokines once activated. The subtypes iNKT1, iNKT2 and iNKT17 mirror Th cell subsets in cytokine production. In addition, there are subtypes specialized in T follicular helper-like function and IL-10 dependent regulatory functions.

### D. Innate Lymphoid Cells

Innate lymphoid cells (ILCs) are a group of innate immune cells that are derived from common lymphoid progenitor (CLP) and belong to the lymphoid lineage. These cells are defined by absence of antigen specific B or T cell receptor because of the lack of recombination activating gene (RAG). ILCs do not express myeloid or dendritic cell markers. They play a role in protective immunity and the regulation of homeostasis and inflammation, so their dysregulation can lead to immune pathology such as allergy, bronchial asthma and autoimmune disease. ILCs can be divided based on the cytokines that they can produce, and the transcription factors that regulate their development and function.

### IV. Formulations and Culture of the Cells

In particular embodiments, cells of the disclosure may be specifically formulated and/or they may be cultured in a particular medium. The cells may be formulated in such a manner as to be suitable for delivery to a recipient without deleterious effects.

The medium in certain aspects can be prepared using a medium used for culturing animal cells as their basal medium, such as any of AIM V, X-VIVO-15, NeuroBasal, EGM2, TeSR, BME, BGJb, CMRL 1066, Glasgow MEM, Improved MEM Zinc Option, IMDM, Medium 199, Eagle MEM, αMEM, DMEM, Ham, RPMI-1640, and Fischer's media, as well as any combinations thereof, but the medium may not be particularly limited thereto as far as it can be used for culturing animal cells. Particularly, the medium may be xeno-free or chemically defined.

The medium can be a serum-containing or serum-free medium, or xeno-free medium. From the aspect of preventing contamination with heterogeneous animal-derived components, serum can be derived from the same animal as that of the stem cell(s). The serum-free medium refers to medium with no unprocessed or unpurified serum and accordingly, can include medium with purified blood-derived components or animal tissue-derived components (such as growth factors).

The medium may contain or may not contain any alternatives to serum. The alternatives to serum can include materials which appropriately contain albumin (such as lipid-rich albumin, bovine albumin, albumin substitutes such as recombinant albumin or a humanized albumin, plant starch, dextrans and protein hydrolysates), transferrin (or other iron transporters), fatty acids, insulin, collagen precursors, trace elements, 2-mercaptoethanol, 3′-thiolgiycerol, or equivalents thereto. The alternatives to serum can be prepared by the method disclosed in International Publication No. 98/30679, for example (incorporated herein in its entirety). Alternatively, any commercially available materials can be used for more convenience. The commercially available materials include KNOCKOUT™ Serum Replacement (KSR), Chemically-Defined Lipid Concentrate (GIBCO™), and GLUTAMAX™ (GIBCO™).

In certain embodiments, the medium may comprise one, two, three, four, five, six, seven, eight, nine, ten, 11, 12, 13, 14, 15, 16, 17, 18, 19, 20 or more of the following: Vitamins such as biotin; DL Alpha Tocopherol Acetate; DL Alpha-Tocopherol; Vitamin A (acetate); proteins such as BSA (bovine serum albumin) or human albumin, fatty acid free Fraction V; Catalase; Human Recombinant Insulin; Human Transferrin; Superoxide Dismutase; Other Components such as Corticosterone; D-Galactose; Ethanolamine HCl; Glutathione (reduced); L-Carnitine HCl; Linoleic Acid; Linolenic Acid; Progesterone; Putrescine 2HCl; Sodium Selenite; and/or T3 (triodo-I-thyronine). In specific embodiments, one or more of these may be explicitly excluded.

In some embodiments, the medium further comprises vitamins. In some embodiments, the medium comprises 1, 2, 3, 4, 5, 6, 7, 8, 9, 10, 11, 12, or 13 of the following (and any range derivable therein): biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, vitamin B12, or the medium includes combinations thereof or salts thereof. In some embodiments, the medium comprises or consists essentially of biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, and vitamin B12. In some embodiments, the vitamins include or consist essentially of biotin, DL alpha tocopherol acetate, DL alpha-tocopherol, vitamin A, or combinations or salts thereof. In some embodiments, the medium further comprises proteins. In some embodiments, the proteins comprise albumin or bovine serum albumin, a fraction of BSA, catalase, insulin, transferrin, superoxide dismutase, or combinations thereof. In some embodiments, the medium further comprises one or more of the following: corticosterone, D-Galactose, ethanolamine, glutathione, L-carnitine, linoleic acid, linolenic acid, progesterone, putrescine, sodium selenite, or triodo-I-thyronine, or combinations thereof. In some embodiments, the medium comprises one or more of the following: a B-27® supplement, xeno-free B-27™ supplement, GS21™ supplement, or combinations thereof. In some embodiments, the medium comprises or further comprises amino acids, monosaccharides, inorganic ions. In some embodiments, the amino acids comprise arginine, cystine, isoleucine, leucine, lysine, methionine, glutamine, phenylalanine, threonine, tryptophan, histidine, tyrosine, or valine, or combinations thereof. In some embodiments, the inorganic ions comprise sodium, potassium, calcium, magnesium, nitrogen, or phosphorus, or combinations or salts thereof. In some embodiments, the medium further comprises one or more of the following: molybdenum, vanadium, iron, zinc, selenium, copper, or manganese, or combinations thereof. In certain embodiments, the medium comprises or consists essentially of one or more vitamins discussed herein and/or one or more proteins discussed herein, and/or one or more of the following: corticosterone, D-Galactose, ethanolamine, glutathione, L-carnitine, linoleic acid, linolenic acid, progesterone, putrescine, sodium selenite, or triodo-I-thyronine, a B-27™ supplement, xeno-free B-27™ supplement, GS21™ supplement, an amino acid (such as arginine, cystine, isoleucine, leucine, lysine, methionine, glutamine, phenylalanine, threonine, tryptophan, histidine, tyrosine, or valine), monosaccharide, inorganic ion (such as sodium, potassium, calcium, magnesium, nitrogen, and/or phosphorus) or salts thereof, and/or molybdenum, vanadium, iron, zinc, selenium, copper, or manganese. In specific embodiments, one or more of these may be explicitly excluded.

The medium can also contain one or more externally added fatty acids or lipids, amino acids (such as non-essential amino acids), vitamin(s), growth factors, cytokines, antioxidant substances, 2-mercaptoethanol, pyruvic acid, buffering agents, and/or inorganic salts. In specific embodiments, one or more of these may be explicitly excluded.

One or more of the medium components may be added at a concentration of at least, at most, or about 0.1, 0.5, 1, 2, 3, 4, 5, 10, 15, 20, 25, 30, 35, 40, 45, 50, 55, 60, 65, 70, 75, 80, 85, 90, 95, 100, 150, 180, 200, 250 ng/L, ng/ml, μg/ml, mg/ml, or any range derivable therein.

In specific embodiments, the cells of the disclosure are specifically formulated. They may or may not be formulated as a cell suspension. In specific cases they are formulated in a single dose form. They may be formulated for systemic or local administration. In some cases the cells are formulated for storage prior to use, and the cell formulation may comprise one or more cryopreservation agents, such as DMSO (for example, in 5% DMSO). The cell formulation may comprise albumin, including human albumin, with a specific formulation comprising 2.5% human albumin. The cells may be formulated specifically for intravenous administration; for example, they are formulated for intravenous administration over less than one hour. In particular embodiments the cells are in a formulated cell suspension that is stable at room temperature for 1, 2, 3, or 4 hours or more from time of thawing.

In some embodiments, the cells of the disclosure further comprise one or more chimeric antigen receptors (CARs). Examples of tumor cell antigens to which a CAR may be directed include at least 5T4, 8H9, αvβ6 integrin, BCMA, B7-H3, B7-H6, CAIX, CA9, CD19, CD20, CD22, CD30, CD33, CD38, CD44, CD44v6, CD44v7/8, CD70, CD123, CD138, CD171, CEA, CSPG4, EGFR, EGFR family including ErbB2 (HER2), EGFRvIII, EGP2, EGP40, ERBB3, ERBB4, ErbB3/4, EPCAM, EphA2, EpCAM, folate receptor-a, FAP, FBP, fetal AchR, FR□, GD2, G250/CAIX, GD3, Glypican-3 (GPC3), Her2, IL-13Rα2, Lambda, Lewis-Y, Kappa, KDR, MAGE, MCSP, Mesothelin, Muc1, Muc16, NCAM, NKG2D Ligands, NY-ESO-1, PRAME, PSC1, PSCA, PSMA, ROR1, SP17, Survivin, TAG72, TEMs, carcinoembryonic antigen, HMW-MAA, AFP, CA-125, ETA, Tyrosinase, MAGE, laminin receptor, HPV E6, E7, BING-4, Calcium-activated chloride channel 2, Cyclin-B1, 9D7, EphA3, Telomerase, SAP-1, BAGE family, CAGE family, GAGE family, MAGE family, SAGE family, XAGE family, NY-ESO-1/LAGE-1, PAME, SSX-2, Melan-A/MART-1, GP100/pmel17, TRP-1/-2, P. polypeptide, MC1R, Prostate-specific antigen, β-catenin, BRCA1/2, CML66, Fibronectin, MART-2, TGF-βRII, or VEGF receptors (e.g., VEGFR2), for example. The CAR may be a first, second, third, or more generation CAR. The CAR may be bispecific for any two nonidentical antigens, or it may be specific for more than two nonidentical antigens.

In some embodiments, cells of the disclosure comprise one or more chimeric polypeptides. Cells may comprise a chimeric polypeptide of the disclosure together with a chimeric antigen receptor, T cell receptor, and/or other engineered receptor or molecule. In some embodiments, cells of the disclosure comprise 1, 2, 3, 4, or 5 chimeric polypeptides, or more.

### V. Chimeric Antigen Receptors

Certain embodiments of the disclosure are directed to Chimeric Antigen Receptors (CARs), cells comprising one or more CARs, and methods of use thereof.

### A. Signal Peptide

Polypeptides of the present disclosure may comprise a signal peptide. A “signal peptide” refers to a peptide sequence that directs the transport and localization of the protein within a cell, e.g., to a certain cell organelle (such as the endoplasmic reticulum) and/or the cell surface. In some embodiments, a signal peptide directs the nascent protein into the endoplasmic reticulum. This is essential if a receptor is to be glycosylated and anchored in the cell membrane. Generally, the signal peptide natively attached to the amino-terminal most component is used (e.g., in an scFv with orientation light chain-linker-heavy chain, the native signal of the light-chain is used).

In some embodiments, the signal peptide is cleaved after passage of the endoplasmic reticulum (ER), i.e., is a cleavable signal peptide. In some embodiments, a restriction site is at the carboxy end of the signal peptide to facilitate cleavage.

### B. Antigen Binding Domain

Polypeptides of the present disclosure may comprise one or more antigen binding domains. An “antigen binding domain” describes a region of a polypeptide capable of binding to an antigen under appropriate conditions. In some embodiments, an antigen binding domain is a single-chain variable fragment (scFv) based on one or more antibodies. In some embodiments, an antigen binding domain comprise a variable heavy (VH) region and a variable light (VL) region, with the VH and VL regions being on the same polypeptide. In some embodiments, the antigen binding domain comprises a linker between the VH and VL regions. A linker may enable the antigen binding domain to form a desired structure for antigen binding.

The variable regions of the antigen-binding domains of the polypeptides of the disclosure can be modified by mutating amino acid residues within the VH and/or VL CDR 1, CDR 2 and/or CDR 3 regions to improve one or more binding properties (e.g., affinity) of the antibody. The term “CDR” refers to a complementarity-determining region that is based on a part of the variable chains in immunoglobulins (antibodies) and T cell receptors, generated by B cells and T cells respectively, where these molecules bind to their specific antigen. Since most sequence variation associated with immunoglobulins and T cell receptors is found in the CDRs, these regions are sometimes referred to as hypervariable regions. Mutations may be introduced by site-directed mutagenesis or PCR-mediated mutagenesis and the effect on antibody binding, or other functional property of interest, can be evaluated in appropriate in vitro or in vivo assays. Preferably conservative modifications are introduced and typically no more than one, two, three, four or five residues within a CDR region are altered. The mutations may be amino acid substitutions, additions or deletions.

Framework modifications can be made to the antibodies to decrease immunogenicity, for example, by “backmutating” one or more framework residues to the corresponding germline sequence.

It is also contemplated that the antigen binding domain may be multi-specific or multivalent by multimerizing the antigen binding domain with VH and VL region pairs that bind either the same antigen (multi-valent) or a different antigen (multi-specific).

The binding affinity of the antigen binding region, such as the variable regions (heavy chain and/or light chain variable region), or of the CDRs may be at least 10⁻⁵M, 10⁻⁶M, 10⁻⁷M, 10⁻⁸M, 10⁻⁹M, 10⁻¹⁰M, 10⁻¹¹M, 10⁻¹²M, or 10⁻¹³M. In some embodiments, the KD of the antigen binding region, such as the variable regions (heavy chain and/or light chain variable region), or of the CDRs may be at least 10⁻⁵M, 10⁻⁶M, 10⁻⁷M, 10⁻⁸M, 10⁻⁹M, 10⁻¹⁰M, 10⁻¹¹M, 10⁻¹²M, or 10⁻¹³M (or any derivable range therein).

Binding affinity, KA, or KD can be determined by methods known in the art such as by surface plasmon resonance (SRP)-based biosensors, by kinetic exclusion assay (KinExA), by optical scanner for microarray detection based on polarization-modulated oblique-incidence reflectivity difference (OI-RD), or by ELISA.

In some embodiments, the polypeptide comprising the humanized binding region has equal, better, or at least 80, 81, 82, 83, 84, 85, 86, 87, 88, 89, 90, 91, 92, 93, 94, 95, 96, 97, 98, 99, 100, 101, 102, 103, 104, 104, 106, 106, 108, 109, 110, 115, or 120% binding affinity and/or expression level in host cells, compared to a polypeptide comprising a non-humanized binding region, such as a binding region from a mouse.

In some embodiments, the framework regions, such as FR1, FR2, FR3, and/or FR4 of a human framework can each or collectively have at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110,111, 112, 113, 114, 115, 116, 117, 118, 119, 120,121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 (or any derivable range therein) amino acid substitutions, contiguous amino acid additions, or contiguous amino acid deletions with respect to a mouse framework.

In some embodiments, the framework regions, such as FR1, FR2, FR3, and/or FR4 of a mouse framework can each or collectively have at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106,107, 108, 109, 110, 111, 112, 113, 114, 115, 116, 117, 118, 119, 120, 121,122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 (or any derivable range therein) amino acid substitutions, contiguous amino acid additions, or contiguous amino acid deletions with respect to a human framework.

The substitution may be at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 of FR1, FR2, FR3, or FR4 of a heavy or light chain variable region.

### C. Peptide Spacer

A peptide spacer, such as an extracellular spacer may link an antigen-binding domain to a transmembrane domain. In some embodiments, a peptide spacer is flexible enough to allow the antigen-binding domain to orient in different directions to facilitate antigen binding. In one embodiment, the spacer comprises the hinge region from IgG. In some embodiments, the spacer comprises or further comprises the CH2CH3 region of immunoglobulin and portions of CD3. In some embodiments, the CH2CH3 region may have L235E/N297Q or L235D/N297Q modifications, or at least 75%, at least 80%, at least 85%, at least 90%, at least 95%, at least 98%, or 100% amino acid sequence identity of the CH2CH3 region. In some embodiments, the spacer is from IgG4. An extracellular spacer may comprise a hinge region.

As used herein, the term “hinge” refers to a flexible polypeptide connector region (also referred to herein as “hinge region”) providing structural flexibility and spacing to flanking polypeptide regions and can consist of natural or synthetic polypeptides. A “hinge” derived from an immunoglobulin (e.g., IgG1) is generally defined as stretching from Glu216 to Pro230 of human IgG1 (Burton (1985) Molec. Immunol., 22: 161-206). Hinge regions of other IgG isotypes may be aligned with the IgG1 sequence by placing the first and last cysteine residues forming inter-heavy chain disulfide (S—S) bonds in the same positions. The hinge region may be of natural occurrence or non-natural occurrence, including but not limited to an altered hinge region as described in U.S. Pat. No. 5,677,425, incorporated by reference herein. The hinge region can include a complete hinge region derived from an antibody of a different class or subclass from that of the CH1 domain. The term “hinge” can also include regions derived from CD8 and other receptors that provide a similar function in providing flexibility and spacing to flanking regions.

The extracellular spacer can have a length of at least, at most, or exactly 4, 5, 6, 7, 8, 9, 10, 12, 15, 16, 17, 18, 19, 20, 20, 25, 30, 35, 40, 45, 50, 75, 100, 110, 119, 120, 130, 140, 150, 160, 170,180, 190, 200, 201, 202, 203, 204, 205, 206, 207, 208, 209, 210, 211, 212, 213, 214, 215, 216, 217, 218, 219, 220, 225, 226, 227, 228, 229, 230, 231, 232, 233, 234, 235, 236, 237, 238, 239, 240, 241, 242, 243, 244, 245, 246, 247, 248, 249, 250, 260, 270, 280, 290, 300, 325, 350, or 400 amino acids (or any derivable range therein). In some embodiments, the extracellular spacer consists of or comprises a hinge region from an immunoglobulin (e.g., IgG). Immunoglobulin hinge region amino acid sequences are known in the art; see, e.g., Tan et al., (1990) Proc. Natl. Acad. Sci. USA 87: 162; and Huck et al., (1986) Nucl. Acids Res.

The length of an extracellular spacer may have effects on the CAR's signaling activity and/or the CAR-T cells' expansion properties in response to antigen-stimulated CAR signaling. In some embodiments, a shorter spacer such as less than 50, 45, 40, 30, 35, 30, 25, 20, 15, 14, 13, 12, 11, or 10 amino acids is used. In some embodiments, a longer spacer, such as one that is at least 50, 60, 70, 80, 90, 100, 110, 120, 130, 140, 150, 200, 201, 202, 203, 204, 205, 206, 207, 208, 209, 210, 211, 212, 213, 214, 215, 216, 217, 218, 219, 220, 225, 226, 227, 228, 229, 230, 231, 232, 233, 234, 235, 236, 237, 238, 239, 240, 241, 242, 243, 244, 245, 246, 247, 248, 249, 250, 260, 270, 280, or 290 amino acids may have the advantage of increased expansion in vivo or in vitro.

When the extracellular spacer comprises multiple parts, there may be anywhere from 0-50 amino acids in between the various parts. For example, there may be at least, at most, or exactly 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50 amino acids (or any derivable range therein) between the hinge and the CH2 or CH3 region or between the CH2 and CH3 region when both are present. In some embodiments, the extracellular spacer consists essentially of a hinge, CH2, and/or CH3 region, meaning that the hinge, CH2, and/or CH3 region is the only identifiable region present and all other domains or regions are excluded, but further amino acids not part of an identifiable region may be present.

### D. Transmembrane Domain

Polypeptides of the present disclosure may comprise a transmembrane domain. In some embodiments, a transmembrane domain is a hydrophobic alpha helix that spans the membrane. Different transmembrane domains may result in different receptor stability.

In some embodiments, the transmembrane domain is interposed between the extracellular spacer and the cytoplasmic region. In some embodiments, the transmembrane domain is interposed between the extracellular spacer and one or more costimulatory regions. In some embodiments, a linker is between the transmembrane domain and the one or more costimulatory regions.

Any transmembrane domain that provides for insertion of a polypeptide into the cell membrane of a eukaryotic (e.g., mammalian) cell may be suitable for use. In some embodiments, the transmembrane domain is derived from CD28, CD8, CD4, CD3-zeta (CD3ζ), CD134, or CD7.

### E. Cytoplasmic Region

After antigen recognition, receptors of the present disclosure may cluster and a signal transmitted to the cell through the cytoplasmic region. In some embodiments, the costimulatory domains described herein are part of the cytoplasmic region. In some embodiments, the cytoplasmic region comprises an intracellular signaling domain. An intracellular signaling domain may comprise a primary signaling domain and one or more costimulatory domains.

Cytoplasmic regions and/or costimulatory regions suitable for use in the polypeptides of the disclosure include any desired signaling domain that provides a distinct and detectable signal (e.g., increased production of one or more cytokines by the cell; change in transcription of a target gene; change in activity of a protein; change in cell behavior, e.g., cell death; cellular proliferation; cellular differentiation; cell survival; modulation of cellular signaling responses; etc.) in response to activation by way of binding of the antigen to the antigen binding domain. In some embodiments, the cytoplasmic region includes at least one (e.g., one, two, three, four, five, six, etc.) ITAM motif as described herein. In some embodiments, the cytoplasmic region includes DAP10/CD28 type signaling chains.

Cytoplasmic regions suitable for use in the polypeptides of the disclosure include immunoreceptor tyrosine-based activation motif (ITAM)-containing intracellular signaling polypeptides. An ITAM motif is YX1X2(L/I), where X1 and X2 are independently any amino acid. In some cases, the cytoplasmic region comprises 1, 2, 3, 4, or 5 ITAM motifs. In some cases, an ITAM motif is repeated twice in an endodomain, where the first and second instances of the ITAM motif are separated from one another by 6 to 8 amino acids, e.g., (YX1X2(L/I))(X3)n(YX1X2(L/I)), where n is an integer from 6 to 8, and each of the 6-8 X3 can be any amino acid.

A suitable cytoplasmic region may be an ITAM motif-containing portion that is derived from a polypeptide that contains an ITAM motif. For example, a suitable cytoplasmic region can be an ITAM motif-containing domain from any ITAM motif-containing protein. Thus, a suitable endodomain need not contain the entire sequence of the entire protein from which it is derived. Examples of suitable ITAM motif-containing polypeptides include, but are not limited to: DAP12, DAP10, FCER1G (Fc epsilon receptor I gamma chain); CD3D (CD3 delta); CD3E (CD3 epsilon); CD3G (CD3 gamma); CD3-zeta; and CD79A (antigen receptor complex-associated protein alpha chain).

Exemplary cytoplasmic regions are known in the art. The cytoplasmic regions shown below also provide examples of regions that may be incorporated in a CAR of the disclosure:

In some embodiments, a suitable cytoplasmic region can comprise an ITAM motif-containing portion of the full length DAP12 amino acid sequence. In some embodiments, the cytoplasmic region is derived from FCER1G (also known as FCRG; Fc epsilon receptor I gamma chain; Fc receptor gamma-chain; fc-epsilon RI-gamma; fcRgamma; fceRI gamma; high affinity immunoglobulin epsilon receptor subunit gamma; immunoglobulin E receptor, high affinity, gamma chain; etc.). In some embodiments, a suitable cytoplasmic region can comprise an ITAM motif-containing portion of the full length FCER1G amino acid sequence.

In some embodiments, the cytoplasmic region is derived from T cell surface glycoprotein CD3 delta chain (also known as CD3D; CD3-DELTA; T3D; CD3 antigen, delta subunit; CD3 delta; CD36; CD3d antigen, delta polypeptide (TiT3 complex); OKT3, delta chain; T cell receptor T3 delta chain; T cell surface glycoprotein CD3 delta chain; etc.). In some embodiments, a suitable cytoplasmic region can comprise an ITAM motif-containing portion of the full length CD3 delta amino acid sequence. In some embodiments, the cytoplasmic region is derived from T cell surface glycoprotein CD3 epsilon chain (also known as CD3e, CD3F; T cell surface antigen T3/Leu-4 epsilon chain, T cell surface glycoprotein CD3 epsilon chain, AI504783, CD3, CD3-epsilon, T3e, etc.). In some embodiments, a suitable cytoplasmic region can comprise an ITAM motif-containing portion of the full length CD3 epsilon amino acid sequence. In some embodiments, the cytoplasmic region is derived from T cell surface glycoprotein CD3 gamma chain (also known as CD3G, CD3γ, T cell receptor T3 gamma chain, CD3-GAMMA, T3G, gamma polypeptide (TiT3 complex), etc.). In some embodiments, a suitable cytoplasmic region can comprise an ITAM motif-containing portion of the full length CD3 gamma amino acid sequence. In some embodiments, the cytoplasmic region is derived from T cell surface glycoprotein CD3 zeta chain (also known as CD3Z, CD3γ, T cell receptor T3 zeta chain, CD247, CD3-ZETA, CD3H, CD3Q, T3Z, TCRZ, etc.). In some embodiments, a suitable cytoplasmic region can comprise an ITAM motif-containing portion of the full length CD3 zeta amino acid sequence.

In some embodiments, the cytoplasmic region is derived from CD79A (also known as B-cell antigen receptor complex-associated protein alpha chain; CD79a antigen (immunoglobulin-associated alpha); MB-1 membrane glycoprotein; ig-alpha; membrane-bound immunoglobulin-associated protein; surface IgM-associated protein; etc.). In some embodiments, a suitable cytoplasmic region can comprise an ITAM motif-containing portion of the full length CD79A amino acid sequence.

### F. Costimulatory Region

Non-limiting examples of suitable costimulatory regions, such as those included in the cytoplasmic region, include, but are not limited to, polypeptides from 4-1BB (CD137), CD28, ICOS, OX-40, BTLA, CD27, CD30, GITR, and HVEM.

A costimulatory region may have a length of at least, at most, or exactly 20, 25, 30, 35, 40, 50, 60, 70, 80, 90, 100, 150, 200, or 300 amino acids or any range derivable therein. In some embodiments, the costimulatory region is derived from an intracellular portion of the transmembrane protein 4-1BB (also known as TNFRSF9; CD137; CDwl37; ILA; etc.). In some embodiments, the costimulatory region is derived from an intracellular portion of the transmembrane protein CD28 (also known as Tp44). In some embodiments, the costimulatory region is derived from an intracellular portion of the transmembrane protein ICOS (also known as AILIM, CD278, and CVID1). In some embodiments, the costimulatory region is derived from an intracellular portion of the transmembrane protein OX-40 (also known as TNFRSF4, RP5-902P8.3, ACT35, CD134, OX40, TXGP1L). In some embodiments, the costimulatory region is derived from an intracellular portion of the transmembrane protein BTLA (also known as BTLA1 and CD272). In some embodiments, the costimulatory region is derived from an intracellular portion of the transmembrane protein CD27 (also known as S 152, T14, TNFRSF7, and Tp55). In some embodiments, the costimulatory region is derived from an intracellular portion of the transmembrane protein CD30 (also known as TNFRSF8, D1S166E, and Ki-1). In some embodiments, the costimulatory region is derived from an intracellular portion of the transmembrane protein GITR (also known as TNFRSF18, RP5-902P8.2, AITR, CD357, and GITR-D). In some embodiments, the costimulatory region derived from an intracellular portion of the transmembrane protein HVEM (also known as TNFRSF14, RP3-395M20.6, ATAR, CD270, HVEA, HVEM, LIGHTR, and TR2).

### G. Detection Peptides

In some embodiments, the polypeptides described herein may further comprise a detection peptide. Various suitable detection peptides are known in the art and contemplated herein.

### H. Peptide Linkers

In some embodiments, the polypeptides of the disclosure include peptide linkers (sometimes referred to as a linker). A peptide linker may be used to separate any of the peptide domain/regions described herein. As an example, a linker may be between the signal peptide and the antigen binding domain, between the VH and VL of the antigen binding domain, between the antigen binding domain and the peptide spacer, between the peptide spacer and the transmembrane domain, flanking the costimulatory region or on the N- or C-region of the costimulatory region, and/or between the transmembrane domain and the endodomain. The peptide linker may have any of a variety of amino acid sequences. Domains and regions can be joined by a peptide linker that is generally of a flexible nature, although other chemical linkages are not excluded. A linker can be a peptide of between about 6 and about 40 amino acids in length, or between about 6 and about 25 amino acids in length. These linkers can be produced by using synthetic, linker-encoding oligonucleotides to couple the proteins.

Peptide linkers with a degree of flexibility can be used. The peptide linkers may have virtually any amino acid sequence, bearing in mind that suitable peptide linkers will have a sequence that results in a generally flexible peptide. The use of small amino acids, such as glycine and alanine, are of use in creating a flexible peptide. The creation of such sequences is routine to those of skill in the art.

Suitable linkers can be readily selected and can be of any suitable length, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

Suitable linkers can be readily selected and can be of any of a suitable of different lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

### VI. Cells

Certain embodiments relate to cells comprising polypeptides or nucleic acids of the disclosure. In some embodiments the cell is an immune cell or a T cell. “T cell” includes all types of immune cells expressing CD3 including T-helper cells, invariant natural killer T (iNKT) cells, cytotoxic T cells, T-regulatory cells (Treg) gamma-delta T cells, natural-killer (NK) cells, and neutrophils. The T cell may refer to a CD4+ or CD8+ T cell.

Suitable mammalian cells include primary cells and immortalized cell lines. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), human embryonic kidney (HEK) 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RATI cells, mouse L cells (ATCC No. CCLI.3), HLHepG2 cells, Hut-78, Jurkat, HL-60, NK cell lines (e.g., NKL, NK92, and YTS), and the like.

In some instances, the cell is not an immortalized cell line, but is instead a cell (e.g., a primary cell) obtained from an individual. For example, in some cases, the cell is an immune cell obtained from an individual. As an example, the cell is a T lymphocyte obtained from an individual. As another example, the cell is a cytotoxic cell obtained from an individual. As another example, the cell is a stem cell (e.g., peripheral blood stem cell) or progenitor cell obtained from an individual.

### VII. Cytokines

In some embodiments, any cells encompassed herein may express one or more heterologous cytokines. In specific embodiments, the cells are engineered to express one or more heterologous cytokines and/or are engineered to upregulate normal expression of one or more heterologous cytokines. The cells may or may not be transduced or transfected for one or more cytokines on the same vector as other genes. In some cases, the cells also express one or more heterologous proteins, including chimeric proteins of any kind, in addition to one or more cytokines. As one example, the cells may express a chimeric antigen receptor.

One or more cytokines may be co-expressed from a vector as a separate polypeptide from other proteins, including a chimeric polypeptide and/or a CAR. Interleukin-15 (IL-15), for example, is tissue restricted and only under pathologic conditions is it observed at any level in the serum, or systemically. IL-15 possesses several attributes that are desirable for adoptive therapy. IL-15 is a homeostatic cytokine that induces development and cell proliferation of natural killer cells, promotes the eradication of established tumors via alleviating functional suppression of tumor-resident cells, and inhibits activation-induced cell death (AICD). In addition to IL-15, other cytokines are envisioned. These include, but are not limited to, cytokines, chemokines, and other molecules that contribute to the activation and proliferation of cells used for human application. NK cells expressing IL-15 are capable of continued supportive cytokine signaling, which is useful for their survival post-infusion.

In specific embodiments, the cells expresses one or more exogenously provided cytokines. As one example, the cytokine is IL-15, IL-12, IL-2, IL-18, IL-21, IL-23, GMCSF, or a combination thereof. In addition, or alternatively, the cytokine(s) may be exogenously provided to the cells. In an alternative case, an endogenous cytokine in the cell is upregulated upon manipulation of regulation of expression of the endogenous cytokine, such as genetic recombination at the promoter site(s) of the cytokine. In cases wherein the cytokine is provided on an expression construct to the cell, the cytokine may be encoded from the same vector as a other heterologous proteins.

In some embodiments, a heterologous protein utilized in the cells is a fusion of a cytokine and at least part of its receptor, including part or all of an extracellular domain of its receptor. In a specific case, part or all of IL-15 is fused to part or all of the IL-15Ra receptor, including part or all of the extracellular domain of the IL-15Ra receptor, such as the sushi domain.

### VIII. General Pharmaceutical Compositions

In some embodiments, pharmaceutical compositions are administered to a subject. Different aspects may involve administering an effective amount of a composition to a subject. In some embodiments, a cellular therapy (e.g., CAR T cells, CAR NK cells, TCR T cells, etc.) is administered to the subject to protect against or treat a condition (e.g., cancer). Additionally, such compositions can be administered in combination with an additional therapeutic agent (e.g., a chemotherapeutic, an immunotherapeutic, a biotherapeutic, etc.). Such compositions will generally be dissolved or dispersed in a pharmaceutically acceptable carrier or aqueous medium.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

The active compounds can be formulated for parenteral administration, e.g., formulated for injection via the intravenous, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The proteinaceous compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions will typically be via any common route. This includes, but is not limited to oral, or intravenous administration. Alternatively, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients.

Upon formulation, solutions will be administered in a manner compatible with the dosage formulation and in such amount as is therapeutically or prophylactically effective. The formulations are easily administered in a variety of dosage forms, such as the type of injectable solutions described above.

### IX. Chimeric Polypeptides as Transduction Markers and/or Safety Switches

Embodiments of the disclosure include chimeric polypeptides that may be utilized as transduction markers and/or safety switches. Safety strategies, e.g., may be utilized with cellular therapies as a means to overcome toxicity should it occur. Examples present in the art include Herpes simplex virus thymidine kinase (HSV-tk)/ganciclovir, inducible caspase 9, and the truncated EGFR gene (tEGFR). The EGFR gene mainly uses the antibody-dependent cellular cytotoxicity (ADCC) mechanism to eliminate therapeutic cells, and ADCC requires the presence of NK cells and other effector cells that could limit efficacy of such a safety switch in vivo. Ideally a safety switch would utilize ADCC or antibody-drug conjugate (ADC) mechanisms to eliminate therapeutic cells in vivo. The ADC approach does not require effector cells for elimination of the therapeutic cells in vivo and therefore would be expected to be more effective in vivo. In addition, a tEGRF safety switch may have limited or no efficacy in patients who were recently treated with lymphodepleting chemotherapy, for example. The present disclosure provides solutions to needs in the art of safety for cellular therapies.

Embodiments of the disclosure include methods and compositions related to cellular therapies having a reduced risk for deleterious effects compared to the cellular therapies that are not likewise produced. In particular embodiments, therapeutic cells of the disclosure express at least one protein marker that can also be used as a safety switch, for example in the event that the cellular therapy becomes toxic to a recipient individual. In some cases, the protein is utilized as a marker and not a safety switch, in some cases the protein is used as a safety switch and not a marker, and in other cases the protein is utilized as both a marker and a safety switch. The therapeutic cells may or may not be monitored using the marker prior to onset of any toxicity. The marker/safety switch may also be useful to monitor production of cells and/or use of the cells.

In particular embodiments, the chimeric polypeptide is utilized both as a marker for particular cells but also as a safety switch to kill the cells, if needed. In some embodiments, the chimeric polypeptide is present in cells in which it is not normally expressed and/or the chimeric polypeptide is present in cells as a heterologous protein of any kind, including, in some cases, as a fusion protein. In specific embodiments, the chimeric polypeptide fusion proteins comprising at least the extracellular domain of a particular protein are utilized both as a marker for cells that express it and also, if desired, as a target to kill the cells that express it. In specific cases, the chimeric polypeptide is present as a fusion protein in which a cytoplasmic domain is not native to any part of the remainder of the protein (although the transmembrane domain of the fusion protein may or may not be the transmembrane domain natively found with the extracellular domain), and in some aspects the cytoplasmic domain is the cytoplasmic domain of a cellular receptor, such as an internalization cytoplasmic domain. In specific cases, the cytoplasmic domain of the chimeric polypeptide fusion protein is from CD30, B cell maturation antigen (BCMA), tumor-associated calcium signal transducer 2 (trop-2), CD317, CD3gamma, CD4, CD79b, CD19, CD22, CD25, CD33, or a combination thereof.

Methods of the disclosure include at least methods of identifying cells that express the chimeric polypeptide (such as a polypeptide that comprises at least part of a particular extracellular domain to an extent that an agent that binds the extracellular domain, such as an antibody, is able to bind it). The identifying of the cells using the particular protein can be for any reason, such as quality of production of cells expressing it, monitoring location of the cells, and/or determining quantity of the cells, and so forth.

In specific embodiments, the chimeric polypeptide comprises part or all of the CD30 extracellular domain. In specific cases, cells of a cellular therapy are controlled by targeting of CD30 being expressed on the cells. Control of the CD30-positive cells allow for them to be inhibited, including killed, for example should a recipient of the cells exhibit any indication that the cell therapy has become deleterious in any manner, such as exhibiting toxicity to the individual. In some embodiments, the CD30-positive cells are of any kind and express a heterologous CD30 protein of any kind, including a chimeric fusion protein. In other embodiments, the natural machinery of the cells causes the cells to express CD30 under non-native conditions. For example, CD30 may be expressed following transfection or transduction of one or more heterologous genes, including particular combinations of heterologous genes. In specific cases, transfection or transduction of BCL6 and BCL2L1 in cells results in expression of CD30 (including in cells that do not normally express CD30), and in such cases the CD30 may be used as a safety switch (or a marker). In specific embodiments, when a recipient individual exhibits toxicity to CD30-positive cell therapy, the individual is provided an effective amount of one or more agents that target CD30, such as antibodies or antibody-drug conjugates and including monoclonal antibodies; chimeric antigen receptor-expressing immune cells (T cells ((including αβ or γδ), NK cells, NKT cells, monocytes, macrophages, B cells, mesenchymal stem cells (MSC) cells, hematopoietic stem cells (HSC), hematopoietic cells, induced pluripotent stem cells (iPSC) or their derivatives, mixtures thereof, derivatives thereof) targeting CD30; CD30/CD3 bispecific antibodies; or CD30/CD16 bispecific antibodies.

Embodiments of the disclosure include expression constructs comprising sequence that encodes a particular chimeric polypeptide, wherein the chimeric polypeptide comprises at least part of a particular extracellular domain fused to an intracellular domain that comprises endocytosis or internalization activity. In specific cases, the cytoplasmic domain of the chimeric polypeptide fusion protein is from CD30, B cell maturation antigen (BCMA), tumor-associated calcium signal transducer 2 (trop-2), CD317, CD3gamma, CD4, CD79b, CD19, CD22, CD25, CD33, or a combination thereof. In specific cases, the chimeric polypeptide may or may not comprise a particular transmembrane domain, such as a transmembrane domain that is an alpha or beta chain of the T cell receptor or a transmembrane domain from CD28, CD3E, CD45, CD4, CD5, CD8, CD9, CD16, CD22, CD30, CD33, CD37, CD64, CD80, CD86, CD123, CD134, CD137 or CD154. In specific embodiments, the fusion protein comprises SEQ ID NO:47, and the sequence that encodes the fusion protein may comprise SEQ ID NO:48. Any expression construct encompassed herein may be in a vector, including a viral vector or non-viral vector.

Specific embodiments of the disclosure include isolated cells comprising any expression construct encompassed herein, such as an immune cell. The cells may be αβ T cells, γδ T cells, NK cells, NKT cells, monocytes, macrophages, B cells, mesenchymal stem cell (MSC) cells, hematopoietic stem cells (HSC), hematopoietic cells, iPSCs, or a mixture thereof. In some cases, the cell expresses one or more heterologous proteins other than the chimeric polypeptide. Any heterologous protein may be a therapeutic protein, cytokine, fusion of a cytokine and cytokine receptor, safety switch, or a mixture thereof. In specific cases, the therapeutic protein is an engineered antigen receptor, antibody, and so forth. The engineered antigen receptor may target a cancer antigen. The engineered antigen receptor may be a chimeric antigen receptor, T cell receptor, or B-cell receptor. In specific embodiments of the cells, the chimeric polypeptide and one or more heterologous proteins are expressed from the same vector, although in other cases the chimeric polypeptide and one or more heterologous proteins are expressed from different vectors. The cell may express heterologous BCL6 and one or more Bcl2 family genes. Embodiments of the disclosure include isolated populations of any one of the cells encompassed herein comprised in a suitable medium. The population may be housed in a depository and/or cryopreserved.

Specific embodiments of the disclosure include methods of identifying CD30-positive cells that are transduced or transfected with (1) a heterologous CD30 gene; (2) a CD30 fusion protein comprising at least part of the CD30 extracellular domain; or (3) a combination of heterologous BCL6 and one or more Bcl2 family genes, comprising the steps of: transducing or transfecting cells with (1) a heterologous CD30 gene; (2) a CD30 fusion protein comprising at least part of the CD30 extracellular domain; or (3) a combination of heterologous BCL6 and one or more Bcl2 family genes; exposing the cells to an effective amount of an agent that binds CD30; and directly or indirectly detecting the binding of the agent to CD30 on the surface of the cells. The exposing and detecting steps may occur during and/or after manufacture of the cells. The method may further comprise the step of manufacturing the cells. In specific cases, the method is further defined as: transfecting or transducing immune cells with the CD30 fusion protein; exposing the immune cells to an effective amount of an antibody or antibody-drug conjugate that binds CD30; and directly or indirectly detecting the binding of the antibody or antibody-drug conjugate to CD30 on the surface of the cells. The method may be further defined as: transfecting or transducing immune cells with the heterologous BCL6 and one or more Bcl2 family genes; exposing the immune cells to an effective amount of an antibody or antibody-drug conjugate that binds CD30; and directly or indirectly detecting the binding of the antibody or antibody-drug conjugate to CD30 on the surface of the cells. In specific cases, the method occurs in vitro, although in some cases at least part of the method occurs in vivo. The cells may express one or more heterologous proteins other than the CD30 fusion protein, such as a therapeutic protein, cytokine, fusion of a cytokine and cytokine receptor, safety switch, or a mixture thereof. The therapeutic protein may be an engineered antigen receptor. In specific embodiments, the method further comprises the step of transfecting or transforming the immune cells with a heterologous protein other than the heterologous CD30 or the CD30 fusion protein; or the BCL6 and one or more Bcl2 family genes. In some embodiments, the CD30 fusion protein and the heterologous protein other than the CD30 fusion protein or the BCL6 and one or more Bcl2 family genes are expressed from the same vector. The CD30 fusion protein and the heterologous protein other than the CD30 fusion protein or the BCL6 and one or more Bcl2 family genes may be expressed from different vectors.

In one embodiments, there is a method of manufacturing immune cells for adoptive cell therapy, comprising the steps of: (a) transducing or transfecting immune cells with (1) heterologous CD30 protein; (2) a CD30 fusion protein; or (3) a combination of heterologous BCL6 and one or more Bcl2 family genes, wherein the immune cells respectively express (1) CD30; (2) the CD30 fusion protein; or (3) CD30; and (b) transducing or transfecting the immune cells with one or more therapeutic proteins. In specific embodiments, step (a) occurs prior to, at the same time as, or subsequent to step (b). The transforming or transfecting in step (a) may be the same transforming or transfecting in step (b). In specific embodiments, (1), (2), or (3) are on the same vector as the therapeutic protein, although (1), (2), or (3) may be on a different vector as the therapeutic protein. In specific embodiments, following step (a), immune cells from the method are analyzed for the presence of CD30 expressed on the surface of the immune cells, such as by flow cytometry, polymerase chain reaction, or a combination thereof. In some cases, the method further comprising the step of administering immune cells produced from the method to an individual in need thereof. In any case, the immune cells may be monitored in the individual, such as using an agent that binds CD30 (an antibody or antibody-drug conjugate, for example). In specific embodiments, the individual exhibits one or more deleterious effects from the immune cells, and the individual is administered an effective amount of an agent that binds CD30. The individual may exhibit toxicity from the immune cells, and the individual may be administered an effective amount of an agent that binds CD30. The individual may exhibit graft-versus-host disease (GVHD) from the immune cells, and the individual may be administered an effective amount of an agent that binds CD30. The individual may no longer be in need of the immune cells, and the individual may be administered an effective amount of an agent that binds CD30.

Embodiments of the disclosure include methods of reducing or preventing one or more deleterious effects from a cellular therapy in an individual, comprising the step of targeting the extracellular domain of a chimeric polypeptide expressed on the surface of cells of the cellular therapy. The method may be further defined as administering to the individual an effective amount of one or more agents that bind the extracellular domain of the chimeric polypeptide expressed on the cells, such as an antibody (such as a monoclonal antibody) or antibody-drug conjugate. Examples of deleterious effects include GVHD, cytokine release syndrome, or immune effector cell-associated neurotoxicity syndrome. In cases wherein a polypeptide comprises the extracellular domain of CD30, the expressed CD30 extracellular domain may or may not also include the entire CD30 protein. In specific cases, the CD30 is a fragment of the entire CD30 protein that comprises at least part of the extracellular domain. The CD30 may be expressed naturally on the immune cells. In some cases, the CD30 is heterologously expressed naturally on the immune cells. The CD30 may be expressed on the cells as a result of the cells expressing heterologous BCL6 and one or more Bcl2 family genes. The CD30 may be a CD30 fusion protein, such as one that comprises at least part of the CD30 extracellular domain fused to an intracellular domain that comprises endocytosis or internalization activity. Examples of intracellular domains include those from B-cell maturation antigen (BCMA), trop-2, CD317, CD3gamma, CD4, CD79b, or a combination thereof. The CD30 fusion protein may comprise the CD30 transmembrane domain. In specific cases, the fusion protein comprises SEQ ID NO:47. The sequence that encodes the fusion protein may comprise SEQ ID NO:48.

In specific cases, prior to any targeting step, the cells of the individual may be monitored in vivo, such as using one or more agents that target a chimeric polypeptide thereof. In particular embodiments, the agent that targets the chimeric polypeptide is an antibody used in a low enough amount not to inhibit the therapeutic cells. The cellular therapy may be allogeneic or autologous with respect to the individual. The cellular therapy may comprise immune cells that express one or more heterologous proteins, including a therapeutic protein, cytokine, fusion of a cytokine and cytokine receptor, safety switch, or a mixture thereof. The therapeutic protein may comprise one or more engineered antigen receptors, including a chimeric antigen receptor, a T cell receptor, or both being expressed by the cell. The cells may express a chimeric polypeptide and a chimeric antigen receptor.

Embodiments of the disclosure include methods of inhibiting the activity of cells, comprising the step of exposing cells that are transduced or transfected with a combination of heterologous BCL6 and one or more Bcl2 family genes to an effective amount of an agent that binds CD30. In specific embodiments, the inhibiting of activity is further defined as inducing apoptosis of the cells. The method may further comprise detecting binding of the agent to CD30 expressed on the cells. In specific cases, the method further comprises transducing or transfecting cells of any kind encompassed herein with the heterologous BCL6 and with one or more Bcl2 family genes. The one or more Bcl2 family genes may be BCL2L1.

Embodiments of the disclosure include methods of inhibiting the activity of cells, comprising the step of exposing cells that are transduced or transfected with a CD30 fusion protein comprising at least part of the CD30 extracellular domain to an effective amount of an agent that binds CD30. In specific embodiments, the inhibiting of activity is further defined as inducing apoptosis of the cells. The method may further comprise detecting binding of the agent to CD30 expressed on the cells.

### X. CD30-Positive Cells and Related Compositions

CD30 is also known as TNFRSF8, D1S166E, Ki-1, tumor necrosis factor receptor superfamily member 8, and TNF receptor superfamily member 8. The present disclosure concerns compositions comprising CD30-positive cells that reduce the risk of a cellular therapy from being toxic to a recipient individual and/or that allow monitoring of production and/or monitoring of location of the cells. The CD30-positive cells produced using methods of the disclosure have a reduced risk for toxicity compared to cells that were not produced using methods of the disclosure. The expression of the extracellular domain of the CD30 protein on the surface of the cells allows the protein to be targeted with one or more agents that recognize the domain on the surface and that directly or indirectly results in inhibition of the cells, including death of the cells.

In specific embodiments, CD30-positive cells are utilized as a marker of any kind and as a safety switch, including for cells having the potential of becoming toxic to a recipient individual. Embodiments of the disclosure include methods and compositions in which CD30 is effective as either or both of a transduction marker and a safety switch. The cellular therapy comprises CD30-positive cells that in specific embodiments are themselves immune cells that may or may not comprise modifications to express one or more heterologous genes.

CD30-positive cells include those in which CD30 is naturally expressed on the surface of the cell. Alternatively, the cells may be modified by the hand of man to express part or all of CD30 and in some cases the cells in the absence of such modification would not express CD30. The CD30 may or may not be wild-type. In particular embodiments, part, but not all, of the CD30 protein is expressed in a cell, and when less than all of the CD30 protein is utilized, it includes at least part of the CD30 extracellular domain if not all of the extracellular domain. In specific embodiments, the CD30 transmembrane domain is utilized, whereas in other cases it is not. In specific cases, at least part of CD30 is utilized in a fusion protein, including one in which the CD30 fusion protein is expressed on the surface of the cells. In such cases, the CD30 fusion protein comprises as least part if not all of the CD30 extracellular domain. In specific cases, enough of the CD30 extracellular domain is included in the fusion protein such that an antibody that recognizes the CD30 extracellular domain is able to bind it at the appropriate epitope.

The cells may also have a unique capacity for expressing CD30 as a cell surface marker following modification by the hand of man, such as following transfection or transduction of one or more heterologous genes that are not CD30 For certain cells, including at least T cells (including □□ or □□), NK cells, NKT cells, monocytes, macrophages, B cells, mesenchymal stem cells (MSC) cells, hematopoietic stem cells (HSC), hematopoietic cells, induced pluripotent stem cells (iPSC) or their derivatives, mixtures thereof, derivatives thereof), transfection or transduction of one or more heterologous genes results in expression of CD30 when CD30 normally would not be expressed. The heterologous gene(s) may or may not be unrelated to pathways that utilized CD30, and in specific cases the heterologous genes are the BCL6 and BCL2L1 genes or related genes thereof. In such cases, this allows CD30 to be utilized as a transduction marker for successful transfection or transduction of the cells with the one or more heterologous genes.

CD30 (or derivatives thereof) as a membrane bound protein may be utilized in a variety of therapeutic immune cellular products. In some cases, the immune cells include at least T cells (including □□ or □□), NK cells, NKT cells, monocytes, macrophages, B cells, mesenchymal stem cells (MSC) cells, hematopoietic stem cells (HSC), hematopoietic cells, induced pluripotent stem cells (iPSC) or their derivatives, mixtures thereof, derivatives thereof, etc.

In specific cases, the membrane-bound protein comprises part or all the extracellular domain of CD30 and/or the intracellular domain of CD30. In some cases, the membrane-bound protein is the full-length wildtype CD30 protein. The CD30 in the cell may comprise the CD30 extracellular domain and the transmembrane domain but not the CD30 intracellular domain. The CD30 in the cell may comprise the CD30 extracellular domain and the CD30 intracellular domain but not the CD30 transmembrane domain. The membrane-bound CD30 protein may comprise a cytoplasmic tail (that has at least an internalization motif, which means that the tail can interact with the clathrin-mediated intracellular trafficking complex to internalize the anti-CD30 antibody bound with CD30) that comprises one or more internalization cytoplasmic tails of one or more other membrane-bound receptors. Consensus sequences associated with clathrin-dependent endocytic sorting signals are known in the art (Traub, Molecular Cell Biology, Vol. 10, pp. 583-596, 2009). In some cases the cytoplasmic tail is the cytoplasmic portion of a surface bound protein. In specific embodiments, the cytoplasmic tail is the cytoplasmic tail of CD30, BCMA, trop-2, CD317, CD3gamma, CD4, CD79b, CD19, CD22, CD25, CD33, etc.

In one embodiment, the CD30 is co-expressed in the cells with one or more heterologous proteins in the cells. The heterologous protein may or may not be a therapeutic protein, including one that has therapeutic efficacy itself and/or one that imparts therapeutic efficacy to the CD30-positive cells that express it. The cells may be manufactured at the same time or at different times to express CD30 and the heterologous protein. In some embodiments, cells that may or may not be CD30-positive are modified to express one or more heterologous proteins and then are stored in a repository, including cryopreserved. Upon need, the cells may be thawed and further modified to express CD30, to express a CD30 fusion protein, or to be transformed/transfected to express one or more heterologous genes that then upregulate expression of CD30 in the cells. In other cases, cells that express CD30, that express a CD30 fusion protein, or that are transformed/transfected to express one or more heterologous genes that then upregulate expression of CD30 in the cells are modified to express one or more heterologous proteins following thawing of the cells from a cryopreserved condition in a repository.

In particular embodiments, for any of the CD30-positive cells the heterologous gene(s) may be one or more engineered antigen receptors, including chimeric antigen receptors or TCR-expressing cells. In such cases, any engineered antigen receptor may target one or more antigens, including one or more cancer antigens. The engineered antigen receptor may be directed to target single or multiple antigens of interest, including cancer antigens.

In specific cases, the CD30-positive cells are autologous or allogenic cells (with respect to an individual) that may or may not express one or more heterologous proteins.

In some embodiments, the CD30-positive cells express an additional safety switch other than CD30 or CD30 fusion proteins, including Herpes simplex virus thymidine kinase (HSV-tk)/ganciclovir, iCaspase9, tEGFR, synNotch, combinatorial target-antigen recognition, inhibitory chimeric antigen receptors, and so forth.

In some embodiments, CD30 fusion proteins are utilized in which the fusion is of the CD30 extracellular domain with another protein fragment, including a fragment that is at least part of a cytoplasmic region of a membrane bound protein. As one example, the CD30 extracellular domain and transmembrane domain (PVLFWVILVLVVVVGSSAFLL; SEQ ID NO:51) were fused with B-cell maturation antigen (BCMA) cytoplasmic tail (which is underlined). An amino acid sequence of a representative CD30-BCMA fusion protein is as follows:

(SEQ ID NO: 47)
MRVLLAALGLLFLGALRAFPQDRPFEDTCHGNPSHYYDKAVRRCC
YRCPMGLFPTQQCPQRPTDCRKQCEPDYYLDEADRCTACVTCSRD
DLVEKTPCAWNSSRVCECRPGMFCSTSAVNSCARCFFHSVCPAGM
IVKFPGTAQKNTVCEPASPGVSPACASPENCKEPSSGTIPQAKPT
PVSPATSSASTMPVRGGTRLAQEAASKLTRAPDSPSSVGRPSSDP
GLSPTQPCPEGSGDCRKQCEPDYYLDEAGRCTACVSCSRDDLVEK
TPCAWNSSRTCECRPGMICATSATNSCARCVPYPICAAETVTKPQ
DMAEKDTTFEAPPLGTQPDCNPTPENGEAPASTSPTQSLLVDSQA
SKTLPIPTSAPVALSSTGKPVLDAGPVLFWVILVLVVVVGSSAFL
LCHRKINSEPLKDEFKNTGSGLLGMANIDLEKSRTGDEIILPRGL
EYTVEECTCEDCIKSKPKVDSDHCFPLPAMEEGATILVTTKTNDY
CKSLPAALSATEIEKSISAR

The DNA sequence of a representative CD30-BCMA fusion protein is as follows:

ATGCGAGTCCTCCTGGCCGCGTTGGGGCTCTTGTTCCTTGGGGCACTTC GAGCCTTTCCACAGGATCGACCTTTTGAAGATACTTGTCACGGAAACCCTTCTCA CTACTACGATAAAGCGGTCCGACGATGCTGCTACCGATGCCCTATGGGACTTTTC CCGACGCAGCAATGCCCACAGCGGCCTACGGACTGTAGAAAGCAATGCGAGCCG GACTACTATCTGGACGAAGCAGATCGGTGTACTGCCTGCGTAACATGCTCCCGGG ATGACCTGGTGGAAAAGACCCCCTGCGCTTGGAACTCCAGTAGAGTATGCGAAT GCCGACCAGGGATGTTCTGCAGCACGAGCGCAGTTAACTCATGTGCCAGGTGTTT TTTCCATTCTGTATGTCCTGCCGGGATGATTGTTAAGTTCCCAGGTACAGCTCAA AAGAACACGGTATGCGAACCCGCTAGTCCGGGAGTTTCCCCGGCCTGCGCCAGC CCAGAGAATTGCAAGGAGCCGTCTAGCGGTACAATCCCACAAGCTAAGCCGACG CCGGTCAGCCCGGCGACTTCATCCGCCTCAACAATGCCCGTCCGGGGTGGGACA AGACTCGCGCAGGAAGCGGCTAGCAAGTTGACACGAGCCCCCGATTCCCCTTCA AGTGTAGGCAGACCTAGTTCCGACCCTGGCCTTAGCCCAACGCAACCCTGCCCAG AGGGATCCGGAGACTGTCGGAAACAATGCGAGCCCGACTATTACTTGGATGAAG CCGGTCGCTGTACCGCATGTGTGTCCTGCAGCCGCGATGACCTGGTCGAGAAAAC ACCATGTGCATGGAATAGCAGTCGCACCTGCGAGTGCCGACCGGGAATGATCTG TGCCACCTCAGCAACCAACTCTTGTGCCAGGTGCGTACCATATCCCATCTGCGCG GCTGAGACCGTAACAAAACCTCAAGACATGGCCGAAAAAGACACCACTTTCGAA GCGCCGCCTCTGGGCACTCAACCAGACTGCAATCCTACGCCAGAAAACGGGGAA GCACCCGCGTCAACCTCCCCCACACAATCTTTGCTCGTAGACTCTCAGGCTTCCA AAACACTGCCAATACCAACTTCCGCTCCGGTGGCTCTGAGCTCTACCGGCAAACC CGTGCTTGACGCCGGGCCAGTCCTGTTCTGGGTTATATTGGTGCTCGTAGTCGTA GTAGGCTCCAGCGCCTTTCTGCTCTGTCACCGGAAGATCAATTCCGAACCTTTGA AAGACGAGTTTAAGAACACCGGGAGTGGCCTCCTCGGAATGGCTAATATCGACT TGGAGAAGAGCCGCACTGGGGACGAAATCATTTTGCCTCGCGGGCTTGAATACA CAGTCGAAGAGTGCACGTGTGAAGACTGCATTAAATCAAAACCGAAGGTGGACA GCGATCATTGTTTCCCCTTGCCCGCTATGGAAGAAGGTGCAACTATCCTCGTAAC AACCAAAACTAACGATTATTGTAAAAGCCTCCCGGCGGCTCTCTCTGCGACGGA AATAGAAAAATCAATCTCTGCAAGG (SEQ ID NO:48), where the sequence that encodes the TM domain is underlined.

In some embodiments, wildtype CD30 is utilized because it is naturally present on cells, because it is a heterologous CD30 that was transduced into the cells, or because it became expressed on the cells as a results of transduction or transfection of one or more other genes into the cells. Wildtype full length CD30 is as follows:

Amino acid sequence:
(SEQ ID NO: 49)
MRVLLAALGLLFLGALRAFPQDRPFEDTCHGNPSHYYDKAVRRCC
YRCPMGLFPTQQCPQRPTDCRKQCEPDYYLDEADRCTACVTCSRD
DLVEKTPCAWNSSRVCECRPGMFCSTSAVNSCARCFFHSVCPAGM
IVKFPGTAQKNTVCEPASPGVSPACASPENCKEPSSGTIPQAKPT
PVSPATSSASTMPVRGGTRLAQEAASKLTRAPDSPSSVGRPSSDP
GLSPTQPCPEGSGDCRKQCEPDYYLDEAGRCTACVSCSRDDLVEK
TPCAWNSSRTCECRPGMICATSATNSCARCVPYPICAAETVTKPQ
DMAEKDTTFEAPPLGTQPDCNPTPENGEAPASTSPTQSLLVDSQA
SKTLPIPTSAPVALSSTGKPVLDAGPVLFWVILVLVVVVGSSAFL
LCHRRACRKRIRQKLHLCYPVQTSQPKLELVDSRPRRSSTQLRSG
ASVTEPVAEERGLMSQPLMETCHSVGAAYLESLPLQDASPAGGPS
SPRDLPEPRVSTEHTNNKIEKIYIMKADTVIVGTVKAELPEGRGL
AGPAEPELEEELEADHTPHYPEQETEPPLGSCSDVMLSVEEEGKE
DPLPTAASGK
DNA sequence:
(SEQ ID NO: 50)
atgcgcgtcctcctcgccgcgctgggactgctgttcctgggggcg
ctacgagccttcccacaggatcgacccttcgaggacacctgtcat
ggaaaccccagccactactatgacaaggctgtcaggaggtgctgt
taccgctgccccatggggctgttcccgacacagcagtgcccacag
aggcctactgactgcaggaagcagtgtgagcctgactactacctg
gatgaggccgaccgctgtacagcctgcgtgacttgttctcgagac
gacctcgtggagaagacgccgtgtgcatggaactcctcccgtgtc
tgcgaatgtcgacccggcatgttctgttccacgtctgccgtcaac
tcctgtgcccgctgcttcttccattctgtctgtccggcagggatg
attgtcaagttcccaggcacggcgcagaagaacacggtctgtgag
ccggcttccccaggggtcagccctgcctgtgccagcccagagaac
tgcaaggaaccctccagtggcaccatcccccaggccaagcccacc
ccggtgtccccagcaacctccagtgccagcaccatgcctgtaaga
gggggcaccegcctcgcccaggaagctgcttctaaactgacgagg
gctcccgactctccctcctctgtgggaaggcctagttcagatcca
ggtctgtccccaacacagccatgcccagaggggtctggtgattgc
agaaagcagtgtgagcccgactactacctggacgaggccggccgc
tgcacggcctgcgtgagctgttctcgagatgaccttgtggagaag
acgccatgtgcatggaactcctcccgcacctgcgaatgtcgacct
ggcatgatctgtgccacatcagccaccaactcctgtgcccgctgt
gtcccctacccaatctgtgcagcagagacggtcaccaagccccag
gatatggctgagaaggacaccacctttgaggcgccacccctgggg
acccagccggactgcaaccccaccccagagaatggcgaggcgcct
gccagcaccagccccactcagagcttgctggtggactcccaggcc
agtaagacgctgcccatcccaaccagegctcccgtcgctctctcc
tccacggggaagcccgttctggatgcagggccagtgctcttctgg
gtgatcctggtgttggttgtggtggtcggctccagegccttcctc
ctgtgccaccggagggcctgcaggaagcgaatteggcagaagctc
cacctgtgctacceggtccagacctcccagcccaagctagagctt
gtggattccagacccaggaggagctcaacgcagctgaggagtggt
gcgtcggtgacagaacccgtcgcggaagagcgagggttaatgagc
cagccactgatggagacctgccacagcgtgggggcagcctacctg
gagagcctgccgctgcaggatgccagcccggccgggggccccteg
tcccccagggaccttcctgagccccgggtgtccacggagcacacc
aataacaagattgagaaaatctacatcatgaaggctgacaccgtg
atcgtggggaccgtgaaggctgagctgccggagggccggggcctg
gggggccagcagagcccgagttggaggaggagctggaggcggacc
ataccccccactaccccgagcaggagacagaaccgcctctgggca
gctgcagcgatgtcatgctctcagtggaagaggaagggaaagaag
accccttgcccacagctgcctctggaaagtga

Other specific fusion protein combinations are contemplated, including (1) CD30 extracellular antigen with trop-2 intracellular domain, which may or may not comprise CD30 transmembrane domain; (2) CD30 extracellular antigen with CD317 intracellular domain, which may or may not comprise CD30 transmembrane domain; (3) CD30 extracellular antigen with CD3gamma intracellular domain, which may or may not comprise CD30 transmembrane domain; (3) CD30 extracellular antigen with CD4 intracellular domain, which may or may not comprise CD30 transmembrane domain; or (4) CD30 extracellular antigen with CD79b intracellular domain, which may or may not comprise CD30 transmembrane domain. In embodiments wherein the CD30 transmembrane is not used, the transmembrane domain of one of the following may be utilized: the alpha, beta or zeta chain of the T-cell receptor, CD28, CD3 zeta, CD3 epsilon, CD3 gamma, CD3 delta, CD45, CD4, CD5, CD8, CD9, CD 16, CD22, CD33, CD37, CD64, CD80, CD86, CD134, CD137, CD154, ICOS/CD278, GITR/CD357, NKG2D, or DAP molecules, such as DAP10 or DAP12.

### XI. Methods Related to CD30-Positive Cells

The disclosure concerns CD30-positive cells and uses thereof, including for either natural or recombinant CD30, including membrane-bound CD30 protein that may or may not be chimeric with one or more other protein fragments. In specific embodiments, the CD30 is utilized as follows: (1) transduction and selection marker to enrich therapeutic cellular products during manufacturing; (2) transduction marker for monitoring infused cellular products to assess expansion, phenotype, function, trafficking, and persistence in vivo; (3) safety switch to eliminate infused therapeutic cells as needed using monoclonal antibodies, antibody-drug conjugates, or other approaches.

In specific cases, the disclosure encompasses methods in which CD30 is used as a transduction marker. For example, a cell that may or may not express CD30 is transfected or transduced to express a protein having at least the CD30 extracellular domain, and candidate cells for successful transfection or transduction are assayed for the presence of the CD30 extracellular domain, such as using one or more agents that bind to at least part of the extracellular domain. In some cases, the one or more agents are labeled such that this interaction can be detected, including detection with light, fluorescence, color, radioactivity, and so forth. In other cases, a cell that may or may not express CD30 is transfected or transduced to express one or more proteins that are not CD30, but following the respective transfection or transduction the cells express CD30 as a direct or indirect result of the transfection or transduction.

In some embodiments, the CD30 protein is employed as a selection marker. That is, a population of cells having at least a subset that are suspected of being CD30-positive are subject to methods that allow for selection of the CD30-positive cells, such as using a substrate that comprises CD30 binding agents on the surface. The population may be exposed under sufficient conditions to allow for those CD30-positive cells in the population to bind the agent, thereby excluding those cells that are not CD30 positive. The cells may then be released from the binding agents, such as following appropriate washings. In specific embodiments, the CD30 used as a selection marker is not the endogenous CD30 but instead is expressed by the cells because of the hand of man, such as following transfection or transduction with a heterologous CD30, a CD30 fusion protein, or with one or more heterologous genes that are not CD30 but that result in its upregulation.

Methods of the disclosure also utilize CD30 or CD30-associated derivative proteins (e.g., fusion proteins) for monitoring cellular products. The monitoring may be in vitro or in vivo, and the monitoring may include assaying for expansion of the cells, particular functions associated with the cells, and so forth. In some cases, the monitoring is in vivo and follows administration of the CD30-positive cells into a recipient individual, including administration by infusion. In specific cases, the CD30-positive infused cellular products allow assessment of a variety of aspects of the infused cellular therapy in vivo, including expansion, phenotype, function, trafficking, and persistence. In some embodiments, the monitoring of the CD30-positive cells provides information of a risk of the cellular therapy to become toxic to the individual. For example, CAR T cells may show excessive proliferation after infusion and can cause toxicity.

In particular embodiments, CD30 or CD30-associated derivative proteins (e.g., fusion proteins) on the cells are utilized as a safety switch to inhibit activity of the CD30-positive cells of the cellular therapy in the event that the cells are no longer needed or that the individual is exhibiting one or more signs that the cellular therapy is toxic to the individual or has become toxic to the individual over a specific period of time of any duration. The present methods and compositions are useful to reduce or prevent one or more adverse events, such as cytokine release syndrome, neurotoxicity, immune effector cell-associated neurotoxicity syndrome, anaphylaxis/allergy, host rejection, including at least GVHD, on-target on-tumor toxicity, and/or on-target/off tumor toxicities (depletion of normal cells) or is considered at risk for having the one or more symptoms, including imminently. The use of the suicide gene or safety switch may be part of a planned protocol for a therapy or may be used only upon a recognized need for its use. In some cases, the safety switch delays onset of toxicity and/or reduces severity of toxicity, whereas in other cases it prevents toxicity or fully inhibits toxicity.

After infusion into individuals in need of the therapeutic cells, the membrane-bound CD30 proteins may be used to monitor the fate of the adoptively transferred T cells, including by flow cytometry, PCR, or other laboratory methods, such as next generation sequencing or using clinical tests such as imaging studies. In situations where the therapy results in adverse events such as cytokine release syndrome, GVHD, tumorigenesis, etc., an antibody or antibody-drug conjugate or other approaches may be utilized to eliminate the infused cells in vivo.

The disclosure provides membrane-bound CD30 proteins or fusion proteins that could be used alone or in combination as transduction and selection markers and/or as elimination safety switches. In some cases, the cells express the CD30 or CD30 fusion protein that are used as a safety switch but not as a transduction or selection marker, whereas in other cases they are used as a transduction or selection marker but not a safety switch.

Methods of the disclosure also encompass methods of inhibiting activity of cells by targeting a surface protein on the cells that comprises part or all of an extracellular domain of CD30. The activity that is inhibited may be of any kind, but in specific embodiments the inhibiting of activity is defined as inducing apoptosis for the cells. Upon exposure of the cells to an effective amount of one or more agents that bind CD30 may result in the proliferation of the cells being inhibited or delayed, or the cells may be killed. The agent(s) that binds CD30 may control survival of the cells in any method disclosed herein.

### XII. Combination Therapies

In certain embodiments, the compositions and methods of the present embodiments involve a cancer therapy that is additional to the compositions comprising therapeutic cells. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, hormone therapy, or a combination of the foregoing. The additional therapy may be in the form of adjuvant or neoadjuvant therapy.

In some embodiments, the additional therapy is the administration of small molecule enzymatic inhibitor(s) or anti-metastatic agent(s). In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PBK/AKT/mTOR pathway, HSP90 inhibitor, tubulin inhibitor, apoptosis inhibitor, and/or chemopreventative agent(s). The additional therapy may be one or more of the chemotherapeutic agents known in the art.

An immune cell therapy (in addition to the cell therapy of the disclosure) may be administered before, during, after, or in various combinations relative to an additional cancer therapy, such as immune checkpoint therapy. The administrations may be in intervals ranging from concurrently to minutes to days to weeks. In embodiments where the immune cell therapy is provided to a patient separately from the composition(s) of the disclosure, one would generally ensure that a significant period of time did not expire between the time of each delivery, such that the two compounds would still be able to exert an advantageously combined effect on the patient. In such instances, it is contemplated that one may provide a patient with the immunotherapy therapy and the disclosed compositions within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

Administration of any compound or cell therapy of the present embodiments to a patient will follow general protocols for the administration of such compounds, taking into account the toxicity, if any, of the agents. Therefore, in some embodiments there is a step of monitoring toxicity that is attributable to combination therapy.

### A. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclophosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammalI and calicheamicin omegaI1); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabien, navelbine, farnesyl-protein tansferase inhibitors, transplatinum, and pharmaceutically acceptable salts, acids, or derivatives of any of the above.

### B. Radiotherapy

Other factors that cause DNA damage and have been used extensively include what are commonly known as 7-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

### C. Immunotherapy

The skilled artisan will understand that additional immunotherapies (outside of the disclosed cell therapy) may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells other than those having knockdown or knockout of TGF-beta R2.

Antibody-drug conjugates have emerged as a breakthrough approach to the development of cancer therapeutics. Antibody-drug conjugates (ADCs) comprise monoclonal antibodies (MAbs) that are covalently linked to cell-killing drugs. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. The approval of two ADC drugs, ADCETRIS® (brentuximab vedotin) in 2011 and KADCYLA® (trastuzumab emtansine or T-DM1) in 2013 by FDA validated the approach. There are currently more than 30 ADC drug candidates in various stages of clinical trials for cancer treatment (Leal et al., 2014). As antibody engineering and linker-payload optimization are becoming more and more mature, the discovery and development of new ADCs are increasingly dependent on the identification and validation of new targets that are suitable to this approach and the generation of targeting MAbs. Two criteria for ADC targets are upregulated/high levels of expression in tumor cells and robust internalization.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons of any kind, IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

### D. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### E. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### XIII. Vectors

In particular embodiments, cells encompassed herein harbor one or more vectors that may express any polypeptide encompassed herein. The different proteins may be delivered to a recipient cell by any suitable vector, including by a viral vector or by a non-viral vector. Examples of viral vectors include at least retroviral, lentiviral, adenoviral, or adeno-associated viral vectors. Examples of non-viral vectors include at least plasmids, transposons, lipids, nanoparticles, liposomes, combinations thereof, and so forth.

In cases wherein the cell is transduced with a vector encoding a chimeric polypeptide, e.g., and also requires transduction of another gene or genes into the cell, such as a heterologous protein, they may or may not be comprised on or with the same vector. In some cases, the chimeric polypeptide, heterologous protein(s), etc., are expressed from the same vector molecule, such as the same viral vector molecule. In such cases, the expression of the polypeptides may or may not be regulated by the same regulatory element(s). When the polypeptides are on the same vector, they may or may not be expressed as separate polypeptides. In cases wherein they are expressed as separate polypeptides, they may be separated on the vector by a 2A element or IRES element (or both kinds may be used on the same vector once or more than once), for example.

One of skill in the art would be well-equipped to construct a vector through standard recombinant techniques (see, for example, Sambrook et al., 2001 and Ausubel et al., 1996, both incorporated herein by reference) for the expression of the antigen receptors of the present disclosure.

### A. Regulatory Elements

Expression cassettes included in vectors useful in the present disclosure in particular contain (in a 5′-to-3′ direction) a eukaryotic transcriptional promoter operably linked to a protein-coding sequence, splice signals including intervening sequences, and a transcriptional termination/polyadenylation sequence. The promoters and enhancers that control the transcription of protein encoding genes in eukaryotic cells may be comprised of multiple genetic elements. The cellular machinery is able to gather and integrate the regulatory information conveyed by each element, allowing different genes to evolve distinct, often complex patterns of transcriptional regulation. A promoter used in the context of the present disclosure includes constitutive, inducible, and tissue-specific promoters, for example. In cases wherein the vector is utilized for the generation of cancer therapy, a promoter may be effective under conditions of hypoxia.

### B. Promoter/Enhancers

The expression constructs provided herein comprise a promoter to drive expression of any polypeptide(s). A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, for example, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the β-lactamase (penicillinase), lactose and tryptophan (trp-) promoter systems. In addition to producing nucleic acid sequences of promoters and enhancers synthetically, sequences may be produced using recombinant cloning and/or nucleic acid amplification technology, including PCR, in connection with the compositions disclosed herein. Furthermore, it is contemplated that the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al., 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally, any promoter/enhancer combination (as per, for example, the Eukaryotic Promoter Data Base EPDB, through world wide web at epd.isb-sib.ch/) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

Non-limiting examples of promoters include early or late viral promoters, such as, SV40 early or late promoters, cytomegalovirus (CMV) immediate early promoters, Rous Sarcoma Virus (RSV) early promoters; eukaryotic cell promoters, such as, e. g., beta actin promoter, GADPH promoter, metallothionein promoter; and concatenated response element promoters, such as cyclic AMP response element promoters (cre), serum response element promoter (sre), phorbol ester promoter (TPA) and response element promoters (tre) near a minimal TATA box. It is also possible to use human growth hormone promoter sequences (e.g., the human growth hormone minimal promoter described at GENBANK®, accession no. X05244, nucleotide 283-341) or a mouse mammary tumor promoter (available from the ATCC®, Cat. No. ATCC 45007). In certain embodiments, the promoter is CMV IE, dectin-1, dectin-2, human CD11c, F4/80, SM22, RSV, SV40, Ad MLP, beta-actin, MHC class I or MHC class II promoter, however any other promoter that is useful to drive expression of the therapeutic gene is applicable to the practice of the present disclosure.

In certain aspects, methods of the disclosure also concern enhancer sequences, i.e., nucleic acid sequences that increase a promoter's activity and that have the potential to act in cis, and regardless of their orientation, even over relatively long distances (up to several kilobases away from the target promoter). However, enhancer function is not necessarily restricted to such long distances as they may also function in close proximity to a given promoter.

### C. Initiation Signals and Linked Expression

A specific initiation signal also may be used in the expression constructs provided in the present disclosure for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments, the use of internal ribosome entry sites (IRES) elements are used to create multigene, or polycistronic messages. IRES elements are able to bypass the ribosome scanning model of 5′ methylated Cap dependent translation and begin translation at internal sites. IRES elements from two members of the picornavirus family (polio and encephalomyocarditis) have been described, as well an IRES from a mammalian message. IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

As detailed elsewhere herein, certain 2A sequence elements could be used to create linked- or co-expression of genes in the constructs provided in the present disclosure. For example, cleavage sequences could be used to co-express genes by linking open reading frames to form a single cistron. An exemplary cleavage sequence is the equine rhinitis A virus (E2A) or the F2A (Foot-and-mouth disease virus 2A) or a “2A-like” sequence (e.g., Thosea asigna virus 2A; T2A) or porcine teschovirus-1 (P2A). In specific embodiments, in a single vector the multiple 2A sequences are non-identical, although in alternative embodiments the same vector utilizes two or more of the same 2A sequences. Examples of 2A sequences are provided in US 2011/0065779 which is incorporated by reference herein in its entirety.

### D. Origins of Replication

In order to propagate a vector in a host cell, it may contain one or more origins of replication sites (often termed “ori”), for example, a nucleic acid sequence corresponding to oriP of EBV as described above or a genetically engineered oriP with a similar or elevated function in programming, which is a specific nucleic acid sequence at which replication is initiated. Alternatively a replication origin of other extra-chromosomally replicating virus as described above or an autonomously replicating sequence (ARS) can be employed.

### E. Selection and Screenable Markers

In some embodiments, CD30-positive cells of the present disclosure may be identified in vitro or in vivo by including a marker in the expression vector. Such markers would confer an identifiable change to the cell permitting easy identification of cells containing the expression vector. Generally, a selection marker is one that confers a property that allows for selection. A positive selection marker is one in which the presence of the marker allows for its selection, while a negative selection marker is one in which its presence prevents its selection. An example of a positive selection marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selection markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes as negative selection markers such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selection and screenable markers are well known to one of skill in the art.

### XIV. Methods of Treatment

In various embodiments, expression constructs, nucleic acid sequences, vectors, host cells and so forth as contemplated herein and/or pharmaceutical compositions comprising the same are used for the prevention, treatment or amelioration of a cancerous disease, such as a tumorous disease. In particular embodiments, the pharmaceutical composition of the present disclosure may be particularly useful in preventing, ameliorating and/or treating cancer, for example. The individual may utilize the treatment method of the disclosure as an initial treatment or after (or with) another treatment, such as following HSCT, for example. The immunotherapy methods may be tailored to the need of an individual with cancer based on the type and/or stage of cancer, and in at least some cases the immunotherapy may be modified during the course of treatment for the individual.

In some embodiments, the present disclosure provides methods for immunotherapy comprising administering an effective amount of the cells produced by methods of the present disclosure. In one embodiment, a medical disease or disorder is treated by transfer of cell populations produced by methods herein and that elicit an immune response. In certain embodiments of the present disclosure, cancer is treated by transfer of a cell population produced by methods of the disclosure. Provided herein are methods for treating or delaying progression of cancer in an individual comprising administering to the individual an effective amount cell therapy. The present methods may be applied for the treatment of solid cancers or hematologic cancers.

Tumors for which the present treatment methods are useful include any malignant cell type, such as those found in a solid tumor or a hematological tumor. Exemplary solid tumors can include, but are not limited to, a tumor of an organ selected from the group consisting of acute myeloid leukemia, lymphoma, lung cancer, renal cancer, bladder cancer, melanoma, glioblastoma, breast cancer, head and neck cancer, mesothelioma, multiple myeloma, and pancreatic cancer.

In certain embodiments of the present disclosure, immune cells as encompassed herein are delivered to an individual in need thereof, such as an individual that has cancer. In some cases, the individual is provided with one or more doses of the immune cells. In cases where the individual is provided with two or more doses of the immune cells, the duration between the administrations should be sufficient to allow time for propagation in the individual, and in specific embodiments the duration between doses is 1, 2, 3, 4, 5, 6, 7, or more days.

In specific embodiments, the cells are provided to an individual in a therapeutically effective amount (in a range from 1 cell to 10¹⁰) that ameliorates at least one symptom related to cancer in the individual. A therapeutically effective amount may be from 1 to 10¹⁰, 10 to 10¹⁰, 10²-10¹⁰, 10³ to 10¹⁰, 10³ to 10⁹, 10³ to 10⁸, 10³ to 10⁷, 10³ to 10⁶, 10¹ to 10⁵, 10¹ to 10⁴, 10⁴ to 10¹⁰, 10⁴ to 10⁹, 10⁴ to 10⁸, 10⁴ to 10⁷, 10⁴ to 10⁶, 10⁴ to 10⁵, 10⁵ to 10¹⁰, 10⁵ to 10⁹, 10⁵ to 10⁸, 10⁵ to 10⁷, 10⁵ to 10⁶, 10⁶ to 10¹⁰, 10⁶ to 10⁹, 10⁶ to 10⁸, 10⁶ to 10⁷, 10⁷ to 10¹⁰, 10⁷ to 10⁹, 10⁷ to 10⁸, 10⁸ to 10¹⁰, 10⁸ to 10⁹, or 10⁹ to 10¹⁰ cells. In specific embodiments, an individual having a cancer is provided once or multiple times a therapeutically effective amount of particular therapeutic cells. In some embodiments, when an individual is at risk for deleterious effects with these cells as therapy, the concentration may be adjusted and/or there may be administration of one or more chimeric polypeptide agents that target an antigen on the cells. In specific embodiments, an individual having a cancer is provided once or multiple times a therapeutically effective amount of particular immune cells, and then when an individual is at risk for deleterious effects with the cells as therapy, the concentration may be adjusted and/or there may be administration of one or more chimeric polypeptide agents that target an antigen on the cells.

In certain embodiments, subsequent to administration, vectors may be stably integrated into the genome of the subject. In specific embodiments, viral vectors may be used that are specific for certain cells or tissues and persist in the cells, for example. Suitable pharmaceutical carriers and excipients are well known in the art. The compositions prepared according to the disclosure can be used for the prevention or treatment or delaying the above identified diseases.

Furthermore, the disclosure relates to a method for the prevention, treatment or amelioration of a tumorous disease comprising the step of administering to a subject in the need thereof an effective amount of CD30-positive cells as encompassed herein, a nucleic acid sequence, a vector, as contemplated herein and/or produced by a process as contemplated herein.

Possible indications for administration of the composition(s) of the exemplary cells are cancerous diseases, including tumorous diseases of any kind. Exemplary indications for administration of the composition(s) of CD30-positive cells are cancerous diseases, including any malignancies that express a particular antigen, including one to which a CAR is directed. The administration of the composition(s) of the disclosure is useful for all stages and types of cancer, including for minimal residual disease, early cancer, advanced cancer, and/or metastatic cancer and/or refractory cancer, for example.

Therapeutically effective amounts of the produced cells can be administered by a number of routes, including parenteral administration, for example, intravenous, intraperitoneal, intramuscular, intrasternal, intratumoral, intrathecal, intraventricular, through a reservoir, intraarticular injection, or infusion.

The therapeutically effective amount of the produced cells for use in adoptive cell therapy is that amount that achieves a desired effect in a subject being treated. For instance, this can be the amount of immune cells necessary to inhibit advancement, or to cause regression of cancer.

The produced cell population can be administered in treatment regimens consistent with the disease, for example a single or a few doses over one to several days to ameliorate a disease state or periodic doses over an extended time to inhibit disease progression and prevent disease recurrence. The precise dose to be employed in the formulation will also depend on the route of administration, and the seriousness of the disease or disorder, and should be decided according to the judgment of the practitioner and each patient's circumstances. The therapeutically effective amount of cells will be dependent on the subject being treated, the severity and type of the affliction, and the manner of administration. In some embodiments, doses that could be used in the treatment of human subjects range from at least 1×10³, at least 1×10⁴, 3.8×10⁴, at least 3.8×10⁵, at least 3.8×10⁶, at least 3.8×10⁷, at least 3.8×10⁸, at least 3.8×10⁹, or at least 3.8×10¹⁰ T cells/m². In a certain embodiment, the dose used in the treatment of human subjects ranges from about 3.8×10⁹ to about 3.8×10¹⁰ T cells/m². In additional embodiments, a therapeutically effective amount of T cells can vary from about 5×10⁶ cells per kg body weight to about 7.5×10⁸ cells per kg body weight, such as about 2×10⁷ cells to about 5×10⁸ cells per kg body weight, or about 5×10⁷ cells to about 2×10⁸ cells per kg body weight. The exact amount of T cells is readily determined by one of skill in the art based on the age, weight, sex, and physiological condition of the subject. Effective doses can be extrapolated from dose-response curves derived from in vitro or animal model test systems.

The disclosure further encompasses co-administration protocols with other compounds, e.g. bispecific antibody constructs, targeted toxins or other compounds, which act via immune cells. The clinical regimen for co-administration of the inventive compound(s) may encompass co-administration at the same time, before or after the administration of the other component. Particular combination therapies include chemotherapy, radiation, surgery, hormone therapy, or other types of immunotherapy.

Embodiments relate to a kit comprising cells as defined herein, constructs as defined herein, a nucleic acid sequence as defined herein, a vector as defined herein and/or a host as defined herein. It is also contemplated that the kit of this disclosure comprises a pharmaceutical composition as described herein above, either alone or in combination with further medicaments to be administered to an individual in need of medical treatment or intervention.

### XV. Kits of the Disclosure

Any of the compositions described herein may be comprised in a kit. In a non-limiting example, the kit comprises CD30 molecules, cells encompassing same, vectors encoding particular CD30 proteins or CD30 fusion proteins, vectors encoding heterologous protein(s) and/or reagents to generate same, and any of these may be comprised in suitable container means in a kit of the present disclosure. Kits may comprise immune cells, vectors, expression construct polynucleotides for insertion into a vector (whether viral or not), and so forth. Primers for amplification of any polynucleotide may be included. In some cases, the kit comprises cryopreserved cells, including CD30-positive cells. Any reagents for transfection or transduction of the cells may be included.

The compositions of the kits may be packaged either in aqueous media or in lyophilized form. The container means of the kits will generally include at least one vial, test tube, flask, bottle, syringe or other container means, into which one or more components may be placed, and preferably, suitably aliquoted. Where there are more than one component in the kit, the kit also may generally contain a second, third or other additional container into which the additional components may be separately placed. However, various combinations of components may be comprised in a vial. The kits of the present disclosure also will typically include a means for containing the molecules, cells encompassing same, and/or reagents to generate same in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly envisioned. The compositions may also be formulated into a syringeable composition. In which case, the container means may itself be a syringe, pipette, and/or other such like apparatus, from which the formulation may be applied to an infected area of the body, injected into an animal, and/or even applied to and/or mixed with the other components of the kit.

However, the components of the kit may be provided as dried powder(s). When reagents and/or components are provided as a dry powder, the powder can be reconstituted by the addition of a suitable solvent. It is envisioned that the solvent may also be provided in another container means.

Irrespective of the number and/or type of containers, the kits of the disclosure may also comprise, and/or be packaged with, an instrument for assisting with the injection/administration and/or placement of the ultimate composition within the body of an animal. Such an instrument may be a syringe, pipette, forceps, and/or any such medically approved delivery vehicle. In some embodiments, reagents or apparatuses or containers are included in the kit for ex vivo use.

### XVI. Examples

The following examples are included to demonstrate certain embodiments of the invention. It should be appreciated by those of skill in the art that the techniques disclosed in the examples which follow represent techniques discovered by the inventor to function well in the practice of the invention, and thus can be considered to constitute certain modes for its practice. However, those of skill in the art should, in light of the present disclosure, appreciate that many changes can be made in the specific embodiments which are disclosed and still obtain a like or similar result without departing from the spirit and scope of the invention.

### Example 1—BCMA Extracellular Domain Expression on 293T Cells

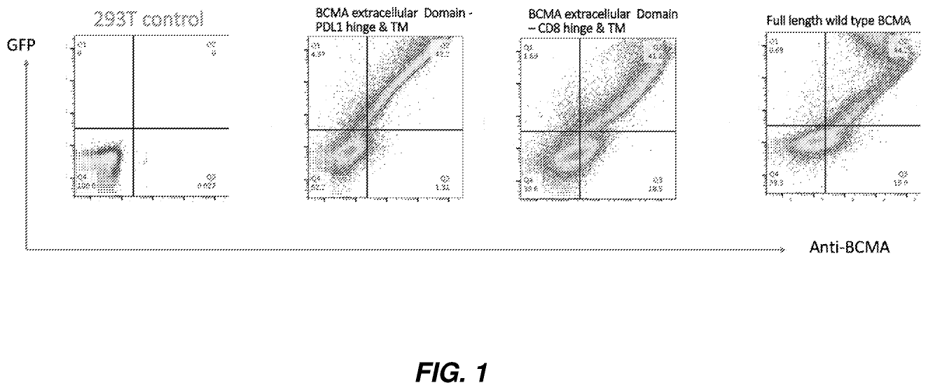

293T cells were transfected with plasmids containing one of three different membrane bound receptors: BCMA extracellular domain fused to PD-L1 hinge and transmembrane domain, BCMA extracellular domain fused to CD8a hinge and transmembrane domain, and full-length wild type BCMA. The polypeptides are described in Table 2 below. Each receptor was connected to enhanced GFP by a P2A peptide. One day after transfection, the cells were stained with an APC-conjugated anti-BCMA monoclonal antibody (FIG. 1).

TABLE 2

| | SEQ ID NO | SEQ ID NO |
|---|---|---|
| Polypeptide | (protein) | (DNA) |
| BCMA extracellular domain - PDL1 | 1 | 2 |
| hinge & TM | | |
| BCMA extracellular domain - CD8 | 3 | 4 |
| hinge & TM | | |
| Full length wild type BCMA | 5 | 6 |

### Example 2—Expression of BCMA Extracellular Domain with or without Internalization Motif in Therapeutic T Cells

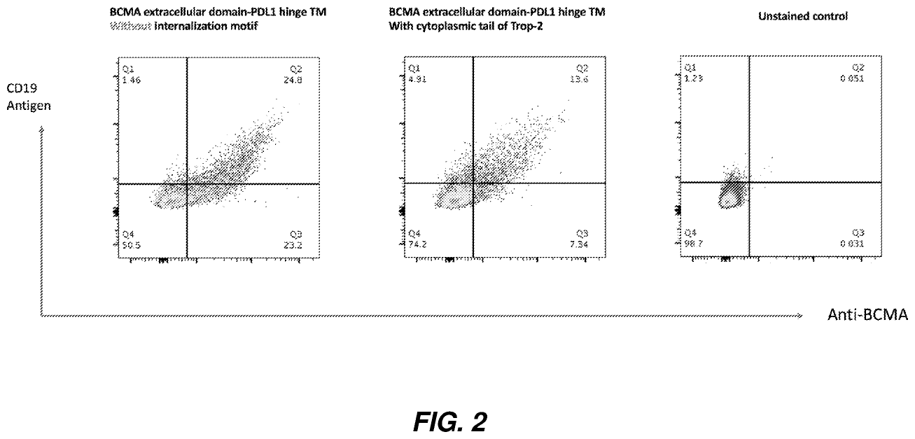

Primary T cells transduced with BCL6 and BCL2L1 were transduced with a lentiviral vector co-expressing a CD19 CAR and one of two different BCMA fusion constructs: BCMA extracellular domain with PD-L1 hinge and transmembrane domain but no functional intracellular domain, (shown on the left panel of FIG. 2) or BCMA extracellular domain fused with PD-L1 hinge and transmembrane domain, and cytoplasmic domain from Trop-2 (tBCMA; shown on the middle panel of FIG. 2). The polypeptides are described in Table 3 below.

The cells were stained with APC-conjugated anti-BCMA antibody and the recombinant FITC-labeled CD19-Fc protein (FIG. 2). The results show that the chimeric BCMA protein expression is consistent with CD19 CAR expression indicating that the chimeric BCMA proteins may be used as transduction markers for therapeutic T cells.

TABLE 3

| | SEQ ID NO | SEQ ID NO |
|---|---|---|
| Polypeptide | (protein) | (DNA) |
| BCMA extracellular domain - PDL1 | 1 | 2 |
| hinge TM | | |
| Without internalization motif | | |
| BCMA extracellular domain - PDL1 | 38 | 39 |
| hinge TM | | |
| With cytoplasmic tail of Trop-2 | | |

### Example 3—BCMA Fusion Protein Transduced into Therapeutic T Cells can be Used to Enrich T Cells Using Anti-BCMA Beads

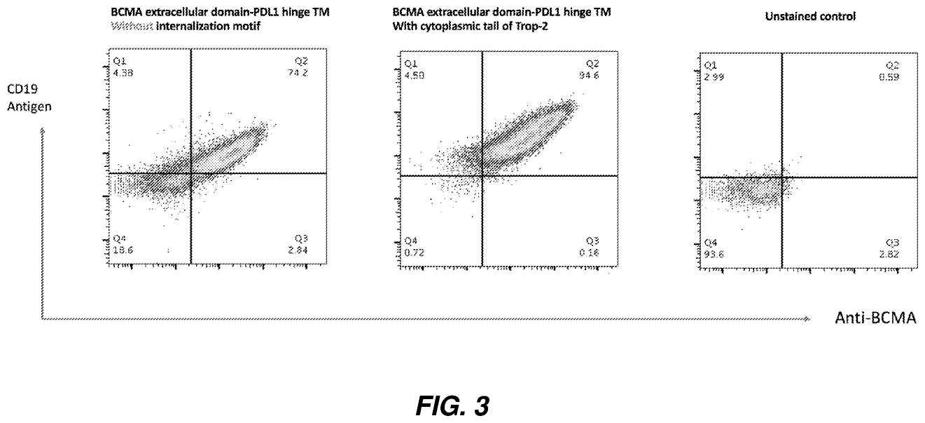

Primary T cells transduced with BCL6 and BCL2L1 were transduced as described in Example 2, stained with APC-conjugated anti-BCMA antibody, and then enriched using anti-APC magnetic beads. After 2 weeks of expansion, the cells were stained with APC-conjugated anti-BCMA antibody and recombinant FITC-labeled CD19-Fc protein to check for purity of transduced cells (FIG. 3). The results show that BCMA and anti-CD19 CAR double positive population were markedly enriched by magnetic bead isolation.

### Example 4—Fusion Protein Containing Trop2 Cytoplasmic Domain as a Safety Switch to Eliminate T Cells

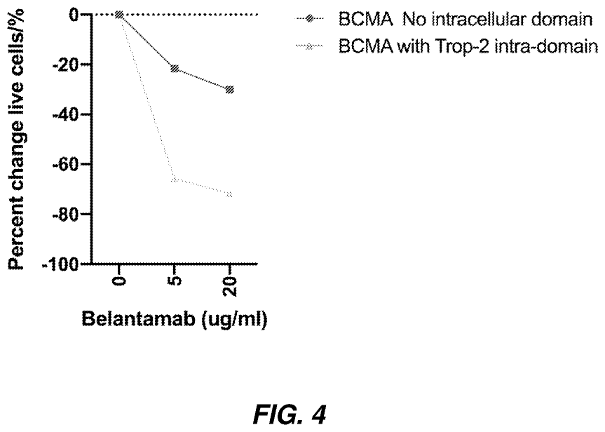

Primary T cells transduced with BCL6 and BCL2L1 were transduced with BCMA fusion proteins and enriched as described in Example 3 were cultured in the absence or presence of belantamab mafodotin (an antibody-drug conjugate targeting BCMA) at a concentration of 0 μg/mL, 5 μg/mL, or 20 μg/mL. On day 2, the live cells were counted by flow cytometry using counting beads and percent change in live cells with belantamab mafodotin compared to no drug was calculated (FIG. 4). The results show that the therapeutic T cells expressing BCMA with Trop-2 intracellular domain (tBCMA) were highly sensitive to cytotoxic activity of belantamab mafodotin.

### Example 5—Expression of BCMA Extracellular Domain with or without a Cytoplasmic Tail on Jurkat Cells

Jurkat cells were transduced with a lentiviral vector expressing BCMA extracellular domain alone, BCMA extracellular domain fused with cytoplasmic domain from CD317, or BCMA extracellular domain fused with cytoplasmic domain from CD3γ. The polypeptides are described in Table 4 below.

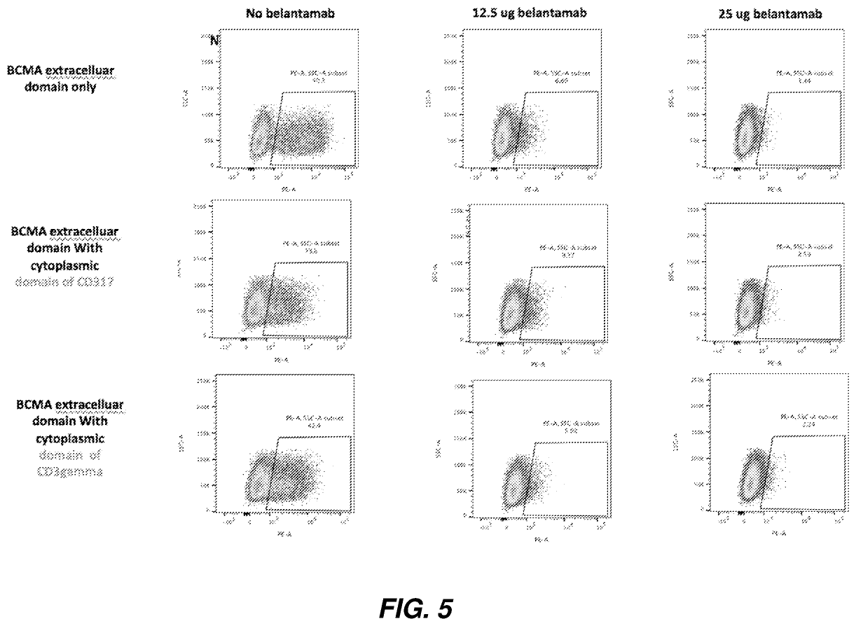

The cells were cultured in the absence or presence of belantamab mafodotin at a concentration of 25 μg/mL or 12.5 μg/mL. After 4 days, the culture medium was replaced with new medium without belantamab. After culturing for an additional 7 days, the percentage of BCMA positive cells was determined by flow cytometry (FIG. 5). The results show that the percentage of BCMA positive cells decreased in a dose dependent manner indicating specific killing by belantamab of BCMA positive cells.

TABLE 4

| | SEQ ID NO | SEQ ID NO |
|---|---|---|
| Polypeptide | (protein) | (DNA) |
| BCMA extracellular domain only | 41 | 42 |
| BCMA extracellular domain with | 5 | 6 |
| cytoplasmic | | |
| domain of CD317 | | |
| BCMA extracellular domain with | 7 | 8 |
| cytoplasmic | | |
| domain of CD3γ | | |

### Example 6—CD30 as a Safety Switch

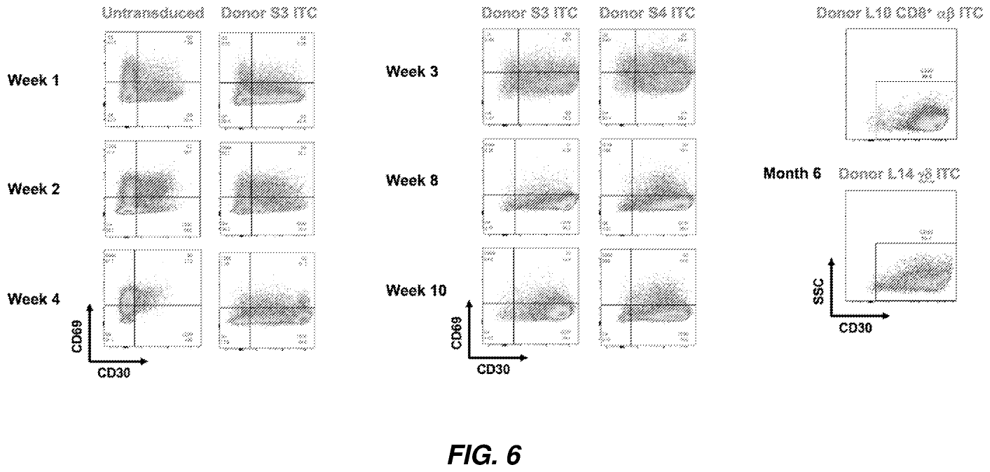

CD30 expression on T cells can be induced or maintained on T cells by over expression of BCL6 and BCL2L1 genes. BCL6 and BCL2L1 genes were transfected into primary T cells. Expression of CD30 and CD69 were measured by flow cytometry (FIG. 6). CD30 was constitutively expressed on both αβ and γδ T cells (>99% of the cells). In contrast, ˜15% of primary T cells cultured in vitro for 4 weeks expressed CD30. Thus, CD30 may be used as a selection marker during manufacturing, as transduction marker for monitoring in patients post-infusion, and as safety switch for elimination of the therapeutic cells in case of serious adverse events

### Example 7—Use of CD30 as a Safety Switch in Therapeutic T Cells Expressing CD30

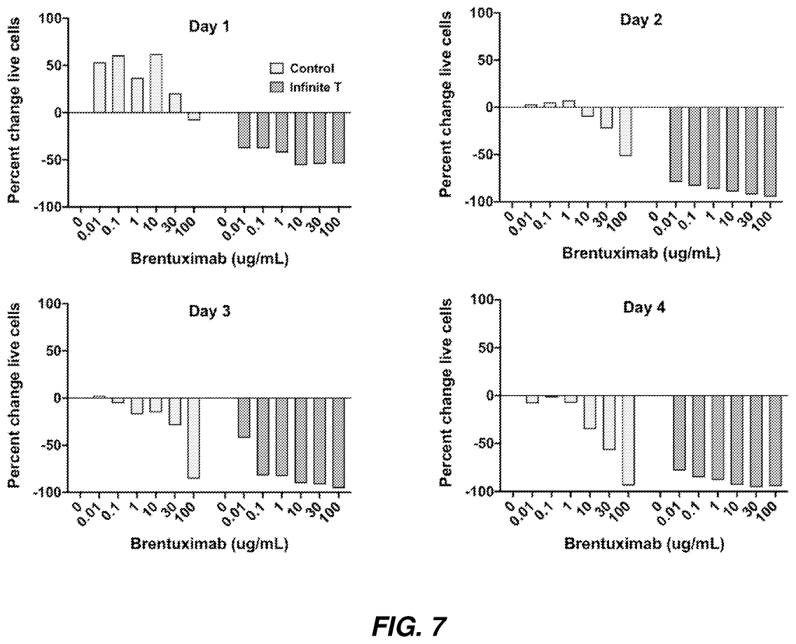

Primary T cells transduced with BCL6 and BCL2L1 expressing CD30 were cultured in the absence or presence of increasing concentrations of brentuximab vedotin. Raji Burkitt lymphoma tumor cells that do not express CD30 were used as controls. Cells were harvested, stained with live/dead stain, and absolute number of live cells was determined by flow cytometry using counting beads on days 1, 2, 3, and 4. Percent change in live cell number was determined as compared to cells cultured in the absence of brentuximab and shown in the graphs of FIG. 7. Data suggest efficient killing (up to 95%) of the CD30-expressing T cells compared to control Raji cells. Non-specific cytotoxicity was observed at higher concentrations and with longer incubation.

### Example 8—CD30 Extracellular Domain Fused with the BCMA Cytoplasmic Tail can be Expressed on 293T Cells

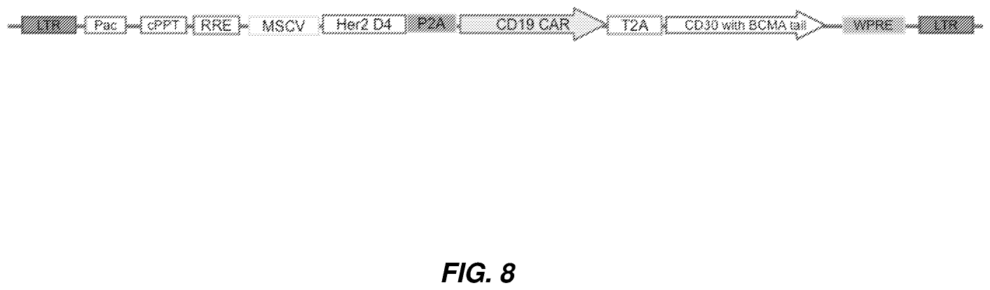

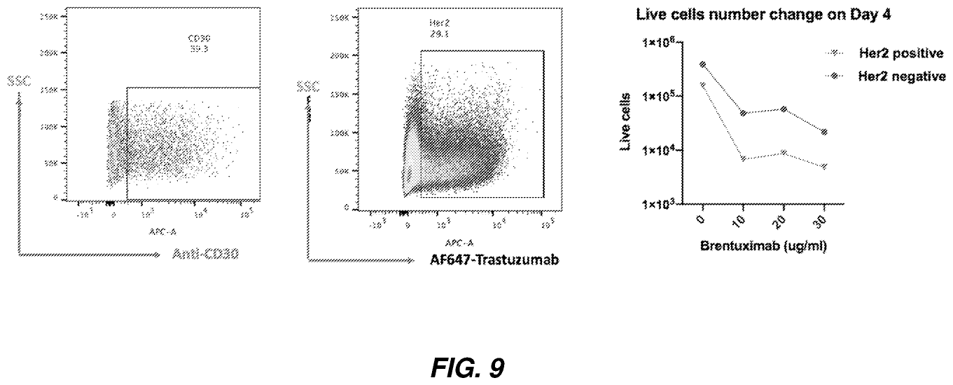

293T cells were transduced with the lentiviral vector expressing a CD19 CAR and both Her2 domain 4 and CD30 extracellular domain fused with BCMA cytoplasmic tail (diagram shown in FIG. 8). The polypeptides are described in Table 5 below. Transduced 293T were stained by both anti-CD30 and AF647 conjugated trastuzumab (FIG. 9). The transduced 293T cells were cultured in the presence of different concentrations of brentuximab for 4 days, the cell number change of both Her2 positive (also expected to be CD30 positive) and Her2 negative (also expected to be CD30 negative) 293T cells were counted using flow counting beads on day 4 (FIG. 9, right panel). Results showed that live cell count of CD30 positive (Her2 positive) 293T cells decreased significantly compared with controls.

TABLE 5

| | SEQ ID NO | SEQ ID NO |
|---|---|---|
| Polypeptide | (protein) | (DNA) |
| CD30 extracellular domain and TM domain | 9 | 10 |
| fused with BCMA cytoplasmic tail | | |
| Domain IV and Transmembrane domain of | 11 | 12 |
| HER2 | | |

### Example 9—Jurkat Cells Expressing Her2 Domain 4 can be Killed by CAR-T Cells

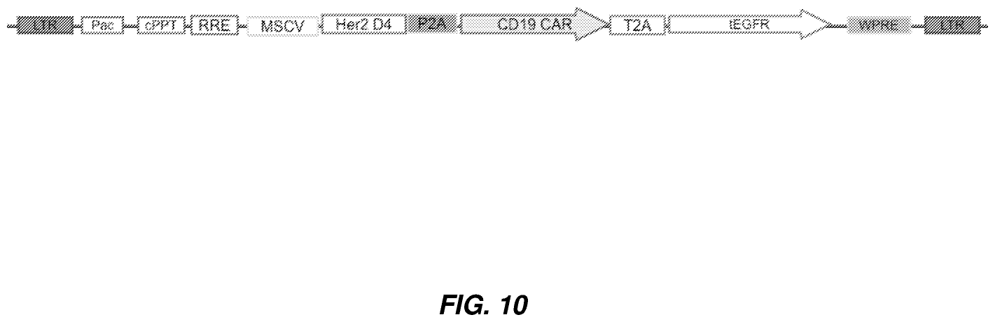

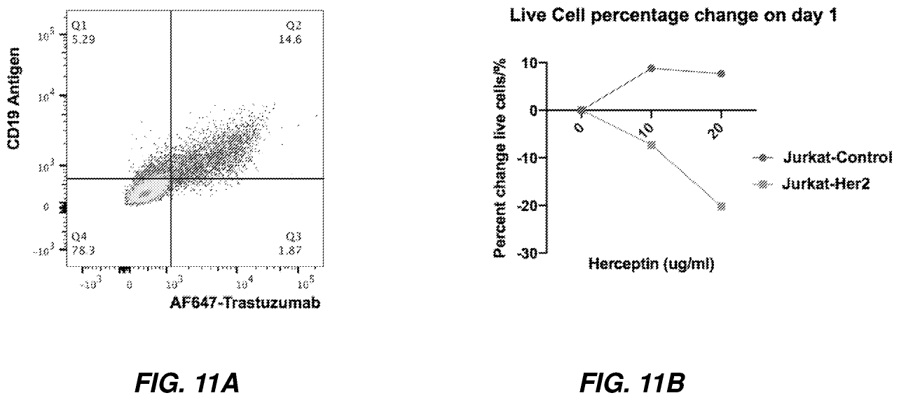

Jurkat T cells were transduced with a lentiviral vector expressing CD19 CAR and both Her2 and truncated EGFR (diagram shown in FIG. 10). The polypeptides are described in Table 6 below. The transduced cells were killed in the presence of trastuzumab and CD16-expressing CAR-T cells (FIGS. 11A-11B). FIG. 11A shows expression of CD19 CAR and Her2 domain 4 as detected by staining with CD19-Fc fusion protein and AF647-conjugated trastuzumab, respectively. FIG. 11B shows percentage change in the live Jurkat cells when cocultured with CD16-expressing CAR-T in the presence of trastuzumab.

TABLE 6

| | SEQ ID NO | SEQ ID NO |
|---|---|---|
| Polypeptide | (protein) | (DNA) |
| Domain IV and Transmembrane | 11 | 12 |
| domain of HER2 | | |
| Truncated EGFR (EGFR domain 3-partial | 15 | 16 |
| domain 4 and CD8 hinge and TM) | | |

### Example 10—Trop2 as a Safety Switch

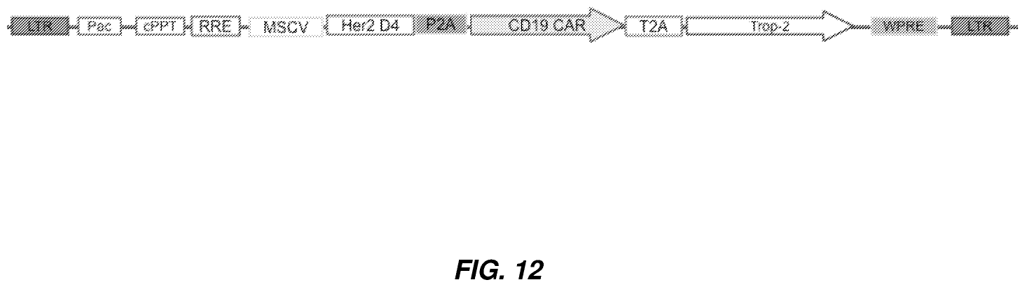

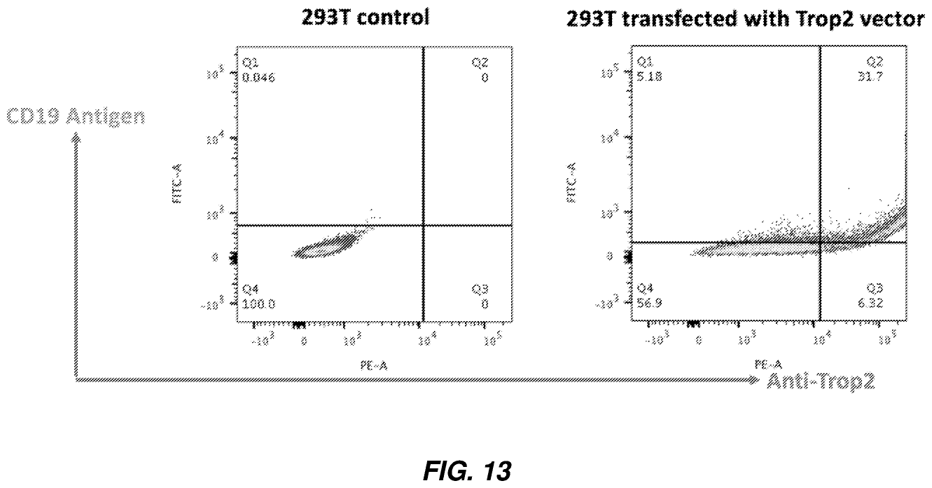

293T cells were transfected with lentiviral plasmid containing anti-CD19 CAR, Her2 domain IV and Trop2 gene (diagram shown in FIG. 12) using LIPOFECTAMINE™ 3000. The polypeptides are described in Table 7 below. Flow cytometry analysis was performed 24 hours after transfection (FIG. 13). Data shows co-expression of anti-CD19 CAR and Trop2 in transfected cells.

TABLE 7

| | SEQ ID NO | SEQ ID NO |
|---|---|---|
| Polypeptide | (protein) | (DNA) |
| Domain IV and Transmembrane | 11 | 12 |
| domain of HER2 | | |
| Trop2 | 13 | 14 |

### Example 11—Domain 3 and Partial Domain 4 of EGFR as Safety Switch

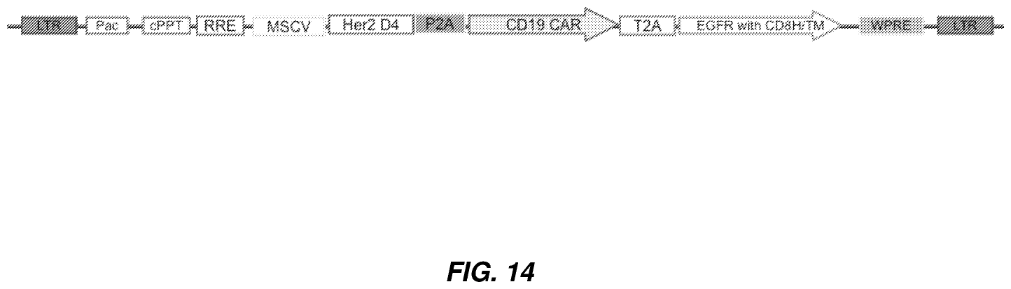

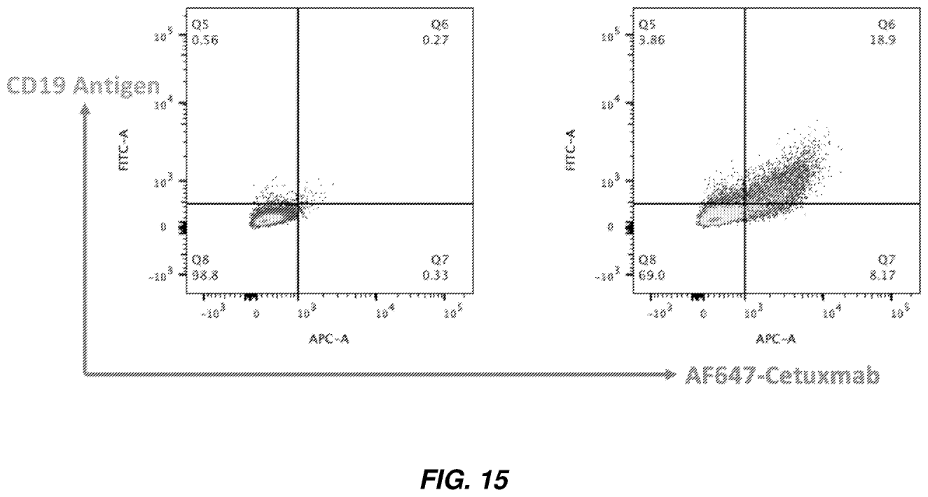

293T cells were transfected with lentiviral plasmid containing CD19 CAR and both Her2 and truncated EGFR (EGFR domain 3-partial domain 4 and CD8 hinge and TM; construct diagram shown in FIG. 14). Sequences for the truncated EGFR (EGFR domain 3-partial domain 4 and CD8 hinge and TM) are provided in Table 8 below. Data shows co-expression of anti-CD19 CAR and truncated EGFR in transfected cells detected by CD19-Fc fusion protein and AF647-conjugated cetuximab (FIG. 15, right panel). FIG. 15, left panel shows untransduced 293T cells.

TABLE 8

| | SEQ ID NO | SEQ ID NO |
|---|---|---|
| Polypeptide | (protein) | (DNA) |
| Truncated EGFR (EGFR domain 3-partial | 15 | 16 |
| domain 4 and CD8 hinge and TM) | | |

### Example 12—tBCMA Safety Switch has Both In Vitro and In Vivo Efficacy

Primary T cells transduced with BCL6 and BCL2L1+/−CD19 CAR and expressing truncated BCMA (tBCMA) were cultured in the absence or presence of increasing concentrations of belantamab mafodotin, an anti-BCMA antibody drug-conjugate. Raji Burkitt lymphoma tumor cells that do not express BCMA were used as controls. Cells were harvested, stained with live/dead stain, and absolute number of live cells was determined by flow cytometry using counting beads on day 2. Percent change in live cell number was determined as compared to cells cultured in the absence of brentuximab and shown in the graph. Data suggest efficient killing (up to 80%) of the BCMA-expressing T cells compared to control Raji cells. Non-specific cytotoxicity was observed at higher concentrations.

Primary T cells transduced with BCL6, BCL2L1, CD19 CAR, tBCMA, luciferase, and IL-15 were injected into NSG mice IV via tail vein on Day 0 (2×106 cells/mouse). Belantamab mafodotin was injected IV via tail vein at a dose of 2.5 mg/kg body weight in mice 3 and 4 on day 3 and in mice 1 and 2 on day 11. T cell expansion and persistence was monitored by bioluminescence imaging at the indicated time points. The data show that the T cells can be very efficiently eradicated with belantamab mafodotin when injected before (mice 3 and 4) or after robust expansion (mice 1 and 2) in vivo.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A chimeric polypeptide comprising:
(a) an extracellular domain from B-cell maturation antigen (BCMA);
(b) a hinge region from programmed cell death 1 ligand 1 (PDL1);
(c) a transmembrane domain; and
(d) an intracellular region.

2. The chimeric polypeptide of claim 1, wherein the extracellular domain comprises an amino acid sequence at least 95% identical to SEQ ID NO:19.

3. The chimeric polypeptide of claim 2, wherein the extracellular domain comprises SEQ ID NO:19.

4. The chimeric polypeptide of claim 3, wherein the extracellular domain consists of SEQ ID NO:19.

5. The chimeric polypeptide of any one of claims 1-4, wherein the hinge region comprises SEQ ID NO:23.

6. The chimeric polypeptide of claim 5, wherein the hinge region consists of SEQ ID NO:23.

7. The chimeric polypeptide of any one of claims 1-6, wherein the transmembrane domain is an alpha or beta chain of the T cell receptor or a transmembrane domain from CD28, CD3e, CD45, CD4, CD5, CD8, CD9, CD16, CD22, CD33, CD37, CD64, CD80, CD86, CD123, CD134, CD137 or CD154.

8. The chimeric polypeptide of any one of claims 1-7, wherein the transmembrane domain is a transmembrane domain from CD8α.

9. The chimeric polypeptide of any one of claims 1-8, wherein the transmembrane domain comprises an amino acid sequence at least 95% identical to SEQ ID NO:26.

10. The chimeric polypeptide of claim 9, wherein the transmembrane domain comprises SEQ ID NO:26.

11. The chimeric polypeptide of claim 10, wherein the transmembrane domain consists of SEQ ID NO:26.

12. The chimeric polypeptide of any one of claims 1-6, wherein the transmembrane domain is a transmembrane domain from PDL1.

13. The chimeric polypeptide of claim 12, wherein the transmembrane domain comprises an amino acid sequence at least 95% identical to SEQ ID NO:25.

14. The chimeric polypeptide of claim 13, wherein the transmembrane domain comprises SEQ ID NO:25.

15. The chimeric polypeptide of claim 14, wherein the transmembrane domain consists of SEQ ID NO:25.

16. The chimeric polypeptide of any one of claims 1-15, wherein the intracellular region comprises the sequence RLR (SEQ ID NO:29).

17. The chimeric polypeptide of claim 16, wherein the intracellular region consists of the sequence RLR (SEQ ID NO:29).

18. The chimeric polypeptide of any one of claims 1-11, wherein the intracellular region comprises SEQ ID NO:31.

19. The chimeric polypeptide of claim 18, wherein the intracellular region consists of SEQ ID NO:31.

20. The chimeric polypeptide of any one of claims 1-11, wherein the intracellular region comprises SEQ ID NO:32.

21. The chimeric polypeptide of claim 20, wherein the intracellular region consists of SEQ ID NO:32.

22. The chimeric polypeptide of any one of claims 1-11, wherein the intracellular region comprises an amino acid sequence at least 95% identical to SEQ ID NO:40.

23. The chimeric polypeptide of claim 22, wherein the intracellular region comprises SEQ ID NO:40.

24. The chimeric polypeptide of claim 23, wherein the intracellular region consists of SEQ ID NO:40.

25. The chimeric polypeptide of any one of claims 1-24, wherein the intracellular region comprises at most 25 amino acids.

26. The chimeric polypeptide of any one of claims 1-25, wherein the intracellular region comprises at most 20 amino acids.

27. The chimeric polypeptide of any one of claims 1-26, wherein the intracellular region comprises at most 10 amino acids.

28. The chimeric polypeptide of any one of claims 1-27, wherein the intracellular region comprises at most 6 amino acids.

29. The chimeric polypeptide of any one of claims 1-28, wherein the intracellular region comprises at most 3 amino acids.

30. The chimeric polypeptide of any one of claims 1-29, wherein the chimeric polypeptide is less than or equal to 100 amino acids in length.

31. The chimeric polypeptide of any one of claims 1-30, wherein the chimeric polypeptide does not comprise a signaling domain.

32. The chimeric polypeptide of any one of claims 1-31, wherein the chimeric polypeptide does not comprise an intracellular region from BCMA.

33. The chimeric polypeptide of any one of claims 1-32, wherein the chimeric polypeptide comprises an amino acid sequence at least 95% identical to SEQ ID NO:1.

34. The chimeric polypeptide of claim 33, wherein the chimeric polypeptide comprises an amino acid sequence at least 99% identical to SEQ ID NO:1.

35. The chimeric polypeptide of claim 34, wherein the chimeric polypeptide comprises SEQ ID NO:1.

36. A chimeric polypeptide comprising an amino acid sequence at least 95% identical to SEQ ID NO:1.

37. A chimeric polypeptide comprising SEQ ID NO:1.

38. A chimeric polypeptide consisting of SEQ ID NO:1.

39. The chimeric polypeptide of any one of claims 1-32, wherein the chimeric polypeptide comprises an amino acid sequence at least 95% identical to SEQ ID NO:38.

40. The chimeric polypeptide of claim 39, wherein the chimeric polypeptide comprises an amino acid sequence at least 99% identical to SEQ ID NO:38.

41. The chimeric polypeptide of claim 40, wherein the chimeric polypeptide comprises SEQ ID NO:38.

42. A chimeric polypeptide comprising an amino acid sequence at least 95% identical to SEQ ID NO:38.

43. A chimeric polypeptide comprising SEQ ID NO:38.

44. A chimeric polypeptide consisting of SEQ ID NO:38.

45. A nucleic acid molecule comprising a nucleotide sequence encoding the chimeric polypeptide of any one of claims 1-44.

46. The nucleic acid molecule of claim 45, wherein the nucleic acid molecule comprises a nucleic acid sequence at least 95% identical to SEQ ID NO:2.

47. The nucleic acid molecule of claim 46, wherein the nucleic acid molecule comprises SEQ ID NO:2.

48. The nucleic acid molecule of claim 45, wherein the nucleic acid molecule comprises a nucleic acid sequence at least 95% identical to SEQ ID NO:39.

49. The nucleic acid molecule of claim 48, wherein the nucleic acid molecule comprises SEQ ID NO:39.

50. A vector comprising the nucleic acid molecule of any one of claims 45-49.

51. A method of generating an engineered cell comprising introducing into a cell the chimeric polypeptide of any one of claims 1-44, the nucleic acid molecule of any one of claims 45-49, or the vector of claim 50.

52. An engineered cell comprising the chimeric polypeptide of any one of claims 1-44, the nucleic acid molecule of any one of claims 45-49, or the vector of claim 50.

53. The engineered cell of claim 52, wherein the engineered cell is a T cell.

54. The engineered cell of claim 53, wherein the T cell is a CD4⁺ T cell, CD8⁺ T cell, iNKT cell, NKT cell, γδ T cell, or regulatory T cell.

55. The engineered cell of claim 52, wherein the engineered cell is a natural killer cell.

56. The engineered cell of claim 52, wherein the engineered cell is an induced pluripotent stem cell (iPSC).

57. The engineered cell of claim 52, wherein the engineered cell is an iPSC-derived cell.

58. The engineered cell of any one of claims 52-57, further comprising a chimeric antigen receptor (CAR).

59. The engineered cell of claim 58, wherein the chimeric polypeptide is operatively linked to the CAR.

60. The engineered cell of any one of claims 52-59, further comprising a T cell receptor (TCR).

61. The engineered cell of claim 60, wherein the chimeric polypeptide is operatively linked to the TCR.

62. A population of engineered cells comprising the engineered cell of any one of claims 52-61.

63. A method for detecting, isolating, depleting, or purifying the engineered cell of any one of claims 52-61, the method comprising contacting the engineered cell with a BCMA-binding protein.

64. The method of claim 63, wherein the BCMA-binding protein is an anti-BCMA antibody or antigen-binding fragment thereof.

65. The method of claim 63 or 64, further comprising separating the cell from a population of cells using the BCMA-binding protein.

66. The method of any one of claims 63-65, further comprising detecting the cell with an imaging agent, wherein the BCMA-binding protein is linked to the imaging agent.

67. The method of claim 63 or 64, wherein the BCMA-binding protein is linked to a cytotoxic agent.

68. The method of claim 67, wherein the BCMA-binding protein linked to a cytotoxic agent is belantamab mafodotin.

69. The method of any one of claims 63-68, wherein contacting the engineered cell with the BCMA-binding protein is performed in vitro.

70. The method of any one of claims 63-68, wherein contacting the engineered cell with the BCMA-binding protein is performed ex vivo.

71. The method of any one of claims 63-68, wherein contacting the engineered cell with the BCMA-binding protein is performed in vivo.

72. A chimeric polypeptide comprising:
(a) a tissue-type plasminogen activator (tPA) signal peptide;
(b) an extracellular domain from BCMA;
(c) a hinge region;
(d) a transmembrane domain; and
(e) an intracellular region.

73. The chimeric polypeptide of claim 72, wherein the tPA signal peptide comprises a sequence at least 95% sequence identical to SEQ ID NO:34.

74. The chimeric polypeptide of claim 73, wherein the tPA signal peptide comprises SEQ ID NO:34.

75. The chimeric polypeptide of claim 74, wherein the tPA signal peptide consists of SEQ ID NO:34.

76. The chimeric polypeptide of any one of claims 72-75, wherein the extracellular domain comprises an amino acid sequence at least 95% identical to SEQ ID NO:19.

77. The chimeric polypeptide of claim 76, wherein the extracellular domain comprises SEQ ID NO:19.

78. The chimeric polypeptide of claim 77, wherein the extracellular domain consists of SEQ ID NO:19.

79. The chimeric polypeptide of any one of claims 72-78, wherein the hinge region comprises a CD8a hinge, a PDL1 hinge, an IgG4 hinge, an IgG1 hinge, or a CD34 hinge.

80. The chimeric polypeptide of claim 79, wherein the hinge region is a hinge region from PDL1.

81. The chimeric polypeptide of claim 80, wherein the hinge region comprises SEQ ID NO:23.

82. The chimeric polypeptide of claim 81, wherein the hinge region consists of SEQ ID NO:23.

83. The chimeric polypeptide of claim 79, wherein the hinge region is a hinge region from CD8α.

84. The chimeric polypeptide of claim 83, wherein the hinge region comprises SEQ ID NO:24.

85. The chimeric polypeptide of claim 84, wherein the hinge region consists of SEQ ID NO:24.

86. The chimeric polypeptide of any one of claims 72-85, wherein the transmembrane domain is an alpha or beta chain of the T cell receptor or a transmembrane domain from CD28, CD3e, CD45, CD4, CD5, CD8, CD9, CD16, CD22, CD33, CD37, CD64, CD80, CD86, CD123, CD134, CD137 or CD154.

87. The chimeric polypeptide of any one of claims 72-86, wherein the transmembrane domain is a transmembrane domain from CD8α.

88. The chimeric polypeptide of any one of claims 72-87, wherein the transmembrane domain comprises an amino acid sequence at least 95% identical to SEQ ID NO:26.

89. The chimeric polypeptide of claim 88, wherein the transmembrane domain comprises SEQ ID NO:26.

90. The chimeric polypeptide of claim 89, wherein the transmembrane domain consists of SEQ ID NO:26.

91. The chimeric polypeptide of any one of claims 72-90, wherein the intracellular region is a portion of an intracellular region from CD8α.

92. The chimeric polypeptide of any one of claims 72-91, wherein the intracellular region comprises at most 10 amino acids.

93. The chimeric polypeptide of any one of claims 72-92, wherein the intracellular region comprises at most 6 amino acids.

94. The chimeric polypeptide of any one of claims 72-93, wherein the intracellular region comprises SEQ ID NO:30.

95. The chimeric polypeptide of claim 94, wherein the intracellular region consists of SEQ ID NO:30.

96. The chimeric polypeptide of any one of claims 72-87, wherein the chimeric polypeptide is less than or equal to 150 amino acids in length.

97. The chimeric polypeptide of any one of claims 72-96, wherein the chimeric polypeptide does not comprise a signaling domain.

98. The chimeric polypeptide of any one of claims 72-97, wherein the chimeric polypeptide does not comprise an intracellular region from BCMA.

99. The chimeric polypeptide of any one of claims 72-98, wherein the chimeric polypeptide comprises an amino acid sequence at least 95% identical to SEQ ID NO:3.

100. The chimeric polypeptide of claim 99, wherein the chimeric polypeptide comprises SEQ ID NO:3.

101. A chimeric polypeptide comprising an amino acid sequence at least 95% identical to SEQ ID NO:3.

102. A chimeric polypeptide comprising SEQ ID NO:3.

103. A chimeric polypeptide consisting of SEQ ID NO:3.

104. A nucleic acid molecule comprising a nucleotide sequence encoding the chimeric polypeptide of any one of claims 72-103.

105. The nucleic acid molecule of claim 104, wherein the nucleic acid molecule comprises a nucleic acid sequence at least 95% identical to SEQ ID NO:4.

106. The nucleic acid molecule of claim 105, wherein the nucleic acid molecule comprises SEQ ID NO:4.

107. A vector comprising the nucleic acid molecule of any one of claims 104-106.

108. A method of generating an engineered cell comprising introducing into a cell the chimeric polypeptide of any one of claims 72-103, the nucleic acid molecule of any one of claims 104-106, or the vector of claim 107.

109. An engineered cell comprising the chimeric polypeptide of any one of claims 72-103, the nucleic acid molecule of any one of claims 104-106, or the vector of claim 107.

110. The engineered cell of claim 109, wherein the engineered cell is a T cell.

111. The engineered cell of claim 110, wherein the T cell is a CD4⁺ T cell, CD8⁺ T cell, iNKT cell, NKT cell, γδ T cell, or regulatory T cell.

112. The engineered cell of claim 109, wherein the engineered cell is a natural killer cell.

113. The engineered cell of claim 109, wherein the engineered cell is an iPSC.

114. The engineered cell of claim 109, wherein the cell is an iPSC-derived cell.

115. The engineered cell of any one of claims 109-114, further comprising a CAR.

116. The engineered cell of claim 115, wherein the chimeric polypeptide is operatively linked to the CAR.

117. The engineered cell of any one of claims 109-116, further comprising a TCR.

118. The engineered cell of claim 117, wherein the chimeric polypeptide is operatively linked to the TCR.

119. A method for detecting, isolating, depleting, or purifying the engineered cell of any one of claims 109-118, the method comprising contacting the engineered cell with a BCMA-binding protein.

120. The method of claim 119, wherein the BCMA-binding protein is an anti-BCMA antibody or antigen-binding fragment thereof.

121. The method of claim 119 or 120, further comprising separating the cell from a population of cells using the BCMA-binding protein.

122. The method of claim 119 or 120, further comprising detecting the cell with an imaging agent, wherein the BCMA-binding protein is linked to the imaging agent.

123. The method of claim 119, wherein the BCMA-binding protein is linked to a cytotoxic agent.

124. The method of claim 123, wherein the BCMA-binding protein linked to a cytotoxic agent is belantamab mafodotin.

125. The method of any one of claims 119-124, wherein contacting the engineered cell with the BCMA-binding protein is performed in vitro.

126. The method of any one of claims 119-124, wherein contacting the engineered cell with the BCMA-binding protein is performed ex vivo.

127. The method of any one of claims 119-124, wherein contacting the engineered cell with the BCMA-binding protein is performed in vivo.

128. A chimeric polypeptide comprising:
(a) an extracellular domain from CD30;
(b) a transmembrane domain from CD30; and
(c) an intracellular region from BCMA.

129. The chimeric polypeptide of claim 128, wherein the extracellular domain comprises an amino acid sequence at least 95% identical to SEQ ID NO:20.

130. The chimeric polypeptide of claim 129, wherein the extracellular domain comprises SEQ ID NO:20.

131. The chimeric polypeptide of claim 130, wherein the extracellular domain consists of SEQ ID NO:20.

132. The chimeric polypeptide of any one of claims 128-131, wherein the transmembrane domain comprises an amino acid sequence at least 95% identical to SEQ ID NO:27.

133. The chimeric polypeptide of claim 132, wherein the transmembrane domain comprises SEQ ID NO:27.

134. The chimeric polypeptide of claim 132 or 133, wherein the transmembrane domain consists of SEQ ID NO:27.

135. The chimeric polypeptide of any one of claims 128-134, wherein the intracellular region comprises an amino acid sequence at least 95% identical to SEQ ID NO:33.

136. The chimeric polypeptide of claim 135, wherein the intracellular region comprises SEQ ID NO:33.

137. The chimeric polypeptide of claim 135 or 136, wherein the intracellular region consists of SEQ ID NO:33.

138. The chimeric polypeptide of any one of claims 128-137, wherein the chimeric polypeptide does not comprise a signaling domain.

139. The chimeric polypeptide of any one of claims 128-138, wherein the chimeric polypeptide does not comprise an intracellular region from CD30.

140. The chimeric polypeptide of any one of claims 128-139, wherein the chimeric polypeptide comprises an amino acid sequence at least 95% identical to SEQ ID NO:9.

141. The chimeric polypeptide of any one of claims 128-140, wherein the chimeric polypeptide comprises an amino acid sequence at least 99% identical to SEQ ID NO:9.

142. The chimeric polypeptide of any one of claims 128-141, wherein the chimeric polypeptide comprises SEQ ID NO:9.

143. A chimeric polypeptide comprising an amino acid sequence at least 95% identical to SEQ ID NO:9.

144. A chimeric polypeptide comprising SEQ ID NO:9.

145. A chimeric polypeptide consisting of SEQ ID NO:9.

146. A nucleic acid molecule comprising a nucleotide sequence encoding the chimeric polypeptide of any one of claims 128-145.

147. The nucleic acid molecule of claim 146, wherein the nucleic acid molecule comprises a nucleic acid sequence at least 95% identical to SEQ ID NO:10.

148. The nucleic acid molecule of claim 146 or 147, wherein the nucleic acid molecule comprises SEQ ID NO:10.

149. A vector comprising the nucleic acid molecule of any one of claims 146-148.

150. A method of generating an engineered cell comprising introducing into a cell the chimeric polypeptide of any one of claims 128-145, the nucleic acid molecule of any one of claims 146-148, or the vector of claim 149.

151. An engineered cell comprising the chimeric polypeptide of any one of claims 128-145, the nucleic acid molecule of any one of claims 146-148, or the vector of claim 149.

152. The engineered cell of claim 151, wherein the engineered cell is a T cell.

153. The engineered cell of claim 152, wherein the T cell is a CD4+ T cell, CD8+ T cell, iNKT cell, NKT cell, γδ T cell, or regulatory T cell.

154. The engineered cell of claim 151, wherein the engineered cell is a natural killer cell.

155. The engineered cell of claim 151, wherein the engineered cell is an iPSC.

156. The engineered cell of claim 151, wherein the engineered cell is an iPSC-derived cell.

157. The engineered cell of any one of claims 151-156, further comprising a CAR.

158. The engineered cell of claim 157, wherein the chimeric polypeptide is operatively linked to the CAR.

159. The engineered cell of any one of claims 151-156, further comprising a TCR.

160. The engineered cell of claim 159, wherein the chimeric polypeptide is operatively linked to the TCR.

161. A population of engineered cells comprising the engineered cell of any one of claims 151-160.

162. A method for detecting, isolating, depleting, or purifying the engineered cell of any one of claims 151-160, the method comprising contacting the engineered cell with a CD30-binding protein.

163. The method of claim 162, wherein the CD30-binding protein is an anti-CD30 antibody or antigen-binding fragment thereof.

164. The method of claim 162 or 163, further comprising separating the cell from a population of cells using the CD30-binding protein.

165. The method of claim 162 or 163, further comprising detecting the cell with an imaging agent, wherein the CD30-binding protein is linked to the imaging agent.

166. The method of claim 162 or 163, wherein the CD30-binding protein is linked to a cytotoxic agent.

167. The method of any one of claims 162-166, wherein contacting the engineered cell with the CD30-binding protein is performed in vitro.

168. The method of any one of claims 162-166, wherein contacting the engineered cell with the CD30-binding protein is performed ex vivo.

169. The method of any one of claims 162-166, wherein contacting the engineered cell with the CD30-binding protein is performed in vivo.

170. A chimeric polypeptide comprising:
(a) a signal peptide that is not a Her2 signal peptide;
(b) an extracellular domain from Her2; and
(c) a transmembrane domain from Her2.

171. The chimeric polypeptide of claim 170, wherein the chimeric polypeptide does not comprise an intracellular region.

172. The chimeric polypeptide of claim 170 or 171, wherein the signal peptide is a signal peptide from CD8α.

173. The chimeric polypeptide of claim 172, wherein the signal peptide comprises an amino acid sequence at least 95% identical to SEQ ID NO:35.

174. The chimeric polypeptide of claim 173, wherein the signal peptide comprises SEQ ID NO:35.

175. The chimeric polypeptide of claim 174, wherein the signal peptide consists of SEQ ID NO:35.

176. The chimeric polypeptide of any one of claims 170-175, wherein the extracellular domain comprises an amino acid sequence at least 95% identical to SEQ ID NO:21.

177. The chimeric polypeptide of claim 176, wherein the extracellular domain comprises SEQ ID NO:21.

178. The chimeric polypeptide of claim 177, wherein the extracellular domain consists of SEQ ID NO:21.

179. The chimeric polypeptide of any one of claims 170-178, wherein the transmembrane domain comprises an amino acid sequence at least 95% identical to SEQ ID NO:28.

180. The chimeric polypeptide of claim 179, wherein the transmembrane domain comprises SEQ ID NO:28.

181. The chimeric polypeptide of claim 180, wherein the transmembrane domain consists of SEQ ID NO:28.

182. The chimeric polypeptide of any one of claims 170-181, wherein the chimeric polypeptide comprises an amino acid sequence at least 95% identical to SEQ ID NO:11.

183. The chimeric polypeptide of any one of claims 170-182, wherein the chimeric polypeptide comprises an amino acid sequence at least 99% identical to SEQ ID NO:11.

184. The chimeric polypeptide of any one of claims 170-183, wherein the chimeric polypeptide comprises SEQ ID NO:11.

185. A chimeric polypeptide comprising an amino acid sequence at least 95% identical to SEQ ID NO:11.

186. A chimeric polypeptide comprising SEQ ID NO:11.

187. A chimeric polypeptide consisting of SEQ ID NO:11.

188. A nucleic acid molecule comprising a nucleotide sequence encoding the chimeric polypeptide of any one of claims 170-187.

189. The nucleic acid molecule of claim 188, wherein the nucleic acid molecule comprises a nucleic acid sequence at least 95% identical to SEQ ID NO:12.

190. The nucleic acid molecule of claim 189, wherein the nucleic acid molecule comprises SEQ ID NO:12.

191. A vector comprising the nucleic acid molecule of any one of claims 188-190.

192. A method of generating an engineered cell comprising introducing into a cell the chimeric polypeptide of any one of claims 170-187, the nucleic acid molecule of any one of claims 188-190, or the vector of claim 191.

193. An engineered cell comprising the chimeric polypeptide of any one of claims 170-187, the nucleic acid molecule of any one of claims 188-190, or the vector of claim 191.

194. The engineered cell of claim 193, wherein the engineered cell is a T cell.

195. The engineered cell of claim 194, wherein the T cell is a CD4+ T cell, CD8+ T cell, iNKT cell, NKT cell, γδ T cell, or regulatory T cell.

196. The engineered cell of claim 193, wherein the engineered cell is a natural killer cell.

197. The engineered cell of claim 193, wherein the engineered cell is an iPSC.

198. The engineered cell of claim 193, wherein the engineered cell is an iPSC-derived cell.

199. The engineered cell of any one of claims 193-198, further comprising a CAR.

200. The engineered cell of claim 199, wherein the chimeric polypeptide is operatively linked to the CAR.

201. The engineered cell of any one of claims 193-198, further comprising a TCR.

202. The engineered cell of claim 201, wherein the chimeric polypeptide is operatively linked to the TCR.

203. A population of engineered cells comprising the engineered cell of any one of claims 193-202.

204. A method for detecting, isolating, depleting, or purifying the engineered cell of any one of claims 193-202, the method comprising contacting the engineered cell with a Her2-binding protein.

205. The method of claim 204, wherein the Her2-binding protein is an anti-Her2 antibody or antigen-binding fragment thereof.

206. The method of claim 204 or 205, further comprising separating the cell from a population of cells using the Her2-binding protein.

207. The method of claim 204 or 205, further comprising detecting the cell with an imaging agent, wherein the Her2-binding protein is linked to the imaging agent.

208. The method of claim 204 or 205, wherein the Her2-binding protein is linked to a cytotoxic agent.

209. The method of claim 208, wherein the Her2-binding protein linked to the cytotoxic agent is trastuzumab emtansine.

210. The method of any one of claims 204-209, wherein contacting the engineered cell with the Her2-binding protein is performed in vitro.

211. The method of any one of claims 204-209, wherein contacting the engineered cell with the Her2-binding protein is performed ex vivo.

212. The method of any one of claims 204-209, wherein contacting the engineered cell with the Her2-binding protein is performed in vivo.

213. An engineered immune cell comprising a nucleic acid encoding a Trop2 polypeptide.

214. The engineered immune cell of claim 213, wherein the Trop2 polypeptide comprises SEQ ID NO:15.

215. The engineered immune cell of claim 213 or 214, wherein the nucleic acid comprises a nucleic acid sequence at least 95% identical to SEQ ID NO:16.

216. The engineered immune cell of claim 215, wherein the nucleic acid comprises SEQ ID NO:16.

217. The engineered immune cell of any one of claims 213-216, wherein the engineered immune cell is a T cell.

218. The engineered immune cell of claim 217, wherein the T cell is a CD4+ T cell, CD8+ T cell, iNKT cell, NKT cell, γδ T cell, or regulatory T cell.

219. The engineered immune cell of any one of claims 213-216, wherein the engineered immune cell is a natural killer cell.

220. The engineered immune cell of any one of claims 213-219, further comprising a CAR.

221. The engineered immune cell of claim 220, wherein the chimeric polypeptide is operatively linked to the CAR.

222. The engineered immune cell of any one of claims 213-216, further comprising a TCR.

223. The engineered immune cell of claim 222, wherein the chimeric polypeptide is operatively linked to the TCR.

224. A population of engineered cells comprising the engineered immune cell of any one of claims 213-223.

225. A method for detecting, isolating, depleting, or purifying the engineered cell of any one of claims 213-223, the method comprising contacting the engineered cell with a Trop2-binding protein.

226. The method of claim 225, wherein the Trop2-binding protein is an anti-Trop2 antibody or antigen-binding fragment thereof.

227. The method of claim 225 or 226, further comprising separating the cell from a population of cells using the Trop2-binding protein.

228. The method of claim 225 or 226, further comprising detecting the cell with an imaging agent, wherein the Trop2-binding protein is linked to the imaging agent.

229. The method of claim 225 or 226, wherein the Trop2-binding protein is linked to a cytotoxic agent.

230. The method of claim 229, wherein the Trop2-binding protein linked to the cytotoxic agent is sacituzumab govitecan.

231. The method of any one of claims 225-230, wherein contacting the engineered cell with the Trop2-binding protein is performed in vitro.

232. The method of any one of claims 225-230, wherein contacting the engineered cell with the Trop2-binding protein is performed ex vivo.

233. The method of any one of claims 225-230, wherein contacting the engineered cell with the Trop2-binding protein is performed in vivo.

234. A chimeric polypeptide comprising:
(a) a signal peptide;
(b) an extracellular region comprising (i) EGFR domain III and (ii) a portion of EGFR domain IV having a length of less than 100 amino acids;
(c) a hinge region; and
(d) a transmembrane domain.

235. The chimeric polypeptide of claim 234, wherein the portion of EGFR domain IV has a length of less than 50 amino acids.

236. The chimeric polypeptide of claim 235, wherein the portion of EGFR domain IV has a length of 33 amino acids.

237. The chimeric polypeptide of any one of claims 234-236, wherein the signal peptide is a signal peptide from GM-CSFRα.

238. The chimeric polypeptide of claim 237, wherein the signal peptide comprises an amino acid sequence at least 95% identical to SEQ ID NO:36.

239. The chimeric polypeptide of claim 238, wherein the signal peptide comprises SEQ ID NO:36.

240. The chimeric polypeptide of claim 239, wherein the signal peptide consists of SEQ ID NO:36.

241. The chimeric polypeptide of any one of claims 234-240, wherein the extracellular region comprises an amino acid sequence at least 95% identical to SEQ ID NO:22.

242. The chimeric polypeptide of claim 241, wherein the extracellular region comprises SEQ ID NO:22.

243. The chimeric polypeptide of claim 242, wherein the extracellular region consists of SEQ ID NO:22.

244. The chimeric polypeptide of any one of claims 234-243, wherein the hinge region is a hinge region from CD8.

245. The chimeric polypeptide of claim 244, wherein the hinge region comprises an amino acid sequence at least 95% identical to SEQ ID NO:37.

246. The chimeric polypeptide of claim 245, wherein the hinge region comprises SEQ ID NO:37.

247. The chimeric polypeptide of claim 246, wherein the hinge region consists of SEQ ID NO:37.

248. The chimeric polypeptide of any one of claims 234-247, wherein the transmembrane domain comprises an amino acid sequence at least 95% identical to SEQ ID NO:26.

249. The chimeric polypeptide of claim 248, wherein the transmembrane domain comprises SEQ ID NO:26.

250. The chimeric polypeptide of claim 249, wherein the transmembrane domain consists of SEQ ID NO:26.

251. The chimeric polypeptide of any one of claims 234-250, wherein the chimeric polypeptide does not comprise a signaling domain.

252. The chimeric polypeptide of any one of claims 234-251, wherein the chimeric polypeptide does not comprise an intracellular region from EGFR.

253. The chimeric polypeptide of any one of claims 234-252, wherein the chimeric polypeptide comprises an amino acid sequence at least 95% identical to SEQ ID NO:15.

254. The chimeric polypeptide of any one of claims 234-253, wherein the chimeric polypeptide comprises an amino acid sequence at least 99% identical to SEQ ID NO:15.

255. The chimeric polypeptide of any one of claims 234-254, wherein the chimeric polypeptide comprises SEQ ID NO:15.

256. A chimeric polypeptide comprising an amino acid sequence at least 95% identical to SEQ ID NO:15.

257. A chimeric polypeptide comprising SEQ ID NO:15.

258. A chimeric polypeptide consisting of SEQ ID NO:15.

259. A nucleic acid molecule comprising a nucleotide sequence encoding the chimeric polypeptide of any one of claims 234-258.

260. The nucleic acid molecule of claim 259, wherein the nucleic acid molecule comprises a nucleic acid sequence at least 95% identical to SEQ ID NO:16.

261. The nucleic acid molecule of claim 260, wherein the nucleic acid molecule comprises SEQ ID NO:16.

262. A vector comprising the nucleic acid molecule of any one of claims 259-261.

263. A method of generating an engineered cell comprising introducing into a cell the chimeric polypeptide of any one of claims 234-258, the nucleic acid molecule of any one of claims 259-261, or the vector of claim 262.

264. An engineered cell comprising the chimeric polypeptide of any one of claims 234-258, the nucleic acid molecule of any one of claims 259-261, or the vector of claim 262.

265. The engineered cell of claim 264, wherein the engineered cell is a T cell.

266. The engineered cell of claim 265, wherein the T cell is a CD4+ T cell, CD8+ T cell, iNKT cell, NKT cell, γδ T cell, or regulatory T cell.

267. The engineered cell of claim 264, wherein the engineered cell is a natural killer cell.

268. The engineered cell of claim 264, wherein the engineered cell is an iPSC.

269. The engineered cell of claim 264, wherein the engineered cell is an iPSC-derived cell.

270. The engineered cell of any one of claims 264-269, further comprising a CAR.

271. The engineered cell of claim 270, wherein the chimeric polypeptide is operatively linked to the CAR.

272. The engineered cell of any one of claims 264-271, further comprising a T cell receptor (TCR).

273. The engineered cell of claim 264, wherein the chimeric polypeptide is operatively linked to the TCR.

274. A population of engineered cells comprising the engineered cell of any one of claims 264-273.

275. A method for detecting, isolating, depleting, or purifying the engineered cell of any one of claims 264-273, the method comprising contacting the engineered cell with an EGFR-binding protein.

276. The method of claim 275, wherein the EGFR-binding protein is an anti-EGFR antibody or antigen-binding fragment thereof.

277. The method of claim 275 or 276, further comprising separating the cell from a population of cells using the EGFR-binding protein.

278. The method of claim 275 or 276, further comprising detecting the cell with an imaging agent, wherein the EGFR-binding protein is linked to the imaging agent.

279. The method of claim 275 or 276, wherein the EGFR-binding protein is linked to a cytotoxic agent.

280. The method of any one of claims 275-279, wherein contacting the engineered cell with the EGFR-binding protein is performed in vitro.

281. The method of any one of claims 275-279, wherein contacting the engineered cell with the EGFR-binding protein is performed ex vivo.

282. The method of any one of claims 275-279, wherein contacting the engineered cell with the EGFR-binding protein is performed in vivo.

283. A method for detecting, isolating, depleting, or purifying a cell, the cell comprising:
(a) an extracellular domain from B-cell maturation antigen (BCMA);
(b) a hinge region from programmed cell death 1 ligand 1 (PDL1);
(c) a transmembrane domain; and
(d) an intracellular region,
the method comprising contacting the cell with a BCMA-binding protein.

284. A method for detecting, isolating, depleting, or purifying a cell, the cell comprising:
(a) a tissue-type plasminogen activator (tPA) signal peptide;
(b) an extracellular domain from BCMA;
(c) a hinge region;
(d) a transmembrane domain; and
(e) an intracellular region,
the method comprising contacting the cell with a BCMA-binding protein.

285. A method for detecting, isolating, depleting, or purifying a cell, the cell comprising:
(a) an extracellular domain from CD30;
(b) a transmembrane domain from CD30;
(c) an intracellular region from BCMA,
the method comprising contacting the cell with a CD30-binding protein.

286. A method for detecting, isolating, depleting, or purifying a cell, the cell comprising:
(a) a signal peptide that is not a Her2 signal peptide;
(b) an extracellular domain from Her2;
(c) a transmembrane domain from Her2,
the method comprising contacting the cell with a Her2-binding protein.

287. A method for detecting, isolating, depleting, or purifying an engineered immune cell, the engineered immune cell comprising a Trop2 polypeptide, the method comprising contacting the cell with a Trop2-binding protein.

288. A method for detecting, isolating, depleting, or purifying a cell, the cell comprising:
(a) a signal peptide;
(b) an extracellular region comprising (i) EGFR domain III and (ii) a portion of EGFR domain IV having a length of less than 100 amino acids;
(c) a hinge region; and
(d) a transmembrane domain,
the method comprising contacting the cell with an EGFR-binding protein.

289. An expression construct, comprising sequence that encodes a CD30 fusion protein, wherein the fusion protein comprises at least part of the CD30 extracellular domain fused to an intracellular domain that comprises endocytosis or internalization activity.

290. The expression construct of claim 289, wherein the intracellular domain is from B-cell maturation antigen (BCMA), trop-2, CD317, CD3gamma, CD4, CD79b, low density lipoprotein receptor, CD19, CD22, CD25, CD33, or a combination thereof.

291. The expression construct of claim 289 or 290, wherein the CD30 fusion protein comprises the CD30 transmembrane domain.

292. The expression construct of any one of claims 289-291, wherein the fusion protein comprises SEQ ID NO:47.

293. The expression construct of any one of claims 289-292, wherein the sequence that encodes the fusion protein comprises SEQ ID NO:48.

294. An isolated cell, comprising the expression construct of any one of claims 289-293.

295. The cell of claim 294, wherein the cell is an immune cell.

296. The cell of claim 294 or 295, wherein the cell is an αβ T cell, γδ T cell, NK cell, NKT cell, monocyte, macrophages, B cell, mesenchymal stem cell (MSC) cell, hematopoietic stem cell (HSC), hematopoietic cell, iPSC, or a mixture thereof.

297. The cell of any one of claims 294-296, wherein the cell expresses one or more heterologous proteins other than the CD30 fusion protein.

298. The cell of claim 297, wherein the heterologous protein is a therapeutic protein, cytokine, fusion of a cytokine and cytokine receptor, safety switch, or a mixture thereof.

299. The cell of claim 298, wherein the therapeutic protein is an engineered antigen receptor or antibody.

300. The cell of claim 299, wherein the engineered antigen receptor targets a cancer antigen.

301. The cell of claim 299 or 300, wherein the engineered antigen receptor is a chimeric antigen receptor, T cell receptor, or B-cell receptor.

302. The cell of any one of claims 8-21, wherein the cell expresses heterologous BCL6 and BCL2L1 genes.

303. An isolated population of any one of the cells of claims 294-302, said population comprised in a suitable medium.

304. The population of claim 303, housed in a depository.

305. The population of claim 303 or 304, wherein the population is cryopreserved.

306. A method of identifying CD30-positive cells that are transduced or transfected with (1) a heterologous CD30 gene; (2) a CD30 fusion protein comprising at least part of the CD30 extracellular domain; or (3) a combination of heterologous BCL6 and one or more Bcl2 family genes, comprising the steps of:
transducing or transfecting cells with (1) a heterologous CD30 gene; (2) a CD30 fusion protein comprising at least part of the CD30 extracellular domain; or (3) a combination of heterologous BCL6 and one or more Bcl2 family genes;
exposing the cells to an effective amount of an agent that binds CD30; and
directly or indirectly detecting the binding of the agent to CD30 on the surface of the cells.

307. The method of claim 306, wherein the exposing and detecting steps occur during and/or after manufacture of the cells.

308. The method of claim 306 or 307, further comprising the step of manufacturing the cells.

309. The method of any one of claims 306-308, further defined as:
transfecting or transducing immune cells with the CD30 fusion protein;
exposing the immune cells to an effective amount of an antibody or antibody-drug conjugate that binds CD30; and
directly or indirectly detecting the binding of the antibody or antibody-drug conjugate to CD30 on the surface of the cells.

310. The method of any one of claims 306-309, further defined as:
transfecting or transducing immune cells with the heterologous BCL6 and one or more Bcl2 family genes;
exposing the immune cells to an effective amount of an antibody or antibody-drug conjugate that binds CD30; and
directly or indirectly detecting the binding of the antibody or antibody-drug conjugate to CD30 on the surface of the cells.

311. The method of any one of claims 306-310, wherein the method occurs in vitro.

312. The method of any one of claims 306-310, wherein at least part of the method occurs in vivo.

313. The method of any one of claims 306-312, wherein the cells express one or more heterologous proteins other than the CD30 fusion protein.

314. The method of claim 313, wherein the heterologous protein is a therapeutic protein, cytokine, fusion of a cytokine and cytokine receptor, safety switch, or a mixture thereof.

315. The method of claim 314, wherein the therapeutic protein is an engineered antigen receptor.

316. The method of any one of claims 306-315, further comprising the step of transfecting or transforming the immune cells with a heterologous protein other than the heterologous CD30 or the CD30 fusion protein; or the BCL6 and one or more Bcl2 family genes.

317. The method of claim 316, wherein the CD30 fusion protein and the heterologous protein other than the CD30 fusion protein or the BCL6 and one or more Bcl2 family genes are expressed from the same vector.

318. The method of claim 316, wherein the CD30 fusion protein and the heterologous protein other than the CD30 fusion protein or the BCL6 and one or more Bcl2 family genes are expressed from different vectors.

319. A method of manufacturing immune cells for adoptive cell therapy, comprising the steps of:
(a) transducing or transfecting immune cells with (1) heterologous CD30 protein; (2) a CD30 fusion protein; or (3) a combination of heterologous BCL6 and one or more Bcl2 family genes, wherein the immune cells respectively express (1) CD30; (2) the CD30 fusion protein; or (3) CD30; and
(b) transducing or transfecting the immune cells with one or more therapeutic proteins.

320. The method of claim 319, wherein the step (a) occurs prior to, at the same time as, or subsequent to step (b).

321. The method of claim 319, wherein the transforming or transfecting in step (a) is the same transforming or transfecting in step (b).

322. The method of any one of claims 319-321, wherein (1), (2), or (3) are on the same vector as the therapeutic protein.

323. The method of any one of claims 319-321, wherein (1), (2), or (3) are on a different vector as the therapeutic protein.

324. The method of any one of claims 50-54, wherein following step (a), immune cells from the method are analyzed for the presence of CD30 expressed on the surface of the immune cells.

325. The method of claim 324, wherein the analyzing comprises flow cytometry, polymerase chain reaction, or a combination thereof.

326. The method of any one of claims 319-325, further comprising the step of administering immune cells produced from the method to an individual in need thereof.

327. The method of claim 326, wherein the immune cells are monitored in the individual.

328. The method of claim 326, wherein the immune cells are monitored in the individual using an agent that binds CD30.

329. The method of claim 328, wherein the agent is an antibody or antibody-drug conjugate.

330. The method of any one of claims 319-329, wherein the individual exhibits one or more deleterious effects from the immune cells, and the individual is administered an effective amount of an agent that binds CD30.

331. The method of claim 330, wherein the individual exhibits toxicity from the immune cells, and the individual is administered an effective amount of an agent that binds CD30.

332. The method of claim 330, wherein the individual exhibits graft-versus-host disease (GVHD) from the immune cells, and the individual is administered an effective amount of an agent that binds CD30.

333. The method of any one of claims 329-332, wherein the individual is no longer in need of the immune cells, and the individual is administered an effective amount of an agent that binds CD30.

334. A method of reducing or preventing one or more deleterious effects from a cellular therapy in an individual, comprising the step of targeting CD30 expressed on the surface of cells of the cellular therapy.

335. The method of claim 334, further defined as administering to the individual an effective amount of one or more agents that bind CD30 expressed on the cells.

336. The method of claim 335, wherein the agent is an antibody or antibody-drug conjugate.

337. The method of claim 336, wherein the antibody is a monoclonal antibody.

338. The method of any one of claims 334-337, wherein the deleterious effects comprise GVHD, cytokine release syndrome, or immune effector cell-associated neurotoxicity syndrome.

339. The method of any one of claims 334-338, wherein the CD30 is the entire CD30 protein.

340. The method of any one of claims 334-338, wherein the CD30 is a fragment of the entire CD30 protein that comprises at least part of the extracellular domain.

341. The method of any one of claims 334-339, wherein the CD30 is expressed naturally on the immune cells or is heterologously expressed naturally on the immune cells.

342. The method of any one of claims 334-339, wherein the CD30 is expressed on the cells as a result of the cells expressing heterologous BCL6 and one or more Bcl2 family genes.

343. The method of any one of claims 334-340, wherein the CD30 is a CD30 fusion protein.

344. The method of claim 343, wherein the fusion protein comprises at least part of the CD30 extracellular domain fused to an intracellular domain that comprises endocytosis or internalization activity.

345. The method of claim 344, wherein the intracellular domain is from B-cell maturation antigen (BCMA), trop-2, CD317, CD3gamma, CD4, CD79b, or a combination thereof.

346. The method of claim 344 or 345, wherein the CD30 fusion protein comprises the CD30 transmembrane domain.

347. The method of any one of claims 334-346, wherein prior to the targeting step, the cells of the individual were monitored in vivo.

348. The method of claim 347, wherein the cells are monitored using one or more agents that target CD30.

349. The method of claim 348, wherein the agent that targets CD30 is an antibody used in a low enough amount not to inhibit the therapeutic cells.

350. The method of any one of claims 334-349, wherein the cellular therapy is autologous or allogeneic with respect to the individual.

351. A method of inhibiting the activity of cells, comprising the step of exposing cells that are transduced or transfected with a combination of heterologous BCL6 and one or more Bcl2 family genes to an effective amount of an agent that binds CD30.

352. The method of claim 351, wherein the inhibiting of activity is further defined as inducing apoptosis of the cells.

353. The method of claim 351 or 352, wherein the method further comprises detecting binding of the agent to CD30 expressed on the cells.

354. The method of any one of claims 351-353, wherein the method further comprises transducing or transfecting cells with the heterologous BCL6 and with one or more Bcl2 family genes.

355. The method of any one of claims 351-354, wherein the cells are T cells.

356. The method of any one of claims 351-355, wherein the one or more Bcl2 family genes is BCL2L1.

357. A method of inhibiting the activity of cells, comprising the step of exposing cells that are transduced or transfected with a CD30 fusion protein comprising at least part of the CD30 extracellular domain to an effective amount of an agent that binds CD30.

358. The method of claim 357, wherein the inhibiting of activity is further defined as inducing apoptosis of the cells.


SEQUENCE LISTING (51 sequences)

SEQ ID NO: 1 - protein, 84 aa, Artificial Sequence
  Description of Artificial Sequence Synthetic polypeptide
MLQMAGQCSQNEYFDSLLHACIPCQLRCSSNTPPLTCQRYCNASVTNSVKGTNAHPPNERTHLVILGAILLCLGVALTFIFRLR

SEQ ID NO: 2 - DNA, 255 bp, Artificial Sequence
  Description of Artificial Sequence Synthetic polynucleotide
atgctccagatggctggccagtgttcgcagaacgagtacttcgacagtcttctacacgcctgtattccttgtcagctgcgttgctctagcaacacgcccccactgacctgccagcgttactgcaatgcttccgtcaccaactccgtgaagggcaccaacgcgcatccgcccaacgagcgcacacacctggtgatcttgggtgccatcctgctgtgcctgggggtggcactcacttttatcttccgcctgcggtaa

SEQ ID NO: 3 - protein, 149 aa, Artificial Sequence
  Description of Artificial Sequence Synthetic polypeptide
MDAMKRGLCCVLLLCGAVFVSPSQEIHARFRRGARMLQMAGQCSQNEYFDSLLHACIPCQLRCSSNTPPLTCQRYCNASVTNSVKGTNAPTIASQPLSLRPEACRPAAGGAVHTRGLDFACDIYIWAPLAGTCGVLLLSLVITLYCWVR

SEQ ID NO: 4 - DNA, 450 bp, Artificial Sequence
  Description of Artificial Sequence Synthetic polynucleotide
atggatgccatgaagcgcggtctgtgctgcgtgttgctgctgtgcggggccgtgttcgtctcaccctcccaggagatccacgcccgcttcagacgtggtgcccgtatgctgcagatggctggccagtgctctcagaacgagtacttcgacagccttttgcatgcttgtattccgtgccagctgaggtgttcgagcaacacgcccccactcacctgtcagcgttactgcaatgcgtccgtaaccaactccgtgaagggcactaacgcgcctaccatcgcgagtcaaccgctgtctctgcggcccgaggcctgccgacccgcggccggcggcgctgttcacactcgcggcctggactttgcatgcgacatctatatttgggcacctcttgctggaacatgcggggtgctgctcctctccctagtgatcaccctgtactgttgggtccgctaa

SEQ ID NO: 5 - protein, 94 aa, Artificial Sequence
  Description of Artificial Sequence Synthetic polypeptide
MLQMAGQCSQNEYFDSLLHACIPCQLRCSSNTPPLTCQRYCNASVTNSVKGTNAHPPNERTHLVILGAILLCLGVALTFIFRASTSYDYCRVPG

SEQ ID NO: 6 - DNA, 282 bp, Artificial Sequence
  Description of Artificial Sequence Synthetic polynucleotide
atgctccagatggctggccagtgttcgcagaacgagtacttcgacagtcttctacacgcctgtattccttgtcagctgcgttgctctagcaacacgcccccactgacctgccagcgttactgcaatgcttccgtcaccaactccgtgaagggcaccaacgcgcatccgcccaacgagcgcacacacctggtgatcttgggtgccatcctgctgtgcctgggggtggcactcacttttatcttccgcgcatccacgagttacgattactgtcgagttccaggg

SEQ ID NO: 7 - protein, 92 aa, Artificial Sequence
  Description of Artificial Sequence Synthetic polypeptide
MLQMAGQCSQNEYFDSLLHACIPCQLRCSSNTPPLTCQRYCNASVTNSVKGTNAHPPNERTHLVILGAILLCLGVALTFIFRASDKQTLLPG

SEQ ID NO: 8 - DNA, 279 bp, Artificial Sequence
  Description of Artificial Sequence Synthetic polynucleotide
atgctccagatggctggccagtgttcgcagaacgagtacttcgacagtcttctacacgcctgtattccttgtcagctgcgttgctctagcaacacgcccccactgacctgccagcgttactgcaatgcttccgtcaccaactccgtgaagggcaccaacgcgcatccgcccaacgagcgcacacacctggtgatcttgggtgccatcctgctgtgcctgggggtggcactcacttttatcttccgcgcatccgacaagcagacccttctccctgggtga

SEQ ID NO: 9 - protein, 514 aa, Artificial Sequence
  Description of Artificial Sequence Synthetic polypeptide
MRVLLAALGLLFLGALRAFPQDRPFEDTCHGNPSHYYDKAVRRCCYRCPMGLFPTQQPQRPTDCRKQCEPDYYLDEADRCTACVTCSRDDLVEKTPCAWNSSRVCECRPGMFCSTSAVNSCARCFFHSVCPAGMIVKFPGTAQKNTVCEPASPGVSPACASPENCKEPSSGTIPQAKPTPVSPATSSASTMPVRGGTRLAQEAASKLTRAPDSPSSVGRPSSDPGLSPTQPCPEGSGDCRKQCEPDYYLDEAGRCTACVSCSRDDLVEKTPCAWNSSRTCECRPGMICATSATNSCARCVPYPICAAETVTKPQDMAEKDTTFEAPPLGTQPDCNPTPENGEAPASTSPTQSLLVDSQASKTLPIPTSAPVALSSTGKPVLDAGPVLFWVILVLVVVVGSSAFLLCHRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 10 - DNA, 1545 bp, Artificial Sequence
  Description of Artificial Sequence Synthetic polynucleotide
atgcgagtcctcctggccgcgttggggctcttgttccttggggcacttcgagcctttccacaggatcgaccttttgaagatacttgtcacggaaacccttctcactactacgataaagcggtccgacgatgctgctaccgatgccctatgggacttttcccgacgcagcaatgcccacagcggcctacggactgtagaaagcaatgcgagccggactactatctggacgaagcagatcggtgtactgcctgcgtaacatgctcccgggatgacctggtggaaaagaccccctgcgcttggaactccagtagagtatgcgaatgccgaccagggatgttctgcagcacgagcgcagttaactcatgtgccaggtgttttttccattctgtatgtcctgccgggatgattgttaagttcccaggtacagctcaaaagaacacggtatgcgaacccgctagtccgggagtttccccggcctgcgccagcccagagaattgcaaggagccgtctagcggtacaatcccacaagctaagccgacgccggtcagcccggcgacttcatccgcctcaacaatgcccgtccggggtgggacaagactcgcgcaggaagcggctagcaagttgacacgagcccccgattccccttcaagtgtaggcagacctagttccgaccctggccttagcccaacgcaaccctgcccagagggatccggagactgtcggaaacaatgcgagcccgactattacttggatgaagccggtcgctgtaccgcatgtgtgtcctgcagccgcgatgacctggtcgagaaaacaccatgtgcatggaatagcagtcgcacctgcgagtgccgaccgggaatgatctgtgccacctcagcaaccaactcttgtgccaggtgcgtaccatatcccatctgcgcggctgagaccgtaacaaaacctcaagacatggccgaaaaagacaccactttcgaagcgccgcctctgggcactcaaccagactgcaatcctacgccagaaaacggggaagcacccgcgtcaacctcccccacacaatctttgctcgtagactctcaggcttccaaaacactgccaataccaacttccgctccggtggctctgagctctaccggcaaacccgtgcttgacgccgggccagtcctgttctgggttatattggtgctcgtagtcgtagtaggctccagcgcctttctgctctgtcaccggaagatcaattccgaacctttgaaagacgagtttaagaacaccgggagtggcctcctcggaatggctaatatcgacttggagaagagccgcactggggacgaaatcattttgcctcgcgggcttgaatacacagtcgaagagtgcacgtgtgaagactgcattaaatcaaaaccgaaggtggacagcgatcattgtttccccttgcccgctatggaagaaggtgcaactatcctcgtaacaaccaaaactaacgattattgtaaaagcctcccggcggctctctctgcgacggaaatagaaaaatcaatctctgcaagg

SEQ ID NO: 11 - protein, 190 aa, Artificial Sequence
  Description of Artificial Sequence Synthetic polypeptide
MALPVTALLLPLALLLHAARPLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIISAVVGILLVVVLGVVFGILIKR

SEQ ID NO: 12 - DNA, 570 bp, Artificial Sequence
  Description of Artificial Sequence Synthetic polynucleotide
atggccctgccagtgaccgccctgctgctgccactggcactgctgctgcacgcagcaaggccactcgcttgtcaccagctgtgcgctcgcggccactgttggggccccggtcctacccagtgcgtcaactgttcgcagttcctaagagggcaggagtgcgtggaggaatgccgtgtgctgcagggtctgccacgcgagtacgtgaacgcccgtcactgtctgccatgtcaccccgagtgccagccccagaacgggagcgtgacctgctttggaccggaggcggaccagtgcgtggcttgtgcccattacaaggatccaccgttctgcgtggcgaggtgtccctctggcgtgaagcccgatctgagctacatgcctatatggaaatttccggacgaggaaggggcttgtcagccttgcccgatcaactgtactcattcctgcgtggacttggatgacaagggatgcccggcggagcagcgcgcgtccccgttgaccagcattatctccgcagtggtgggcatcctgttggtcgtcgttctgggcgtcgtgttcggtattctcatcaagcgg

SEQ ID NO: 13 - protein, 323 aa, Homo sapiens
MARGPGLAPPPLRLPLLLLVLAAVTGHTAAQDNCTCPTNKMTVCSPDGPGGRCQCRALGSGMAVDCSTLTSKCLLLKARMSAPKNARTLVRPSEHALVDNDGLYDPDCDPEGRFKARQCNQTSVCWCVNSVGVRRTDKGDLSLRCDELVRTHHILIDLRHRPTAGAFNHSDLDAELRRLFRERYRLHPKFVAAVHYEQPTIQIELRQNTSQKAAGDVDIGDAAYYFERDIKGESLFQGRGGLDLRVRGEPLQVERTLIYYLDEIPPKFSMKRLTAGLIAVIVVVVVALVAGMAVLVITNRRKSGKYKKVEIKELGELRKEPSL

SEQ ID NO: 14 - DNA, 972 bp, Homo sapiens
atggcgagaggtcccgggttggcacccccaccgttgcggcttcctcttttgcttctcgtgcttgccgccgtcactgggcacacggctgctcaagataactgcacttgccccactaacaaaatgacagtttgcagccccgatggacctggtggtcgctgtcagtgtagagcgcttggatctggaatggctgtagattgctccaccctcacgagtaaatgtttgctgttgaaagcaaggatgtctgcacctaaaaacgcccgcacacttgttcgcccctctgaacacgctcttgttgataatgatggtctgtatgatcccgattgcgatcccgagggccgctttaaagcaaggcaatgcaatcaaacgtccgtttgctggtgcgtcaactcagttggtgtacggcgcactgataaaggggacctgtccctccgctgcgatgaattggttcgcacccaccacatcctcatagatctcaggcaccgaccaacagctggagcattcaaccactccgacctcgacgctgagctgcggcggttgtttagggaacggtaccggctccatccgaagtttgtggccgcagttcactatgagcagcccactatccagattgagttgcgacagaacacgagccagaaggcggctggtgacgtggatataggcgatgctgcctactacttcgaacgggatataaagggagagagtctctttcagggccgagggggcctcgatctgagggtaagaggcgaacctctccaggtcgagcgcacgctgatctactacctggatgagatcccgcccaaatttagcatgaaacgacttacagccggactcatagcggtcatagttgttgttgtcgtggccctggtcgcaggtatggcggtcctcgtcatcacgaacaggagaaagagcggtaaatataagaaagtggagattaaggagcttggggaactgcgcaaagaaccctcattgtaa

SEQ ID NO: 15 - protein, 288 aa, Artificial Sequence
  Description of Artificial Sequence Synthetic polypeptide
MLLLVTSLLLCELPHPAFLRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKSQPLSLRPEACRPAAGGAVHTRGLDFACDIYIWAPLAGTCGVLLLSLVITLYCWVRGDRYQAYR

SEQ ID NO: 16 - DNA, 867 bp, Artificial Sequence
  Description of Artificial Sequence Synthetic polynucleotide
atgctgctgctggtgaccagcctgctgctgtgcgagctgccacaccctgccttcctgaggaaagtgtgtaatggcatcggcatcggcgagtttaaggacagcctgtccatcaacgccacaaatatcaagcacttcaagaactgtacctctatcagcggcgacctgcacatcctgccagtggccttcagaggcgattcctttacacacaccccaccactggacccacaggagctggatatcctgaagacagtgaaggagatcaccggcttcctgctgatccaggcatggccagagaacaggacagatctgcacgcctttgagaatctggagatcatcagaggcaggaccaagcagcacggccagttctctctggccgtggtgagcctgaacatcacatccctgggcctgcgctctctgaaggagatcagcgacggcgatgtgatcatctccggcaacaagaatctgtgctatgccaacaccatcaattggaagaagctgtttggcacatctggccagaagaccaagatcatcagcaaccgcggcgagaattcctgcaaggcaaccggacaggtgtgccacgcactgtgtagccctgagggatgttggggaccagagccacgcgactgcgtgtcctgtaggaacgtgtctaggggaagggagtgcgtggataagtcacagcctttgtcactgcgaccggaagcatgcaggccagcggcgggtggggcagtacatacgcgaggtctggactttgcgtgtgatatttatatatgggcaccactcgccggtacgtgcggggttctcttgctctcacttgtgataacgctttattgttgggtacgataaggtgaccgatatcaagcttatcga

SEQ ID NO: 17 - protein, 184 aa, Homo sapiens
MLQMAGQCSQNEYFDSLLHACIPCQLRCSSNTPPLTCQRYCNASVTNSVKGTNAILWTCLGLSLLLSLAVFVLMFLLRKINSEPLKDEFKNTGSGLLGMANIDLEKSRTGDELLLPRGLEYTVEECTCEDCIKSKPKVDSDHCFPLPAMEEGATILVTTKTNDYCKSLPAALSATEIEKSISAR

SEQ ID NO: 18 - DNA, 555 bp, Homo sapiens
atgctgcagatggctggccagtgctctcagaacgagtatttcgacagtctattgcatgcctgcatcccgtgtcagctgaggtgttctagcaacacgcctcccctgacctgccagcgttactgtaatgcttccgtcaccaactccgtgaagggcaccaacgcgatcctttggacctgcctgggcctcagcctcatcatctctctggccgtgttcgtcctgatgtttttactccgcaagattaattccgagccactgaaggacgagttcaaaaacactggctccggtctgttgggaatggccaacattgacctggagaagtcgcgcaccggtgatgaaatcatcctgccgcgtgggttggagtacaccgtggaggagtgcacttgcgaggactgtatcaagtccaagcccaaggttgattcagaccactgtttccccctgcccgccatggaagagggcgcaaccatcctggtgacaaccaagactaacgactactgtaaaagccttcctgcggcgctgagcgccacggagattgagaaatccatctcggctcggtaa

SEQ ID NO: 19 - protein, 54 aa, Artificial Sequence
  Description of Artificial Sequence Synthetic polypeptide
MLQMAGQCSQNEYFDSLLHACIPCQLRCSSNTPPLTCQRYCNASVTNSVKGTNA

SEQ ID NO: 20 - protein, 384 aa, Artificial Sequence
  Description of Artificial Sequence Synthetic polypeptide
MRVLLAALGLLFLGALRAFPQDRPFEDTCHGNPSHYYDKAVRRCCYRCPMGLFPTQQPQRPTDCRKQCEPDYYLDEADRCTACVTCSRDDLVEKTPCAWNSSRVCECRPGMFCSTSAVNSCARCFFHSVCPAGMIVKFPGTAQKNTVCEPASPGVSPACASPENCKEPSSGTIPQAKPTPVSPATSSASTMPVRGGTRLAQEAASKLTRAPDSPSSVGRPSSDPGLSPTQPCPEGSGDCRKQCEPDYYLDEAGRCTACVSCSRDDLVEKTPCAWNSSRTCECRPGMICATSATNSCARCVPYPICAAETVTKPQDMAEKDTTFEAPPLGTQPDCNPTPENGEAPASTSPTQSLLVDSQASKTLPIPTSAPVALSSTGKPVLDAG

SEQ ID NO: 21 - protein, 144 aa, Artificial Sequence
  Description of Artificial Sequence Synthetic polypeptide
LACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLT

SEQ ID NO: 22 - protein, 205 aa, Artificial Sequence
  Description of Artificial Sequence Synthetic polypeptide
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDK

SEQ ID NO: 23 - protein, 6 aa, Artificial Sequence
  Description of Artificial Sequence Synthetic peptide
HPPNER

SEQ ID NO: 24 - protein, 33 aa, Artificial Sequence
  Description of Artificial Sequence Synthetic polypeptide
PTIASQPLSLRPEACRPAAGGAVHTRGLDFACD

SEQ ID NO: 25 - protein, 21 aa, Artificial Sequence
  Description of Artificial Sequence Synthetic peptide
THLVILGAILLCLGVALTFIF

SEQ ID NO: 26 - protein, 21 aa, Artificial Sequence
  Description of Artificial Sequence Synthetic peptide
IYIWAPLAGTCGVLLLSLVIT

SEQ ID NO: 27 - protein, 24 aa, Artificial Sequence
  Description of Artificial Sequence Synthetic peptide
PVLFWVILVLVVVVGSSAFLLCHR

SEQ ID NO: 28 - protein, 25 aa, Artificial Sequence
  Description of Artificial Sequence Synthetic peptide
SIISAVVGILLVVVLGVVFGILIKR

SEQ ID NO: 29 - protein, 3 aa, Artificial Sequence
  Description of Artificial Sequence Synthetic peptide
RLR

SEQ ID NO: 30 - protein, 6 aa, Artificial Sequence
  Description of Artificial Sequence Synthetic peptide
LYCWVR

SEQ ID NO: 31 - protein, 10 aa, Artificial Sequence
  Description of Artificial Sequence Synthetic peptide
TSYDYCRVPG

SEQ ID NO: 32 - protein, 9 aa, Artificial Sequence
  Description of Artificial Sequence Synthetic peptide
SDKQTLLPG

SEQ ID NO: 33 - protein, 106 aa, Artificial Sequence
  Description of Artificial Sequence Synthetic polypeptide
KINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 34 - protein, 35 aa, Artificial Sequence
  Description of Artificial Sequence Synthetic polypeptide
MDAMKRGLCCVLLLCGAVFVSPSQEIHARFRRGAR

SEQ ID NO: 35 - protein, 21 aa, Artificial Sequence
  Description of Artificial Sequence Synthetic peptide
MALPVTALLLPLALLLHAARP

SEQ ID NO: 36 - protein, 19 aa, Artificial Sequence
  Description of Artificial Sequence Synthetic peptide
MLLLVTSLLLCELPHPAFL

SEQ ID NO: 37 - protein, 29 aa, Artificial Sequence
  Description of Artificial Sequence Synthetic peptide
SQPLSLRPEACRPAAGGAVHTRGLDFACD

SEQ ID NO: 38 - protein, 110 aa, Artificial Sequence
  Description of Artificial Sequence Synthetic polypeptide
MLQMAGQCSQNEYFDSLLHACIPCQLRCSSNTPPLTCQRYCNASVTNSVKGTNAHPPNERTHLVILGAILLCLGVALTFIFRASTNRRKSGKYKKVEIKELGELRKEPSL

SEQ ID NO: 39 - DNA, 333 bp, Artificial Sequence
  Description of Artificial Sequence Synthetic polynucleotide
atgttgcaaatggctggtcaatgttcacagaatgaatacttcgatagtctgttgcacgcttgcattccatgtcaactgaggtgctctagcaatacacctccactcacttgccagagatactgcaacgcgtccgtaacaaactcagtcaagggcacgaatgcgcacccccccaatgaaagaactcacctggtcattctcggggctatcctcttgtgtctcggagtagcgttgacctttatcttccgcgcgtccacgaatcggaggaagagcggaaagtacaagaaggtggagattaaagaattgggggaactgcgcaaggagccaagtctgtaa

SEQ ID NO: 40 - protein, 28 aa, Artificial Sequence
  Description of Artificial Sequence Synthetic peptide
ASTNRRKSGKYKKVEIKELGELRKEPSL

SEQ ID NO: 41 - protein, 54 aa, Artificial Sequence; identical to SEQ ID NO: 19
  Description of Artificial Sequence Synthetic polypeptide

SEQ ID NO: 42 - DNA, 162 bp, Artificial Sequence
  Description of Artificial Sequence Synthetic polynucleotide
atgctccagatggctggccagtgttcgcagaacgagtacttcgacagtcttctacacgcctgtattccttgtcagctgcgttgctctagcaacacgcccccactgacctgccagcgttactgcaatgcttccgtcaccaactccgtgaagggcaccaacgcg

SEQ ID NO: 43 - protein, 706 aa, Homo sapiens
MASPADSCIQFTRHASDVLLNLNRLRSRDILTDVVIVVSREQFRAHKTVLMACSGLFYSIFTDQLKCNLSVINLDPEINPEGFCILLDFMYTSRLNLREGNIMAVMATAMYLQMEHVVDTCRKFIKASEAEMVSAIKPPREEFLNSRMLMPQDIMAYRGREVVENNLPLRSAPGCESRAFAPSLYSGLSTPPASYSMYSHLPVSSLLFSDEEFRDVRMPVANPFPKERALPCDSARPVPGEYSRPTLEVSPNVCHSNIYSPKETIPEEARSDMHYSVAEGLKPAAPSARNAPYFPCDKASKEEERPSSEDEIALHFEPPNAPLNRKGLVSPQSPQKSDCQPNSPTESCSSKNACILQASGSPPAKSPTDPKACNWKKYKFIVLNSLNQNAKPEGPEQAELGRLSPRAYTAPPACQPPMEPENLDLQSPTKLSASGEDSTIPQASRLNNIVNRSMTGSPRSSSESHSPLYMHPPKCTSCGSQSPQHAEMCLHTAGPTFPEEMGETQSEYSDSSCENGAFFCNECDCRFSEEASLKRHTLQTHSDKPYKCDRCQASFRYKGNLASHKTVHTGEKPYRCNICGAQFNRPANLKTHTRIHSGEKPYKCETCGARFVQVAHLRAHVLIHTGEKPYPCEICGTRFRHLQTLKSHLRIHTGEKPYHCEKCNLHFRHKSQLRLHLRQKHGAITNTKVQYRVSATDLPPELPKAC

SEQ ID NO: 44 - DNA, 2118 bp, Homo sapiens
atggcctcgccggctgacagctgtatccagttcacccgccatgccagtgatgttcttctcaaccttaatcgtctccggagtcgagacatcttgactgatgttgtcattgttgtgagccgtgagcagtttagagcccataaaacggtcctcatggcctgcagtggcctgttctatagcatctttacagaccagttgaaatgcaaccttagtgtgatcaatctagatcctgagatcaaccctgagggattctgcatcctcctggacttcatgtacacatctcggctcaatttgcgggagggcaacatcatggctgtgatggccacggctatgtacctgcagatggagcatgttgtggacacttgccggaagtttattaaggccagtgaagcagagatggtttctgccatcaagcctcctcgtgaagagttcctcaacagccggatgctgatgccccaagacatcatggcctatcggggtcgtgaggtggtggagaacaacctgccactgaggagcgcccctgggtgtgagagcagagcctttgcccccagcctgtacagtggcctgtccacaccgccagcctcttattccatgtacagccacctccctgtcagcagcctcctcttctccgatgaggagtttcgggatgtccggatgcctgtggccaaccccttccccaaggagcgggcactcccatgtgatagtgccaggccagtccctggtgagtacagccggccgactttggaggtgtcccccaatgtgtgccacagcaatatctattcacccaaggaaacaatcccagaagaggcacgaagtgatatgcactacagtgtggctgagggcctcaaacctgctgccccctcagcccgaaatgccccctacttcccttgtgacaaggccagcaaagaagaagagagaccctcctcggaagatgagattgccctgcatttcgagccccccaatgcacccctgaaccggaagggtctggttagtccacagagcccccagaaatctgactgccagcccaactcgcccacagagtcctgcagcagtaagaatgcctgcatcctccaggcttctggctcccctccagccaagagccccactgaccccaaagcctgcaactggaagaaatacaagttcatcgtgctcaacagcctcaaccagaatgccaaaccagaggggcctgagcaggctgagctgggccgcctttccccacgagcctacacggccccacctgcctgccagccacccatggagcctgagaaccttgacctccagtccccaaccaagctgagtgccagcggggaggactccaccatcccacaagccagccggctcaataacatcgttaacaggtccatgacgggctctccccgcagcagcagcgagagccactcaccactctacatgcaccccccgaagtgcacgtcctgcggctctcagtccccacagcatgcagagatgtgcctccacaccgctggccccacgttccctgaggagatgggagagacccagtctgagtactcagattctagctgtgagaacggggccttcttctgcaatgagtgtgactgccgcttctctgaggaggcctcactcaagaggcacacgctgcagacccacagtgacaaaccctacaagtgtgaccgctgccaggcctccttccgctacaagggcaacctcgccagccacaagaccgtccataccggtgagaaaccctatcgttgcaacatctgtggggcccagttcaaccggccagccaacctgaaaacccacactcgaattcactctggagagaagccctacaaatgcgaaacctgcggagccagatttgtacaggtggcccacctccgtgcccatgtgcttatccacactggtgagaagccctatccctgtgaaatctgtggcacccgtttccggcaccttcagactctgaagagccacctgcgaatccacacaggagagaaaccttaccattgtgagaagtgtaacctgcatttccgtcacaaaagccagctgcgacttcacttgcgccagaagcatggcgccatcaccaacaccaaggtgcaataccgcgtgtcagccactgacctgcctccggagctccccaaagcctgc

SEQ ID NO: 45 - protein, 706 aa, Artificial Sequence
  Description of Artificial Sequence Synthetic polypeptide
MASPADSCIQFTRHASDVLLNLNRLRSRDILTDVVIVVSREQFRAHKTVLMACSGLFYSIFTDQLKCNLSVINLDPEINPEGFCILLDFMYTSRLNLREGNIMAVMATAMYLQMEHVVDTCRKFIKASEAEMVSAIKPPREEFLNSRMLMPQDIMAYRGREVVENNLPLRSAPGCESRAFAPSLYSGLSTPPASYSMYSHLPVSSLLFSDEEFRDVRMPVANPFPKERALPCDSARPVPGEYSRPTLEVSPNVCHSNIYSPKETIPEEARSDMHYSVAEGLKPAAPSARNAPYFPCDKASKEEERPSSEDEIALHFEPPNAPLNRKGLVSPQSPQKSDCQPNSPTESCSSKNACILQASGSPPAKSPTDPKACNWKKYKFIVLNSLNQNAKPEGLEQAELGRLSPRAYTAPPACQPPMEPENLDLQSPTKLSASGEDSTIPQASRLNNIVNRSMTGSPRSSSESHSPLYMHPPKCTSCGSQSPQHAEMCLHTAGPTFPEEMGETQSEYSDSSCENGAFFCNECDCRFSEEASLKRHTLQTHSDKPYKCDRCQASFRYKGNLASHKTVHTGEKPYRCNICGAQFNRPANLKTHTRIHSGEKPYKCETCGARFVQVAHLRAHVLIHTGEKPYPCEICGTRFRHLQTLKSHLRIHTGEKPYHCEKCNLHFRHKSQLRLHLRQKHGAITNTKVQYRVSATDLPPELPKAC

SEQ ID NO: 46 - DNA, 2118 bp, Artificial Sequence
  Description of Artificial Sequence Synthetic polynucleotide
atggcctcgccggctgacagctgtatccagttcacccgccatgccagtgatgttcttctcaaccttaatcgtctccggagtcgagacatcttgactgatgttgtcattgttgtgagccgtgagcagtttagagcccataaaacggtcctcatggcctgcagtggcctgttctatagcatctttacagaccagttgaaatgcaaccttagtgtgatcaatctagatcctgagatcaaccctgagggattctgcatcctcctggacttcatgtacacatctcggctcaatttgcgggagggcaacatcatggctgtgatggccacggctatgtacctgcagatggagcatgttgtggacacttgccggaagtttattaaggccagtgaagcagagatggtttctgccatcaagcctcctcgtgaagagttcctcaacagccggatgctgatgccccaagacatcatggcctatcggggtcgtgaggtggtggagaacaacctgccactgaggagcgcccctgggtgtgagagcagagcctttgcccccagcctgtacagtggcctgtccacaccgccagcctcttattccatgtacagccacctccctgtcagcagcctcctcttctccgatgaggagtttcgggatgtccggatgcctgtggccaaccccttccccaaggagcgggcactcccatgtgatagtgccaggccagtccctggtgagtacagccggccgactttggaggtgtcccccaatgtgtgccacagcaatatctattcacccaaggaaacaatcccagaagaggcacgaagtgatatgcactacagtgtggctgagggcctcaaacctgctgccccctcagcccgaaatgccccctacttcccttgtgacaaggccagcaaagaagaagagagaccctcctcggaagatgagattgccctgcatttcgagccccccaatgcacccctgaaccggaagggtctggttagtccacagagcccccagaaatctgactgccagcccaactcgcccacagagtcctgcagcagtaagaatgcctgcatcctccaggcttctggctcccctccagccaagagccccactgaccccaaagcctgcaactggaagaaatacaagttcatcgtgctcaacagcctcaaccagaatgccaaaccagaggggcttgagcaggctgagctgggccgcctttccccacgagcctacacggccccacctgcctgccagccacccatggagcctgagaaccttgacctccagtccccaaccaagctgagtgccagcggggaggactccaccatcccacaagccagccggctcaataacatcgttaacaggtccatgacgggctctccccgcagcagcagcgagagccactcaccactctacatgcaccccccgaagtgcacgtcctgcggctctcagtccccacagcatgcagagatgtgcctccacaccgctggccccacgttccctgaggagatgggagagacccagtctgagtactcagattctagctgtgagaacggggccttcttctgcaatgagtgtgactgccgcttctctgaggaggcctcactcaagaggcacacgctgcagacccacagtgacaaaccctacaagtgtgaccgctgccaggcctccttccgctacaagggcaacctcgccagccacaagaccgtccataccggtgagaaaccctatcgttgcaacatctgtggggcccagttcaaccggccagccaacctgaaaacccacactcgaattcactctggagagaagccctacaaatgcgaaacctgcggagccagatttgtacaggtggcccacctccgtgcccatgtgcttatccacactggtgagaagccctatccctgtgaaatctgtggcacccgtttccggcaccttcagactctgaagagccacctgcgaatccacacaggagagaaaccttaccattgtgagaagtgtaacctgcatttccgtcacaaaagccagctgcgacttcacttgcgccagaagcatggcgccatcaccaacaccaaggtgcaataccgcgtgtcagccactgacctgcctccggagctccccaaagcctgc

SEQ ID NO: 47 - protein, 515 aa, Artificial Sequence
  Description of Artificial Sequence Synthetic polypeptide
MRVLLAALGLLFLGALRAFPQDRPFEDTCHGNPSHYYDKAVRRCCYRCPMGLFPTQQCPQRPTDCRKQCEPDYYLDEADRCTACVTCSRDDLVEKTPCAWNSSRVCECRPGMFCSTSAVNSCARCFFHSVCPAGMIVKFPGTAQKNTVCEPASPGVSPACASPENCKEPSSGTIPQAKPTPVSPATSSASTMPVRGGTRLAQEAASKLTRAPDSPSSVGRPSSDPGLSPTQPCPEGSGDCRKQCEPDYYLDEAGRCTACVSCSRDDLVEKTPCAWNSSRTCECRPGMICATSATNSCARCVPYPICAAETVTKPQDMAEKDTTFEAPPLGTQPDCNPTPENGEAPASTSPTQSLLVDSQASKTLPIPTSAPVALSSTGKPVLDAGPVLFWVILVLVVVVGSSAFLLCHRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 48 - DNA, 1545 bp, Artificial Sequence; identical to SEQ ID NO: 10
  Description of Artificial Sequence Synthetic polynucleotide

SEQ ID NO: 49 - protein, 595 aa, Homo sapiens
MRVLLAALGLLFLGALRAFPQDRPFEDTCHGNPSHYYDKAVRRCCYRCPMGLFPTQQCPQRPTDCRKQCEPDYYLDEADRCTACVTCSRDDLVEKTPCAWNSSRVCECRPGMFCSTSAVNSCARCFFHSVCPAGMIVKFPGTAQKNTVCEPASPGVSPACASPENCKEPSSGTIPQAKPTPVSPATSSASTMPVRGGTRLAQEAASKLTRAPDSPSSVGRPSSDPGLSPTQPCPEGSGDCRKQCEPDYYLDEAGRCTACVSCSRDDLVEKTPCAWNSSRTCECRPGMICATSATNSCARCVPYPICAAETVTKPQDMAEKDTTFEAPPLGTQPDCNPTPENGEAPASTSPTQSLLVDSQASKTLPIPTSAPVALSSTGKPVLDAGPVLFWVILVLVVVVGSSAFLLCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 50 - DNA, 1788 bp, Homo sapiens
atgcgcgtcctcctcgccgcgctgggactgctgttcctgggggcgctacgagccttcccacaggatcgacccttcgaggacacctgtcatggaaaccccagccactactatgacaaggctgtcaggaggtgctgttaccgctgccccatggggctgttcccgacacagcagtgcccacagaggcctactgactgcaggaagcagtgtgagcctgactactacctggatgaggccgaccgctgtacagcctgcgtgacttgttctcgagacgacctcgtggagaagacgccgtgtgcatggaactcctcccgtgtctgcgaatgtcgacccggcatgttctgttccacgtctgccgtcaactcctgtgcccgctgcttcttccattctgtctgtccggcagggatgattgtcaagttcccaggcacggcgcagaagaacacggtctgtgagccggcttccccaggggtcagccctgcctgtgccagcccagagaactgcaaggaaccctccagtggcaccatcccccaggccaagcccaccccggtgtccccagcaacctccagtgccagcaccatgcctgtaagagggggcacccgcctcgcccaggaagctgcttctaaactgacgagggctcccgactctccctcctctgtgggaaggcctagttcagatccaggtctgtccccaacacagccatgcccagaggggtctggtgattgcagaaagcagtgtgagcccgactactacctggacgaggccggccgctgcacggcctgcgtgagctgttctcgagatgaccttgtggagaagacgccatgtgcatggaactcctcccgcacctgcgaatgtcgacctggcatgatctgtgccacatcagccaccaactcctgtgcccgctgtgtcccctacccaatctgtgcagcagagacggtcaccaagccccaggatatggctgagaaggacaccacctttgaggcgccacccctggggacccagccggactgcaaccccaccccagagaatggcgaggcgcctgccagcaccagccccactcagagcttgctggtggactcccaggccagtaagacgctgcccatcccaaccagcgctcccgtcgctctctcctccacggggaagcccgttctggatgcagggccagtgctcttctgggtgatcctggtgttggttgtggtggtcggctccagcgccttcctcctgtgccaccggagggcctgcaggaagcgaattcggcagaagctccacctgtgctacccggtccagacctcccagcccaagctagagcttgtggattccagacccaggaggagctcaacgcagctgaggagtggtgcgtcggtgacagaacccgtcgcggaagagcgagggttaatgagccagccactgatggagacctgccacagcgtgggggcagcctacctggagagcctgccgctgcaggatgccagcccggccgggggcccctcgtcccccagggaccttcctgagccccgggtgtccacggagcacaccaataacaagattgagaaaatctacatcatgaaggctgacaccgtgatcgtggggaccgtgaaggctgagctgccggagggccggggcctggcggggccagcagagcccgagttggaggaggagctggaggcggaccataccccccactaccccgagcaggagacagaaccgcctctgggcagctgcagcgatgtcatgctctcagtggaagaggaagggaaagaagaccccttgcccacagctgcctctggaaagtga

SEQ ID NO: 51 - protein, 21 aa, Artificial Sequence
  Description of Artificial Sequence Synthetic peptide
PVLFWVILVLVVVVGSSAFLL